(19) United States
(12) Patent Application Publication
(10) Pub. No.: US 2013/0029358 A1
(43) Pub. Date: Jan. 31, 2013

(54) IMMUNODOMINANT MHC DR52B RESTRICTED NY-ESO-1 EPITOPES, MHC CLASS II MONOMERS AND MULTIMERS, AND USES THEREOF

(71) Applicants: Danila Valmori, Saint Herblain (FR); Maha Ayyoub, Saint Herblain (FR); Immanuel F. Luescher, Gollion (CH); Danijel Dojcinovic, Epalinges (CH)

(72) Inventors: Danila Valmori, Saint Herblain (FR); Maha Ayyoub, Saint Herblain (FR); Immanuel F. Luescher, Gollion (CH); Danijel Dojcinovic, Epalinges (CH)

(73) Assignee: LUDWIG INSTITUTE FOR CANCER RESEARCH LTD., New York, NY (US)

(21) Appl. No.: 13/499,771

(22) PCT Filed: Oct. 1, 2010
(86) PCT No.: PCT/US10/02668

§ 371 Date: Oct. 12, 2012

Related U.S. Application Data
(60) Provisional application No. 61/320,060, filed on Apr. 1, 2010.

(51) Int. Cl. C07K 7/06; C07K 7/08; G01N 21/64; C07K 2/00; C07K 14/74; G01N 33/566; C07K 14/47

(52) U.S. Cl. 435/7.24; 530/328; 530/326; 530/327; 530/325; 530/324; 530/300; 530/409; 530/350; 530/395

(57) ABSTRACT

Immunostimulatory NY-ESO-1 epitopes recognized by MHC-DRB3*0202 (DR52b) or DRB1*0101 (DR1) restricted T cells are described. Methods for their use in diagnostic and therapeutic approaches are also provided. Further, methods for the generation and isolation of MHC class II molecules, either “empty” or peptide-loaded, are provided. Methods for the assembly of MHC class II multimers, for example, tetramers, are also provided. Methods for the detection of T cells binding to specific peptide-loaded MHC class II molecules are also described herein.

### RELATED APPLICATIONS

This application claims the benefit 35 U.S.C. §119(e) of U.S. Provisional Patent Application Ser. No. 61/278,051, filed Oct. 2, 2009, and U.S. Provisional Patent Application Ser. No. 61/320,060, filed Apr. 1, 2010. The entire disclosures of both Applications are incorporated herein by reference.

### BACKGROUND OF THE INVENTION

Tumor antigens, for example NY-ESO-1, are able to elicit immune responses in the autologous host. Accordingly, such tumor antigens are potentially useful for tumor therapy. Some therapeutic approaches employing such an anti-tumor immune responses include the identification of immunostimulatory tumor antigen epitopes. Once identified, a tumor antigen can be administered to a subject having a tumor expressing the antigen to elicit an immune response that specifically targets tumor cells. Translation of this therapeutic paradigm into clinical applications is hampered, however, because (i) only few epitopes of tumor antigens have been identified, and (ii) reliable methods and reagents for monitoring immune responses to tumor antigens are lacking.

### SUMMARY OF THE INVENTION

Active elicitation or enhancement of a tumor-specific immune response through vaccination of a tumor-bearing subject is an attractive therapeutic paradigm that could be used in the therapy of hyperproliferative disease either alone or in combination with conventional therapeutic methods. Such an immune-response based therapeutic approach, ideally in combination with immunomodulation, is presently viewed as a strategy that could potentially treat, prevent disease recurrence or/and lead to stabilization of hyperproliferative disease, improving the long-term outcome of current treatment of such diseases, for example, of cancer. Such a vaccination approach generally involves the administration to a subject having a hyperproliferative disease an immunostimulatory epitope of a tumor antigen that is specifically expressed by neoplastic cells, e.g., cells of a tumor, in order to elicit or enhance an immune response specifically targeting the antigen-expressing neoplastic cells, but not directed towards non-expressing, healthy cells.

Sensitive methods to monitor the induction or enhancement of an immune response in a subject, for example in response to a tumor or to the administration of an immunostimulatory epitope of a tumor antigen, would be beneficial for tumor diagnostics and classification as well as for the development of therapeutic approaches involving the immune system of a subject carrying or suspected to carry a tumor. Such methods generally involve the analysis of T-cell populations mediating the immune response, for example, CD8+ T-cells, if the immunogenic epitope is presented by MHC class I molecules, and/or CD4+ T-cells, if the immunogenic epitope is presented by MHC class II molecules.

Sensitive detection and analysis of antigen-specific T-cell populations is typically carried out by staining a T-cell population with multimers of MHC molecules (either class I or class II) that are loaded with the antigenic peptide in question. Importantly, MHC class I and class II molecules differ significantly in their structure, the type of antigenic peptide that can be bound, and the stability of empty and peptide-loaded MHC molecule complexes. MHC class I molecules comprise one type α heavy chain that is divided into three domains (α1, α2, and α3) and associated with a (32 microglobulin molecule. The α1 and α2 domains fold to make up a closed-end groove that typically binds an antigenic peptide of 8-10 amino acid residues in length. In contrast, MHC class II molecules comprise one type α heavy chain and one type β heavy chain, both of which are divided into two domains (α1, and α2; and β1 and β2, respectively). The antigen-binding groove of MHC class II molecules is open at both ends and the antigens presented by MHC class II molecules are, accordingly, longer, typically between about 15 and about 24 amino acid residues in length. Based on their different structure, MHC class I and MHC class II molecules each present unique technical challenges in regard to the production of antigenic peptide-loaded MHC multimers and detection and analysis of antigen-specific T cells by MHC class II-peptide multimers (e.g., tetramers) lags behind MHC class I systems, which to a large extent is due to inadequate reagent quality.

Some aspects of this invention provide universally applicable methods for the preparation of isolated MHC II-peptide staining reagents. For example, some aspects of this invention provide methods for the isolation of MHC class II molecules that have stably bound a peptide of interest by using a tag conjugated to the peptide.

In contrast to recombinant peptide-loaded MHC class I molecules, which can be obtained by peptide driven refolding, recombinant MHC class II proteins typically require significant genetic engineering and more cost- and time-intensive methods of production. In most cases “empty” (without nominal peptide cargo) MHC class II molecules are isolated from insect cell culture supernatants and subsequently loaded with a peptide of interest. Peptide-loading is often inefficient, particularly for peptides of low binding strength, and the resulting complexes are of limited stability. Further, the staining of CD4+ T cells with MHC class II multimers is often weak, frequently rendering detection of low frequency CD4+ T cells inconclusive. Based on the significant differences in binding avidity of MHC class I and class II multimers to their target T cells, staining methods for both classes differ substantially, for example, in both time of exposure to staining multimers and in optimum temperature for staining.

Some aspects of this invention provide agents and methods for the generation of peptide-loaded MHC class II molecules. In some embodiments, molecularly defined monomers are produced, for example, MHC class II monomers that are loaded with the peptide of interest. In some embodiments, the peptide of interest is conjugated to a tag. In some embodiment, the tag is a peptide tag, for example, a peptide tag that is N-terminally or C-terminally fused to the antigenic peptide of interest, allowing for the isolation of correctly loaded MHC class II molecules by affinity chromatography. In some embodiments, the peptide tag is a polyhistidine tag. Accordingly, some aspects of this invention provide methods for the generation of peptide-loaded MHC class II molecules that include purification of MHC class II molecules loaded with a peptide conjugated to a tag by affinity chromatography, preferably under non-denaturing conditions. Some aspects of this invention provide methods for the generation of MHC class II multimers. In some embodiments, MHC class II multimers are generated from isolated, peptide-loaded MHC class II molecules, for example, peptide-loaded MHC class II molecules generated by methods described herein. In some embodiments, peptide-loaded MHC class II molecules are assembled into multimers by reaction with a multivalent binding molecule, for example, streptavidin.

Some aspects of this invention relate to improved methods for detection of CD4+ T cells by staining them with MHC class II multimers. In some embodiments, cells are contacted with an MHC class II multimer as described herein. Some aspects of this invention relate to the surprising discovery that desialylation of cells can improve MHC class II multimer staining several fold. Accordingly, improved MHC multimer staining methods including a desialylation step, for example, a step of enzymatic desialylation of the target cells.

Another important aspect in immune-response based therapeutic approaches is the identification of suitable tumor antigens and the determination of immunodominant epitopes within the amino acid sequences of such antigens. Among human tumor antigens (Cancer Immunity Peptide Database), cancer/testis antigens (CTA) are characterized by their expression pattern being restricted, in adult tissues, to testis and tumor tissues (1*). NY-ESO-1 (also referred to herein as ESO), a member of the CTA group, is expressed in a variety of tumors of different histological origin and displays significant spontaneous immunogenicity in patients bearing antigen-expressing tumors (2*). Candidate anti-cancer vaccines using various ESO-based immunogens are currently under trial (Cancer Vaccine Collaborative: www.cancerresearch.org).

Several studies have analyzed ESO-specific T cell responses in patients with natural anti-ESO immunity and reported the identification of regions of the ESO protein frequently recognized by T cells from different patients. For CD4⁺ T cells, two main immunodominant regions have been identified, ESO₈₁₋₁₀₀, recognized by CD4⁺ T cells from about half of the patients with spontaneous immunity to ESO, and ESO₁₁₉₋₁₄₃, recognized by CD4⁺ T cells from the large majority of the patients (3-7*). CD4⁺ T cell responses to a third region, ESO₁₅₇₋₁₇₀, frequently recognized by ESO-specific CTL (2), were also initially reported to be immunodominant but have not been frequently reported in subsequent studies (8*).

In a recent vaccination trial using as immunogen rESO administered with Montanide ISA-51 and CpG ODN 7909 to patients with no detectable pre-existing immunity to ESO, we obtained induction of significant T_{H}1 ESO-specific CD4⁺ T cell responses in all patients (9*). Analysis of the fine specificity of vaccine induced CD4⁺ T cells revealed that in all patients a sizable fraction of these responses were directed against ESO₁₁₉₋₁₄₃. Starting from the analysis of one patient with the highest ex-vivo detectable CD8⁺ and CD4⁺ T cell responses, we demonstrate in this study that vaccination with the full-length recombinant protein induces ESO₁₁₉₋₁₄₃-specific CD4⁺ T cells restricted by HLA-DR52b (DRB3*0202), an allele that is expressed by about half of the Caucasian population. Furthermore, we show that such responses can be detected in all vaccinated patients expressing DR52b, demonstrating the immunodominant nature of DR52b-restricted ESO₁₁₉₋₁₄₃-specific CD4⁺ T cell responses after vaccination with rESO. Finally, we found significant conservation of TCR usage for ESO-specific DR52b-restricted CD4⁺ T cells from different individuals. *References 1-9 of this paragraph and the two paragraphs immediately above are given in Example 1.

Some aspects of this invention provide isolated immunogenic or immunostimulatory, NY-ESO-1 peptides. Some aspects of this invention provide MHC class II molecules bound by an immunogenic or immunostimulatory NY-ESO-1 peptide. Some aspects of this invention provide MHC class II molecules bound to a tagged peptide. Some aspects of this invention provide multimers of NY-ESO-1-loaded MHC class II molecules. Some aspects of this invention provide multimers of MHC class II molecules loaded with a tagged peptide. Some aspects of this invention provide methods for eliciting an immune response in a subject by administering an immunogenic or immunostimulatory NY-ESO-1 peptide to a subject. Some aspects of this invention provide methods to induce or enhance proliferation of cells, for example, specific T-cells, by contacting them with an immunogenic or immunostimulatory NY-ESO-1 peptide. Some aspects of this invention provide methods to detect cells binding NY-ESO-1 peptide-loaded MHC class II molecules, for example, specific T-cells, by contacting them with an NY-ESO-1 peptide-loaded MHC class II molecule or multimer and detecting the bound molecules or multimers. Some aspects of this invention provide methods for measuring an immune response using MHC class II multimers, for example, tetramers. Some aspects of this invention provide methods for the generation of MHC class II molecules loaded with tagged peptides. Some aspects of this invention provide methods for the generation of MHC class II multimers, for example, tetramers, comprising MHC class II molecules loaded with tagged peptides. Some aspects of this invention provide methods for determining or identifying an epitope of a protein antigen using MHC class II molecules or multimers. Some aspects of the invention provide kits including an isolated NY-ESO-1 peptide, and/or MHC class II molecules in multimeric form.

In some embodiments, an isolated NY-ESO-1 peptide-loaded MHC class II molecule is provided, comprising a beta chain encoded by a DRB1 allele or a DRB3 allele, and an NY-ESO-1 peptide, the peptide comprising 9-25 contiguous amino acids of NY-ESO-1 (SEQ ID NO: 1), and/or an amino acid sequence selected from the group consisting of peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1. In some embodiments, the MHC class II protein comprises a DR52b beta chain and/or is encoded by a DRB3*0202 allele. In some embodiments, the MHC class II protein comprises a DR1 beta chain and/or is encoded by a DRB1*0101 allele. In some embodiments, the NY-ESO-1 peptide comprises the sequence TVSGNILTI (SEQ ID NO: 59). In some embodiments, the NY-ESO-1 peptide comprises the sequence EFTVSGNILTI (SEQ ID NO: 60). In some embodiments, the MHC class II molecule is linked to a ligand of a multivalent binding molecule. In some embodiments, the MHC class II molecule is linked to the ligand by covalent linkage. In some embodiments, the covalent linkage is a peptide bond, such that the MHC class II molecule and the ligand are linked as a fusion protein. In some embodiments, the ligand binds to a multivalent binding molecule. In some embodiments, the multivalent binding molecule binds at least one additional MHC class II molecule, wherein each additional MHC class II molecule is optionally peptide-loaded. In some embodiments, the multivalent binding molecule binds three additional MHC class II molecules, each one optionally peptide-loaded. In some embodiments, the ligand is biotin and the multivalent binding molecule is streptavidin or avidin. In some embodiments, the MHC class II molecule is a HLA class II molecule. In some embodiments, the NY-ESO-1 peptide is fused to a tag. In some embodiments, the tag is a poly-Histidine tag. In some embodiments, the tag comprises between 3 and 12 contiguous Histidine residues. In some embodiments, the NY-ESO-1 peptide is a fragment of an NY-ESO-1 protein.

In some embodiments, an isolated peptide-loaded MHC class II molecule is provided, comprising an MHC class II alpha chain, an MHC class II beta chain, and a tagged MHC-class II binding peptide. In some embodiments, the MHC class II protein comprises a DR52b beta chain and/or is encoded by a DRB3*0202 allele. In some embodiments, the MHC class II protein comprises a DR1 beta chain and/or is encoded by a DRB1*0101 allele. In some embodiments, the tagged peptide is a tagged NY-ESO-1 peptide comprising an amino acid sequence chosen from the sequences provided in SEQ ID NOs 2-60, and/or comprising 9-25 contiguous amino acids of NY-ESO-1 (SEQ ID NO: 1), and/or an amino acid sequence selected from the group consisting of peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1. In some embodiments, the MHC class II alpha chain and/or the MHC class II beta chain is a DM chain, a DO chain, a DP chain, a DQ chain, a DQ chain, or a DR chain. In some embodiments, the MHC class II molecule is linked to a ligand of a multivalent binding molecule. In some embodiments, the MHC class II molecule is linked to the ligand by covalent linkage. In some embodiments, the covalent linkage is a peptide bond, such that the MHC class II molecule and the ligand are linked as a fusion protein. In some embodiments, the ligand binds to a multivalent binding molecule. In some embodiments, the multivalent binding molecule binds at least one additional MHC class II molecule, wherein each additional MHC class II molecule is optionally peptide-loaded. In some embodiments, the multivalent binding molecule binds three additional MHC class II molecules, each one optionally peptide-loaded. In some embodiments, the ligand is biotin and the multivalent binding molecule is streptavidin or avidin. In some embodiments, the MHC class II molecule is a HLA class II molecule. In some embodiments, the tagged MHC class II binding peptide comprises a tag that is fused to the peptide. In some embodiments, the tag is a poly-Histidine tag. In some embodiments, the tag comprises between 3 and 12 contiguous Histidine residues. In some embodiments, the NY-ESO-1 peptide is a fragment of an NY-ESO-1 protein.

In some embodiments, an isolated MHC class II multimer is provided, comprising a multivalent binding molecule, an isolated NY-ESO-1 peptide-loaded MHC class II molecule, comprising an MHC class II molecule, comprising a beta chain encoded by a DR1 allele or a DRB3 allele, and bound to a NY-ESO-1 peptide, the NY-ESO-1 peptide comprising 9-25 contiguous amino acids of NY-ESO-1 (SEQ ID NO: 1), and/or an amino acid sequence selected from the group consisting of peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1, wherein the MHC class II molecule is linked to a ligand of the multivalent binding molecule, and at least one additional MHC class II molecule linked to a ligand of the multivalent binding molecule, wherein each of the at least one additional MHC class II molecule is optionally peptide-loaded, and wherein the ligands bind to the multivalent binding molecule. In some embodiments, an isolated MHC class II tetramer is provided, comprising a multivalent binding molecule, an isolated, NY-ESO-1 peptide-loaded MHC class II molecule, comprising a beta chain encoded by a DRB1 or a DRB3 allele, the NY-ESO-1 peptide comprising 9-25 contiguous amino acids of NY-ESO-1 (SEQ ID NO: 1), and/or an amino acid sequence selected from the group consisting of peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1, wherein the MHC class II molecule is linked to a ligand of the multivalent binding molecule, and three additional MHC class II molecule linked to a ligand of the multivalent binding molecule, wherein each of the three additional MHC class II molecule is optionally peptide-loaded, and wherein the ligands bind to the multivalent binding molecule. In some embodiments, the ligand is biotin and the multivalent binding molecule is streptavidin or avidin. In some embodiments, a MHC class II molecule of the multimer or the tetramer is loaded with a NY-ESO-1 peptide comprising an amino acid sequence starting at residue 123 and ending at residue 137 of SEQ ID NO: 1. In some embodiments, a MHC class II molecule of the multimer or the tetramer is loaded with a NY-ESO-1 peptide comprising the sequence TVSGNILTI (SEQ ID NO: 59). In some embodiments, a MHC class II molecule of the multimer or the tetramer is loaded with a NY-ESO-1 peptide comprising the sequence EFTVSGNILTI (SEQ ID NO: 60). In some embodiments, the multimer or tetramer is labeled with a detectable label. In some embodiments, the detectable label is a fluorophore suitable for fluorescence activated cell sorting (FACS). In some embodiments, the MHC class II multimer comprises at least one HLA class II molecule. In some embodiments, the NY-ESO-1 peptide is fused to a tag. In some embodiments, the tag is a poly-Histidine tag. In some embodiments, the tag comprises between 3 and 12 contiguous Histidine residues. In some embodiments, the NY-ESO-1 peptide is a fragment of an NY-ESO-1 protein.

In some embodiments, an isolated MHC class II multimer is provided, comprising a multivalent binding molecule, an isolated peptide-loaded MHC class II molecule, comprising an MHC class II alpha chain, an MHC class II beta chain, and a tagged MHC-class II binding peptide, wherein the MHC class II molecule is linked to a ligand of the multivalent binding molecule, and at least one additional MHC class II molecule linked to a ligand of the multivalent binding molecule, wherein each of the at least one additional MHC class II molecule is optionally peptide-loaded, and wherein the ligands of the isolated peptide-loaded MHC class II molecule and of the at least one additional MHC class II molecule bind to the multivalent binding molecule. In some embodiments, an isolated MHC class II tetramer is provided, comprising a multivalent binding molecule, an isolated, peptide-loaded MHC class II molecule, comprising an MHC class II alpha chain, an MHC class II beta chain, and a tagged MHC-class II binding peptide. wherein the MHC class II molecule is linked to a ligand of the multivalent binding molecule, and three additional MHC class II molecule linked to a ligand of the multivalent binding molecule, wherein each of the three additional MHC class II molecule is optionally peptide-loaded, and wherein the ligands of the isolated peptide-loaded MHC class II molecule and of the three additional MHC class II molecules bind to the multivalent binding molecule. In some embodiments, the ligand is biotin and the multivalent binding molecule is streptavidin or avidin. In some embodiments, the tagged MHC class II binding peptide comprises an amino acid sequence chosen from the sequences provided in SEQ ID NOs 2-60, and/or comprises 9-25 contiguous amino acids of NY-ESO-1 (SEQ ID NO: 1), and/or an amino acid sequence selected from the group consisting of peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1. In some embodiments, the multimer or tetramer is labeled with a detectable label. In some embodiments, the detectable label is a fluorophore suitable for fluorescence activated cell sorting (FACS). In some embodiments, the MHC class II multimer comprises at least one HLA class II molecule. In some embodiments, the tagged MHC class II binding peptide comprises a tag that is fused to the peptide. In some embodiments, the tag is a poly-Histidine tag. In some embodiments, the tag comprises between 3 and 12 contiguous Histidine residues.

In some embodiments, a method is provided, comprising administering to a HLA-DRB3*0202 (DR52b)-positive subject an immunostimulatory NY-ESO-1 peptide that is able to specifically bind an HLA-DRB3*0202 (DR52b) protein, the NY-ESO-1 peptide comprising 9-25 contiguous amino acids of NY-ESO-1 (SEQ ID NO: 1), and/or an amino acid sequence chosen from a list comprising peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1 in an amount sufficient to elicit an immune response. In some embodiments, a method is provided, comprising administering to a HLA-DRB1*0101 (DR1)-positive subject an immunostimulatory NY-ESO-1 peptide that is able to specifically bind an HLA-DRB1*0101 (DR1) protein, the NY-ESO-1 peptide comprising 9-25 contiguous amino acids of NY-ESO-1 (SEQ ID NO: 1), and/or an amino acid sequence chosen from a list comprising peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1 in an amount sufficient to elicit an immune response. In some embodiments, a method is provided, comprising determining the presence and/or expression of a HLA-DRB3*0202 allele and/or the presence of HLA-DRB3*0202 (DR52b) restricted T cells in a subject, and, if the subject carries and/or expresses a HLA-DRB3*0202 allele and/or carries HLA-DRB3*0202 (DR52b) restricted T cells, administering to the subject an immunostimulatory NY-ESO-1 peptide that is able to specifically bind an HLA-DR133*0202 (DR52b) protein, the NY-ESO-1 peptide comprising 9-25 contiguous amino acids of NY-ESO-1 (SEQ ID NO: 1), and/or an amino acid sequence chosen from a list comprising peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1 in an amount sufficient to elicit an immune response; or if the subject does not carry and/or express a HLA-DRB3*0202 allele and/or carries HLA-DRB3*0202 (DR52b) restricted T cells, not administering to the subject an immunostimulatory NY-ESO-1 peptide that is able to specifically bind an HLA-DRB3*0202 (DR52b) protein. In some embodiments, a method is provided, comprising determining the presence and/or expression of a HLA-DRB1*0101 allele and/or the presence of HLA-DRB1*0101 (DR1) restricted T cells in a subject, and, if the subject carries and/or expresses a HLA-DRB1*0101 allele and/or carries HLA-DRB1*0101 (DR1) restricted T cells, administering to the subject an immunostimulatory NY-ESO-1 peptide that is able to specifically bind an HLA-DRB1*0101 (DR1) protein, the NY-ESO-1 peptide comprising 9-25 contiguous amino acids of NY-ESO-1 (SEQ ID NO: 1), and/or an amino acid sequence chosen from a list comprising peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1 in an amount sufficient to elicit an immune response; or if the subject does not carry and/or express a HLA-DRB1*0101 allele and/or carries HLA-DRB1*0101 (DR1) restricted T cells, not administering to the subject an immunostimulatory NY-ESO-1 peptide that is able to specifically bind an HLA-DRB1*0101 (DR1) protein. In some embodiments, the NY-ESO-1 peptide comprises the sequence TVSGNILTI (SEQ ID NO: 59). In some embodiments, the NY-ESO-1 peptide comprises the sequence EFTVSGNILTI (SEQ ID NO: 60). In some embodiments, the NY-ESO-1 peptide is bound by a HLA-DRB3*0202 (DR52b) restricted CD4⁺ T cell or a DRB1*0101 (DR1) restricted CD4⁺ cell. In some embodiments, the subject is diagnosed to have a tumor, a cell of which expresses NY-ESO-1. In some embodiments, the immune response is induction of HLA-DRB3*0202 (HLA-DR52b) restricted CD4+ T cells and/or of HLA-DRB1*0101 (HLA-DR1) restricted CD4+ T cells specific for the NY-ESO-1 peptide. In some embodiments, the immune response is increasing the rate of proliferation of HLA-DRB3*0202 (DR52b) and/or of HLA-DRB1*0101 (HLA-DR1) restricted CD4+ T cells restricted CD4+ T cells specific for the NY-ESO-1 peptide. In some embodiments, the immunostimulatory NY-ESO-1 peptide is administered to the subject based on the subject carrying and/or expressing a HLA-DRB3*0202 allele and/or a HLA-DRB1*0101 allele and/or the presence of HLA-DRB3*0202 (DR52b) restricted T cells and/or the presence of HLA-DRB1*0101 (HLA-DR1) restricted CD4+ T cells in the subject. In some embodiments, the immunostimulatory NY-ESO-1 peptide is a fragment of a NY-ESO-1 protein.

In some embodiments, a method, comprising obtaining a cell population from a subject, contacting the cell population with an immunostimulatory NY-ESO-1 peptide and/or a cell expressing an immunostimulatory NY-ESO-1 peptide comprising at least 9 contiguous amino acid residues of SEQ ID NO: 1, wherein the peptide is able to bind an MHC class II molecule that comprises a beta chain encoded by a DRB3*0202 allele or a DRB1*0101 allele, thus inducing or increasing proliferation of a DRB3*0202 (DR52b) restricted CD4⁺ T cell and/or a DRB1*0101 (DR1) restricted CD4⁺ T cell, respectively, specifically recognizing the immunostimulatory NY-ESO-1 peptide, optionally isolating a DRB3*0202 (MHC-DR52b) restricted CD4⁺ T cell or a DRB1*0101 (MHC-DR1) restricted CD4⁺ T cell specifically recognizing the immunostimulatory NY-ESO-1 peptide from the cell population, and optionally administering the DRB3*0202 (DR52b) restricted CD4⁺ T cell or the a DRB1*0101 (MHC-DR1) restricted CD4⁺ T cell specifically recognizing the immunostimulatory NY-ESO-1 peptide to the subject. In some embodiments, the immunostimulatory NY-ESO-1 peptide comprises 9-25 contiguous amino acids of NY-ESO-1 (SEQ ID NO: 1). In some embodiments, the immunostimulatory NY-ESO-1 peptide comprises an amino acid sequence selected from the group consisting of peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1. In some embodiments, the immunostimulatory NY-ESO-1 peptide comprises an amino acid sequence starting at residue 123 and ending at residue 137 of SEQ ID NO: 1. In some embodiments, the immunostimulatory NY-ESO-1 peptide comprises the sequence TVSGNILTI (SEQ ID NO: 59). In some embodiments, the immunostimulatory NY-ESO-1 peptide comprises the sequence EFTVSGNILTI (SEQ ID NO: 60). In some embodiments, the subject is diagnosed to have a tumor, a cell of which expresses NY-ESO-1. In some embodiments, the immunostimulatory NY-ESO-1 peptide is a fragment of a NY-ESO-1 protein. In some embodiments, the isolating a DRB3*0202 (MHC-DR52b) restricted CD4⁺ T cell or a DRB1*0101 (MHC-DR1) restricted CD4⁺ T cell specifically recognizing the immunostimulatory NY-ESO-1 peptide from the cell population comprises contacting the cell population with a peptide-loaded MHC class II molecule, multimer or tetramer as described herein and detecting the peptide-loaded MHC class II molecule, multimer or tetramer on the surface of the contacted cells, wherein, if the molecule, multimer, or tetramer is detected on the surface of a T-cell, the T-cell is determined to be a DRB3*0202 (MHC-DR52b) restricted CD4⁺ T cell or a DRB1*0101 (MHC-DR1) restricted CD4⁺ T cell, respectively, specifically recognizing the immunostimulatory NY-ESO-1 peptide, and is isolated from the cell population. In some embodiments, the DRB3*0202 (MHC-DR52b) restricted CD4⁺ T cell or the a DRB1*0101 (MHC-DR1) restricted CD4⁺ T cell specifically recognizing the immunostimulatory NY-ESO-1 peptide is isolated from the cell population by FACS. In some embodiments, the cell population is a peripheral blood cell population, a thymus cell population, a cord blood cell population, a lymph node cell population, a tumor infiltrating lymphocyte population, and/or a normal or inflamed tissue infiltrating lymphocyte population. In some embodiments, the cell population is a T-cell population.

In some embodiments, a method is provided, comprising contacting a cell with a MHC class II multimer or tetramer under conditions suitable for binding of the tetramer or multimer to a T cell receptor, wherein at least one of the MHC class II molecules of the multimer or tetramer is loaded with a NY-ESO-1 peptide comprising at least 9 contiguous amino acids of SEQ ID NO: 1, and wherein the tetramer or multimer is, optionally, labeled with a detection agent, and detecting whether the cell binds the multimer or tetramer. In some embodiments, the peptide-loaded MHC class II molecule comprises a beta chain encoded by a DRB3 allele or a DRB1 allele. In some embodiments, the DRB3 allele is a DRB3*0202 allele and/or encodes a DR52b protein. In some embodiments, the DRB1 allele is a DRB1*0101 allele and/or encodes a DR1 protein. In some embodiments, the NY-ESO-1 peptide binds to a DRB3*0202 (DR52b) restricted T cell or to a DRB1*0101 (DR1) restricted T cell, the NY-ESO-1 peptide comprising 9-25 contiguous amino acids of NY-ESO-1 (SEQ ID NO: 1). In some embodiments, a MHC class II molecule of the multimer or the tetramer is loaded with a NY-ESO-1 peptide that binds to a DRB3*0202 (DR52b) restricted T cell or to a to a DRB1*0101 (DR1) restricted T cell, comprising an amino acid sequence selected from peptides starting at residue 119, 120, 121, 122, 123, 124, 125, 126, or 127 and/or peptides ending at residue 134, 135, 136, 137, 138, 139, 140, 141, 142, or 143 of SEQ ID NO: 1. In some embodiments, the at least one MHC class II molecule of the multimer or the tetramer is loaded with a NY-ESO-1 peptide comprising an amino acid sequence starting at residue 123 and ending at residue 137 of SEQ ID NO: 1. In some embodiments, the method further comprises isolating a cell detected to bind the multimer or tetramer from a population of cells. In some embodiments, the population of cells is a population of blood cells or lymph node cells from a subject. In some embodiments, detecting and/or isolating are performed by fluorescence activated cell sorting (FACS). In some embodiments, the method further comprises quantifying the frequency of multimer-binding or tetramer-binding cells in a population of cells. In some embodiments, the method further comprises comparing the frequency of multimer-binding or tetramer-binding cells in a population of cells obtained from a subject to a control or reference frequency, and if the frequency in the population of cells from the subject is higher than the control or reference frequency, then the subject is indicated to have an immune response to a NY-ESO-1 epitope. In some embodiments, the population of cells is obtained from a subject after an agent or composition has been administered to the subject, the agent or composition comprising an immunostimulatory NY-ESO-1 peptide that is bound by a DRB3*0202 (DR52b) restricted T cell or by a DRB1*0101 (DR1) restricted T cell. In some embodiments, the reference frequency is the frequency observed before the agent or composition comprising an immunostimulatory NY-ESO-1 peptide had been administered to the subject. In some embodiments, the detection agent is a fluorophore suitable for fluorescence activated cell sorting (FACS). In some embodiments, the NY-ESO-1 peptide is fused to a tag. In some embodiments, the tag is a poly-Histidine tag. In some embodiments, the tag comprises between 3 and 12 contiguous histidine residues. In some embodiments, the tag consists of 6 contiguous histidine residues. In some embodiments, the NY-ESO-1 peptide is a fragment of an NY-ESO-1 protein. In some embodiments, the method further comprises obtaining the cell from the subject prior to contacting the cell with the MHC class II multimer or tetramer. In some embodiments, the subject has been administered an immunostimulatory NY-ESO-1 peptide prior to the cell being obtained from the subject. In some embodiments, the method further comprises contacting the cell with an antibody binding an antigen indicative of the differentiation status or cell type of the cell. In some embodiments, the antigen is a surface antigen. In some embodiments, the antigen is indicative of the differentiation status or cell type of a memory T-cell, a helper T cell, or a regulatory T-cell. In some embodiments, the antigen is chosen from the group of CD45RA, CD27, CD28, CCR4, CCR6, CCR7, CXCR3, CD69, CD25, CD127, CRTH2, FOXP3, t-bet, GATA-3, or ROR gamma t. In some embodiments, the antibody is labeled with a detection agent, and the detection agent that the antibody is labeled with is different from the detection agent, if any, that the multimer or tetramer is labeled with. In some embodiments, the antibody is labeled with a fluorophore and the multimer or tetramer is labeled with a fluorophore different from the fluorophore the antibody is labeled with. In some embodiments, the method further comprised detecting both fluorophores on the surface of a cell. In some embodiments, the method further comprised isolating a cell on the surface of which both fluorophores are detected. In some embodiments, the detecting is performed by FACS. In some embodiments, the method further comprises isolating the cell if it is indicated to be a memory T-cell, helper T-cell, or regulatory T-cell. In some embodiments, the method further comprises determining a cytokine profile of the isolated cell. In some embodiments, determining the cytokine profile comprises detecting one or more cytokines produced by the isolated cell. In some embodiments, the one or more cytokine is chosen from the group consisting of IFN-γ, TNF-α, TGF-β, IL-2, IL-4, IL-5, IL-8, IL-9, IL-10, IL-13, IL 17, IL21, IL-22, IP10, MIP-1alpha, MIP-1beta.

In some embodiments, a method of measuring an immune response, for example, an immune response to a vaccination, is provided, comprising obtaining a biological sample comprising a T-cell from a subject, wherein the subject has been administered a composition comprising an epitope of an antigen, contacting the T-cell with an MHC class II multimer, the MHC class II multimer comprising a plurality of MHC class II molecules, wherein at least one of the plurality of MHC class II molecules is loaded with a tagged peptide comprising an epitope of the antigen, and detecting binding of the MHC class II multimer to the T-cell. In some embodiments, the MHC class II multimer is labeled with a detection agent. In some embodiments, the detection agent is a fluorophore. In some embodiments, the method further comprises contacting the T-cell with an antibody binding to a marker indicative of T-cell differentiation status or cell type. In some embodiments, the antigen is chosen from the group of CD45RA, CD27, CD28, CCR4, CCR6, CCR7, CXCR3, CD69, CD25, CD127, CRTH2, FOXP3, t-bet, GATA-3, or ROR gamma t. In some embodiments, the frequency of effector cells (CCR7⁺), “reservoir” memory cells, including central memory (CCR7⁺) and transitional memory (CCR7⁻CD27⁺) T-cells, and/or CD25⁺CD127⁻ Treg cells is determined. In some embodiments, the antibody is labeled with a detection agent, and wherein the detection agent that the antibody is labeled with is different from the detection agent, if any, that the multimer is labeled with. In some embodiments, the antibody is labeled with a fluorophore and the multimer is labeled with a fluorophore different from the fluorophore the antibody is labeled with. In some embodiments, the method further comprises detecting the fluorophores bound to the cell, for example, on the surface of a cell. In some embodiments, the method further comprising isolating a T-cell from the sample based on the detection of the MHC class II multimer binding to the T-cell and, optionally, the presence or absence of the antibody. In some embodiments, the detecting is performed by FACS. In some embodiments, the method further comprises determining a cytokine profile of the isolated T-cell. In some embodiments, determining the cytokine profile comprises detecting one or more cytokines produced by the isolated T-cell. In some embodiments, the one or more cytokine is chosen from the group consisting of IFN-γ, TNF-α, TGF-β, IL-2, IL-4, IL-5, IL-8, IL-9, IL-10, IL-13, IL 17, IL21, IL-22, IP10, MIP-1alpha, MIP-1 beta. In some embodiments, the frequency of IFN-γ⁺ IL-4^{low}IL-17^{low}IL-10⁻TH1 cells, and/or polyfunctional TNF-α⁺IL-2⁺ IFN-γ⁺ CD4⁺ T-cells is determined. In some embodiments, the T-cell is part of a population of cells comprised in the biological sample, and wherein the method further comprises quantifying the T-cells, and/or T-cell subtypes detected in the biological sample. In some embodiments, the method further comprises obtaining a biological sample from an additional subject that has been administered a composition comprising an epitope of the antigen, and comparing the quantities of the T-cells and/or T-cell subtypes detected in the sample from the additional subject to those detected in the sample from the subject. In some embodiments, the epitope administered to the additional subject has not been administered to the subject. In some embodiments, the method further comprises vaccinating further subjects with a composition comprising the epitope that elicited the highest quantities of T-cells or cells of a T-cell subtype among the subjects.

In some embodiments, an isolated immunostimulatory NY-ESO-1 peptide is provided that can specifically bind an MHC class II molecule, and that, when bound to a MHC class II molecule, can specifically bind to a DRB3*0202 (DR52b) restricted CD4⁺ T cell or to a DRB1*0101 (DR1) restricted T cell, the NY-ESO-1 peptide comprising at least 9 contiguous amino acids of NY-ESO-1 (SEQ ID NO: 1). In some embodiments, an isolated immunostimulatory NY-ESO-1 peptide is provided that can specifically bind an MHC class II molecule and that, when bound to a MHC class II molecule, can specifically bind to a DRB3*0202 (DR52b) restricted CD4⁺ T cell or to a DRB1*0101 (DR1) restricted T cell, the peptide comprising an amino acid sequence selected from the group consisting of peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1. In some embodiments, the NY-ESO-1 peptide comprises an amino acid sequence starting at residue 123 and ending at residue 137 of SEQ ID NO: 1. In some embodiments, the NY-ESO-1 peptide comprises the sequence TVSGNILTI (SEQ ID NO: 59). In some embodiments, the NY-ESO-1 peptide comprises the sequence EFTVSGNILTI (SEQ ID NO: 60). In some embodiments, an isolated peptide-loaded DRB3*0202 (DR52b) molecule comprising an immunostimulatory NY-ESO-1 peptide is provided that is bound to a DRB3*0202 (DR52b) molecule. In some embodiments, an isolated peptide-loaded DRB1*0101 (DR1) molecule comprising an immunostimulatory NY-ESO-1 peptide is provided that is bound to a DRB1*0101 (DR1) molecule. In some embodiments, an isolated peptide polytope is provided, comprising an NY-ESO-1 peptide, as described herein, and at least one additional DR52b or DR1 restricted tumor antigen epitope. In some embodiments, the at least one additional DR52b or DR1 restricted tumor antigen epitope is a NY-ESO-1, SSX-4 and/or a Melan-A epitope. In some embodiments, the NY-ESO-1 peptide and/or at least one additional DR52b or DR1 restricted tumor antigen epitope is fused to a tag. In some embodiments, the tag is a His tag. In some embodiments, the tag comprises between 3 and 12 contiguous Histidine residues. In some embodiments, the NY-ESO-1 peptide is a fragment of an NY-ESO-1 protein.

In some embodiments, a method is provided, comprising contacting an isolated MHC molecule with an isolated MHC molecule-binding peptide, wherein the MHC-binding peptide is fused to a tag, thus generating a peptide-loaded MHC molecule, and isolating the peptide-loaded MHC molecule. In some embodiments, the method further comprises linking the MHC molecule to a ligand of a multivalent binding molecule, and contacting the MHC molecule linked to the ligand with the multivalent binding molecule. In some embodiments, a method is provided, comprising contacting an isolated MHC molecule linked to a ligand of a multivalent binding molecule with an isolated MHC-binding peptide, wherein the MHC molecule-binding peptide is fused to a tag, thus generating a peptide-loaded MHC molecule, and isolating the peptide-loaded MHC molecule. In some embodiments, the method further comprises contacting the HLA molecule linked to the ligand with the multivalent binding molecule. In some embodiments, the tag is a peptide or protein tag. In some embodiments, the peptide or protein tag is a tag chosen from the group including a BCCP tag, a myc-tag, a calmodulin-tag, a FLAG-tag, a HA-tag, a His-tag, a maltose binding protein-tag, a nus-tag, a glutathione-S-transferase-tag, a green fluorescent protein-tag, a thioredoxin-tag, a S-tag, a Softag 1, a Softag 3, a strep-tag, a biotin ligase tag, a FlAsH tag, a V5 tag, or a SBP-tag. In some embodiments, the tag is a His tag. In some embodiments, the tag comprises between 3 and 12 contiguous histidine residues. In some embodiments, the His tag consists of 6 contiguous histidine residues. In some embodiments, isolating the complex comprising the MHC molecule bound to the MHC molecule-binding peptide is achieved by affinity chromatography. In some embodiments, the affinity chromatography is Ni²⁺ affinity chromatography. In some embodiments, isolating the MHC molecule contacted with the MHC-molecule binding peptide is achieved by gel filtration chromatography. In some embodiments, the resin employed in the gel filtration has a separation range (Mr) of about 10000 to about 600000. In some embodiments, the resin employed in the gel filtration chromatography is 5200 resin. In some embodiments, the isolated MHC molecule is a MHC class II molecule. In some embodiments, the isolated MHC-binding peptide is an immunostimulatory NY-ESO-1 peptide. In some embodiments, the immunostimulatory NY-ESO-1 peptide can specifically bind an MHC class II molecule, and, when bound to a MHC class II molecule, can specifically bind to a DRB3*0202 (DR52b) restricted CD4⁺ T cell or to a DRB1*0101 (DR1) restricted CD4⁺ T cell. In some embodiments, the immunostimulatory NY-ESO-1 peptide comprises at least 9 contiguous amino acids of NY-ESO-1 (SEQ ID NO: 1). In some embodiments, the immunostimulatory NY-ESO-1 peptide comprises an amino acid sequence selected from the group consisting of peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1. In some embodiments, the immunostimulatory NY-ESO-1 peptide comprises an amino acid sequence starting at residue 123 and ending at residue 137 of SEQ ID NO: 1. In some embodiments, the immunostimulatory NY-ESO-1 peptide comprises the sequence TVSGNILTI (SEQ ID NO: 59). In some embodiments, the immunostimulatory NY-ESO-1 peptide comprises the sequence EFTVSGNILTI (SEQ ID NO: 60). In some embodiments, the MHC molecule comprises a DRB3*0202 (DR52b) molecule or a DRB1*0101 (DR1) molecule.

In some embodiments, a method is provided, comprising obtaining a plurality of MHC class II molecules loaded with peptides of different sequences, wherein the peptide sequences comprise 9 contiguous amino acids of a protein antigen, contacting a T-cell or a T-cell receptor with the plurality of peptide-loaded MHC class II molecules, and detecting a binding event between the T-cell or T-cell receptor and a peptide-loaded MHC class II molecule comprised in the plurality of MHC class II molecules, wherein a peptide-loaded MHC class II molecule determined to bind the T-cell or T-cell receptor is identified as comprising an epitope of the antigen. In some embodiments, the method further comprises determining the amino acid sequence of the peptide comprised in the peptide-loaded MHC class II molecule determined to bind the T-cell or T-cell receptor. In some embodiments, the peptides are fused to a tag. In some embodiments, tag is a peptide or protein tag. In some embodiments, the peptide or protein tag is a tag chosen from the group including a BCCP tag, a myc-tag, a calmodulin-tag, a FLAG-tag, a HA-tag, a His-tag, a maltose binding protein-tag, a nus-tag, a glutathione-S-transferase-tag, a green fluorescent protein-tag, a thioredoxin-tag, a S-tag, a Softag 1, a Softag 3, a strep-tag, a biotin ligase tag, a FlAsH tag, a V5 tag, or a SBP-tag. In some embodiments, the tag is a His tag. In some embodiments, the tag comprises between 3 and 12 contiguous histidine residues. In some embodiments, the His tag consists of 6 contiguous histidine residues; In some embodiments, the MHC class II molecules are comprised in MHC class II multimers. In some embodiments, the MHC class II multimers are MHC class II tetramers. In some embodiments, each multimer or tetramer comprises peptides of the same sequence. In some embodiments, the T-cell receptor is expressed by a T-cell. In some embodiments, the T-cell is obtained from a subject. In some embodiments, the subject is diagnosed with having a tumor and the protein antigen is a protein antigen expressed by the tumor. In some embodiments, the T-cell is obtained from the subject after vaccination of the subject with the protein antigen, or a fragment thereof. In some embodiments, the contacting is performed in vivo, in vitro, or ex vivo. In some embodiments, the T-cell receptor is isolated prior to contacting. In some embodiments, the T-cell receptor or the plurality of MHC class II molecules is immobilized on a solid support. In some embodiments, the T-cell receptor is contacted with a composition comprising two or more of the plurality of MHC class II molecules. In some embodiments, the peptide-loaded MHC class II molecules are labeled with a detection agent in a manner allowing for the identification of the peptide comprised in a specific MHC class II molecule. In some embodiments, the T-cell receptor is contacted with a composition comprising one of the plurality of MHC class II molecules. In some embodiments, a plurality of steps of contacting the T-cell receptor with a composition comprising one of the plurality of MHC class II molecules is performed in parallel or sequentially. In some embodiments, the peptides comprise sequences of 20-40 contiguous amino acids. In some embodiments, the peptides comprise overlapping sequences of contiguous amino acid sequences of the antigen.

In some embodiments, a kit is provided, comprising an isolated MHC class II molecule, multimer, or tetramer as described herein, and/or an isolated immunostimulatory NY-ESO-1 peptide as described herein. In some embodiments, the kit further comprises a detectable label, a labeling reagent, a detection reagent, and/or a buffering reagent. In some embodiments, the kit further comprised an antibody to a marker indicative of T-cell differentiation status and/or T-cell subtype.

Some aspects of this invention provide tagged MHC class II binding peptides or methods using such peptides. Such tags are useful for the isolation of the tagged peptide, either alone or when bound to an MHC class II molecule. Methods for isolating tagged peptides are well known to those of skill in the art and include, for example, affinity chromatography methods. In some embodiments, an MHC class II binding peptide is provided or used that is conjugated to a tag. In some embodiments, the tag is a peptide tag. In some embodiments, the tag is a poly-Histidine tag. In some embodiments, the tag comprises 3-12 histidine residues. In some embodiments, the tag consists of 6 contiguous histidine residues.

Some aspect of this invention relate to improved methods for detecting T cells specifically binding an antigenic peptide loaded onto an MHC molecule.

Some aspects of this invention provide an isolated DR MHC class II molecule. In some embodiments, the MHC class II molecule comprises a DR beta chain and a DR alpha chain. In some embodiments, the beta chain comprises an extracellular part of a DR52b protein fused to a leucine zipper sequence. In some embodiments, the alpha chain comprises an extracellular part of a DR MHC class II alpha chain fused to a leucine zipper sequence that binds to the leucine zipper sequence fused to the beta chain. In some embodiments, the MHC class II molecule comprises an alpha chain comprising the amino acid sequence provided in SEQ ID NO: 79. In some embodiments, the MHC class II molecule comprises a beta chain comprising the amino acid sequence provided in SEQ ID NO: 81. In some embodiments, the MHC class II molecule comprises an alpha chain comprising the amino acid sequence provided in SEQ ID NO: 79, and a beta chain comprising the amino acid sequence provided in SEQ ID NO: 81. In some embodiments, the leucine zipper fused to the DR alpha chain is an acidic leucine zipper. In some embodiments, the leucine zipper fused to the beta chain is a basic leucine zipper. In some embodiments, the leucine zipper is followed by an Avi-Tag (also called BSP sequence). In some embodiments, the leucine zippers are fused to the MHC class II alpha and/or beta chains via a glycine-serine linker. In some embodiments, the MHC class II molecule is not loaded with an antigenic peptide. In some embodiments, the MHC class II molecule is loaded with an antigenic peptide. In some embodiments, the MHC class II molecule is loaded with an NY-ESO-1 antigenic peptide, for example, an NY-ESO-1 antigenic peptide as described herein.

These and other aspects of the invention, as well as various advantages and utilities will be more apparent with reference to the drawings and detailed description of the invention.

### BRIEF DESCRIPTION OF THE DRAWINGS

FIG. 1. Isolation of ESO₁₁₉₋₁₄₃-specific CD4⁺ T cell clones. A, Pre- and Post-vaccine samples from patient C2 were analyzed ex-vivo for the presence of ESO-specific CD4⁺ T cells by intracellular IFN-γ staining following stimulation in the absence or presence of the ESO peptide pool. Numbers are % IFN-γ⁺ cells among CD4⁺ T cells. B, ESO-specific clones derived from patient C2 were stimulated in the absence or presence of the indicated peptides (2 μM) and IFN-γ production was assessed by intracellular cytokine staining. C, ESO-specific clones were stimulated in the presence of graded concentrations of the indicated peptides and IFN-γ was measured in the culture supernatant by ELISA. D, TCR BV usage of ESO₁₁₉₋₁₄₃-specific CD4⁺ T clones was assessed by staining with a panel of anti-BV mAb and flow cytometry analysis. Results are shown for one clone representative of four (B, C, D).

FIG. 2. MHC class II restriction of ESO₁₁₉₋₁₄₃-specific CD4⁺ T cell clones. A, Clones were stimulated with peptide ESO₁₁₉₋₁₄₃ (2 μM) in the absence or presence of anti-DR, -DP or -DQ mAb and IFN-γ production was assessed by intracellular cytokine staining. Histograms for one representative clone and a summary of results for four clones from patient C2 are shown. Numbers in histograms correspond to mean fluorescence intensity (MFI) of IFN-γ staining. % inhibition=100−((MFI IFN-γ in presence of anti-HLA mAb/MFI IFN-γ in absence of mAb)×100). B, Clone C2/C4E7 was stimulated in the presence of PMBC from 15 HD, that were pre-incubated in the absence or presence of peptide ESO₁₁₉₋₁₄₃, and IFN-γ production was assessed by intracellular cytokine staining. C, Clones C2/C4E7 and 672/33 were stimulated with the indicated peptides in the absence or presence of anti-DR or -DR52 mAb and IFN-γ was measured in culture supernatants by ELISA. D, Clone C2/C4E7 was incubated with indicated molecularly typed B-EBV cell lines, that were pre-incubated in the absence or presence of peptide ESO₁₁₉₋₁₄₃, and IFN-γ was measured in culture supernatants by ELISA.

FIG. 3. Determination of the minimal sequence optimally recognized by ESO₁₁₉₋₁₄₃-specific CD4⁺ T cell clones. Clone C2/C4E7 was stimulated, in the presence of EBV14, with serial dilutions of the indicated truncated peptides, IFN-γ was measured in culture supernatants by ELISA (upper panel) and peptide activity was calculated relative to that of ESO₁₁₉₋₁₄₃ (lower panel).

FIG. 4. Recognition of naturally processed ESO protein by DR52b-restricted CD4⁺ T cells. A, Surface expression of DR52 on DR52b⁺ moDC was assessed by staining with specific mAb and flow cytometry analysis (left panel). Clone C2/C4E7 was stimulated with moDC pulsed with ESO or Melan-A recombinant proteins at the indicated concentrations and IFN-γ was measured in culture supernatants by ELISA (right panel). B, Surface expression of DR52 on DR52b⁺ tumor lines was assessed as in A, following 24 h culture in the absence or presence of IFN-γ (500 IU/ml). C, Tumor cell lines, cultured in the absence or presence of IFN-γ for 24 h, were pre-incubated in the absence or presence of peptide ESO₁₁₉₋₁₄₃ and used to stimulated clone C2/C4E7. IFN-γ was then measured in culture supernatants by ELISA. D, A2⁺DR52b⁺ moDC were transfected with full-length ESO encoding plasmid or incubated with the indicated peptide and used to stimulate DR52b- or A2-restricted clones. IFN-γ was measured in 24 h culture supernatants by ELISA.

FIG. 5. Induction of DR52b-restricted ESO₁₁₉₋₁₄₃-specific CD4⁺ T cell responses following vaccination with ESO protein. A, % of IFN-γ-producing CD4⁺ T cells in response to peptide ESO₁₁₉₋₁₄₃ in post-vaccine cultures from DR52b⁺ and DR52b⁻ patients assessed by intracellular cytokine staining. B, % IFN-γ-producing CD4⁺ T cells in the same cultures as in A was assessed in the absence or presence of anti-DR52 mAb. % inhibition=100−((% IFN-γ⁺ CD4⁺ T cells in presence of mAb/% IFN-γ⁺ CD4⁺ T cells in absence of mAb)×100). Mean % inhibition for all DR52b⁺ and DR52b⁻ patients is shown.

FIG. 6. Molecularly defined DR52b/ESO₁₂₃₋₁₃₇ tetramers stain specific CD4⁺ T cell clones. (A and B) ESO-specific DR52b-restricted and control clonal populations were stained with DR52b/ESO₁₂₃₋₁₃₇ tetramers, at the indicated concentrations, for 1 hr at 37° C. followed by staining with anti-CD4 mAb and flow cytometry analysis. Examples of dot plots for both populations are shown in A and the mean fluorescence intensity (MFI) of tetramer staining for all concentrations is summarized in B. (C) ESO specific and control clonal populations were stained with DR52b/ESO₁₂₃₋₁₃₇ tetramers (3 μg/ml) at 4° C., 23° C. or 37° C. for the indicated periods and analyzed as in A. (D) ESO specific clonal cells were stained with DR52b/ESO₁₂₃₋₁₃₇ tetramers (3 μg/ml) for 1 hr at 37° C., extensively washed and further incubated at 4° C., 23° C. or 37° C. for the indicated periods prior to flow cytometry analysis.

FIG. 7. DR52b/ESO₁₂₃₋₁₃₇ tetramers stain peptide-stimulated post-vaccine CD4⁺ T cell cultures from DR52b⁺ patients. (A) Post-vaccine CD4⁺ T cells from DR52b⁺ and DR52b⁻ patients stimulated in vitro with a pool of overlapping long ESO peptides were stained with DR52b/ESO₁₂₃₋₁₃₇ tetramers (3 μg/ml) for 1 hr at 37° C. and anti-CD4 mAb and analyzed by flow cytometry. Dot plots for 2 patients and data for all patients tested are shown. Numbers in dot plots correspond to the percentage of tetramer⁺ cells. (B) Peptide stimulated CD4⁺ T cells were stained with tetramers for the indicated time periods and analyzed by flow cytometry. Numbers in dot plots correspond to the percentage of tetramer⁺ cells and numbers between brackets indicate the MFI of tetramer staining of the tetramer⁺ population. Results are shown for one patient (C05) representative of three tested.

FIG. 8. DR52/ESO₁₁₉₋₁₄₃ and DR52b/ESO₁₂₃₋₁₃₇ tetramers stain specific clones as well as peptide-stimulated CD4⁺ T cells from post-vaccine but not from pre-vaccine samples. (A) ESO specific DR52b restricted and control clonal populations were stained with DR52b/ESO₁₂₃₋₁₃₇ or DR52b/ESO₁₁₉₋₁₄₃ tetramers, at the indicated concentrations, for 1 hr at 37° C. followed by staining with anti-CD4 mAb and flow cytometry analysis. Dot plots of an ESO-specific clone stained with both tetramers at 3 μg/ml and MFI of tetramer staining at all concentrations tested are shown. (B) Post-vaccine peptide stimulated CD4⁺ T cells from DR52b⁺ patients were stained with DR52b/ESO₁₁₉₋₁₄₃ or DR52b/ESO₁₂₃₋₁₃₇ tetramers (3 μg/ml) for 1 hr at 37° C. and analyzed by flow cytometry. Dot plots for patient C06 and data for all patients tested are shown. Numbers in dot plots correspond to the percentage of tetramer⁺ cells. (C) Peptide-stimulated CD4⁺ T cells from pre-vaccine and post-vaccine samples from DR52b⁺ patients C02 and N10 were stained and analyzed as in B.

FIG. 9. DR52b/ESO tetramers allow direct ex vivo quantification of specific vaccine-induced CD4⁺ T cells. CD4⁺ T cells purified from PBMC from DR52b⁺ healthy donors (HD) and from pre- and post-vaccine samples from DR52b⁺ patients were stained ex vivo with DR52b/ESO₁₁₉₋₁₄₃ tetramers (3 μg/ml) during 2 hrs at 37° C. and were then stained with anti-CD45RA mAb and analyzed by flow cytometry. Dot plots for one HD, one pre-vaccine sample and all post-vaccine samples are shown in A and data for all samples tested are summarized in B. Numbers in dot plots correspond to the percentage of tetramer⁺ cells among memory CD45RA⁻ CD4⁺ T cells.

FIG. 10. DR52b/ESO tetramers allow ex vivo phenotyping of specific vaccine-induced CD4⁺ T cells. (A and B) Post-vaccine CD4⁺ T cells from DR52b⁺ patients were stained ex vivo with DR52b/ESO₁₁₉₋₁₄₃ tetramers as in FIG. 4 as well as with CD45RA, CCR7, CD27 and CD28 specific mAb. Dot plots for patient N13 are shown gated on tetramer⁻ (upper panel) and tetramer⁺ (lower panel) cells in A and data corresponding to the percentage of central memory (CM, CD45RA⁻CCR7⁺), transitional memory (TM, CD45RA⁻ CCR7⁻CD27⁺) and effector memory (EM, CD45RA⁻CCR7⁻CD27⁻) cells among tetramer⁺ cells for all patients are summarized in B. (C and D) Samples were stained with DR52b/ESO₁₁₉₋₁₄₃ tetramers as in A as well as with CD45RA, CD25 and CD127 specific mAb. Dot plots for patient C02 are shown gated on memory tetramer⁺ and tetramer⁺ cells in C and data obtained for all patients are summarized in D.

FIG. 11. DR52b/ESO tetramers allow the isolation and functional characterization of specific CD4⁺ T cells. (A) Peptide-stimulated post-vaccine samples were stained with tetramers (left panel) and tetramer⁺ and tetramer⁻ cells were isolated by flow cytometry cell sorting. Aliquots of sorted cells were directly re-analyzed by flow cytometry (middle panels). Tetramer⁺ cells were expanded in vitro and the purity of the resulting polyclonal populations was assessed by flow cytometry analysis following tetramer staining (right panel). Numbers correspond to the percentage of tetramer⁺ cells. Results are shown for one patient and are representative of data obtained for 4 patients. (B and C) ESO specific polyclonal cultures obtained in A were stimulated with PMA and ionomycin and cytokine production was assessed in a standard 4 hrs intracellular cytokine assay and flow cytometry analysis. Dot plots are shown for one patient and are representative of data obtained for 4 patients. (D) ESO specific polyclonal cultures were incubated either with DR52b⁺ EBV-B cells and ESO₁₁₉₋₁₄₃ or control peptide (left panel) or with DR52b⁺ monocyte-derived dendritic cells pre-incubated with rESO or control protein (right panel), at the indicated concentrations, and IFN-γ was measured by ELISA in 24 hrs culture supernatants.

FIG. 12. DR52b/ESO tetramer staining allows the direct assessment of TCR Vβ usage by specific CD4⁺ T cells. Peptide-stimulated post-vaccine CD4⁺ T cells were first stained with DR52b/ESO tetramers and then with a panel of anti-TCR Vβ mAB and analyzed by flow cytometry. Dot plots obtained with anti-Vβ2 and anti-Vβ5.1 mAb are shown for one patient in A and data showing the percentage of Vβ2⁺ cells among tetramer⁺ cells for all patients are summarized in B.

FIG. 13. Classical DR52b tetramers containing peptide ESO₁₂₃₋₁₃₇ fail to stain specific clonal populations. (A) ESO-specific DR52b-restricted and control clonal populations were stimulated in the absence or presence of ESO peptide and IFN-γ production was assessed in a standard intracellular staining assay and flow cytometry analysis. (B) ESO-specific and control clonal populations were stained with DR52b/ESO₁₂₃₋₁₃₇ tetramers (3 μg/ml) for 1 hr at 37° C. and then with anti-CD4 mAb and analyzed by flow cytometry. Numbers correspond to the percentage of tetramer⁺ CD4⁺ cells.

FIG. 14. Schematic presentation of key steps for the production of molecularly defined DR52b tetramers containing His-tag peptides allowing the purification of pMHC molecules by affinity chromatography prior to gel filtration chromatography and tetramerization in the presence of phycoerythrin-labeled streptavidin (SA-PE).

FIG. 15. The purity of the isolated biotinylated monomers was assessed in a shift assay with avidin.

FIG. 16. DR52b binding capacity of His-tagged and untagged ESO peptides and recognition by specific clones. (A) The capacity of His-tagged and untagged ESO peptides to bind to DR52b was assessed in a competition assay with a biotin-labeled HA-derived peptide. ESO and control peptides were incubated overnight at 37° C., at the indicated concentrations, with recombinant empty DR52b protein (5 μg) and biotin-labeled HA₃₀₆₋₃₁₈ peptide (0.2 μM). The quantity of DR52b molecules containing the biotin-labeled peptide in each test-point was assessed by ELISA using anti-HLA-DR (clone L243) coated plates and alkaline phosphatase-labeled streptavidin. (B) ESO specific clones were incubated with DR52b⁺ EBV-B cells and ESO peptides, at the indicated concentrations, and IFN-γ was measured by ELISA in 24 hrs culture supernatants. Results are shown for one clone representative of 3 clones tested.

FIG. 17. DR1/ESO119-143 tetramers stain ESO119-143-specific DR1-restricted CD4 T cell clones. A, ESO119-143-specific clonal populations from DR1+ patient N03 were incubated with untransfected or DR1-expressing mouse fibroblasts that had been pulsed or not with peptide ESO119-143 and IFN-γ production was assessed in a 4 hr intracellular cytokine staining assay. B, ESO-specific DR1-restricted and control clonal populations were stained with serial dilutions of DR1/His-ESO119-143 tetramers for 1 hr at 37° C. followed by staining with anti-CD4 mAb and flow cytometry analysis. Examples of dot plots for the ESO-specific clone and the mean fluorescence intensity (MFI) of tetramer staining for both clones at all concentrations are shown. C, ESO-specific DR1-restricted and control clonal populations were stained with DR1/His-ESO119-143 tetramers (3 μg/ml) at 4° C., 23° C. or 37° C. for the indicated periods and analyzed as in B. D, ESO-specific DR1-restricted and control clonal populations were stained with DR1 tetramers containing untagged or His-tagged ESO119-143 peptides and analyzed as in B. Examples of dot plots for staining of ESO-specific cells with both tetramers at 10 μg/ml and MFI of tetramer staining for all conditions are shown.

FIG. 18. DR1/ESO119-143 tetramers stain peptide-stimulated CD4 T cells from post-vaccine but not from pre-vaccine samples of DR1+ patients. A, Post-vaccine CD4 T cells from DR1+ patient N03, stimulated in vitro with a pool of overlapping long ESO peptides spanning the full-length ESO sequence, were stained with DR1/ESO119-143 or control DR1/ESO95-106 tetramers (3 μg/ml) for 1 hr at 37° C. and anti-CD4 mAb and analyzed by flow cytometry. B, Pre- and post-vaccine CD4 T cells from DR1+ patients, stimulated in vitro with peptide ESO119-143, were stained with DR1/ESO119-143 tetramers and anti-CD4 mAb and analyzed by flow cytometry. Dot plots for patient N11 and data for all patients are shown.

FIG. 19. Isolation and functional characterization of vaccine-induced DR1/ESO119-143 tetramer+ CD4 T cells. A, Post-vaccine CD4 T cells were stimulated in vitro with peptide ESO119-143, stained with DR1/ESO119-143 tetramers (left dot plot) and tetramer+ and tetramer-cells were isolated by flow cytometry cell sorting. Aliquots of sorted cells were directly re-analyzed by flow cytometry (middle dot plots). Tetramer+ cells were expanded in vitro and the purity of the resulting polyclonal populations was assessed by flow cytometry analysis following tetramer staining (right dot plot). Polyclonal populations were also incubated with L.DR1 cells and serial dilutions of ESO119-143 or control peptide and IFN-γ was measured by ELISA in 24 hrs culture supernatants. Results are shown for one patient, N03, representative of four. B, Tetramer+ polyclonal populations were incubated with L.DR1 cells, that have been pulsed or not with peptide ESO119-143, and IFN-γ production was assessed in a 4 hr intracellular cytokine staining assay. C, Tetramer+ polyclonal populations were incubated either with L.DR1 cells and serial dilutions of ESO119-143 or control peptide (left panel) or with DR1+ monocyte-derived dendritic cells pre-incubated with serial dilutions of rESO or control protein (middle panel) and IFN-γ was measured by ELISA in 24 hrs culture supernatants. Examples of peptide and protein recognition are shown for patient N11 and the concentration of peptide and protein resulting in half maximal IFN-γ secretion (EC50) is shown for all patients. D, Polyclonal cultures were stimulated with PMA and ionomycin and cytokine production was assessed in a 4 hr intracellular cytokine assay. Examples of dot plots for patient N03 and data obtained for all patients and all cytokines tested are shown.

FIG. 20. Assessment of TCR Vβ usage by Vaccine-induced ESO119-143-specific DR1-restricted CD4 T cells. Polyclonal monospecific tetramer+ populations from vaccinated patients were first stained with DR1/ESO119-143 tetramers and then with a panel of anti-TCR Vβ mAb and analyzed by flow cytometry. Examples of dot plots obtained with anti-Vu 1 and anti-Vβ2 mAb staining for patient N03 are shown in A. Numbers correspond to the percentage of Vβ+ cells among tetramer+ cells in the culture. Results corresponding to the percentage of Vβ+ cells, for all Vβ tested, among tetramer+ cells for all patients are summarized in B.

FIG. 21. Assessment of the minimal peptide optimally recognized by vaccine-induced ESOspecific DR1-restricted CD4 T cells. A, Polyclonal DR1/ESO119-143 tetramer+ cultures, obtained as in FIG. 28A, from patient N03 were incubated with L.DR1 cells and serial dilutions of truncated peptides within the 119-143 region or ESO1-20 control peptide and IFN-γ was measured by ELISA in 24 hrs culture supernatants (examples are shown in the left panel). The activity of each peptide (EC50) was calculated relative to that of peptide ESO119-143 (right panel). B, ESO-specific DR1-restricted or control clonal populations were stained with serial dilutions of DR1 tetramers containing peptides ESO119-143 or ESO123-137 and analyzed by flow cytometry as in FIG. 26B. C, Post-vaccine CD4 T cells from DR1+ patients were stimulated in vitro with peptide ESO119-143, stained with DR1/ESO119-143 or DR1/ESO123-137 tetramers and anti-CD4 mAb and analyzed by flow cytometry. Dot plots for patient C04 and data for all patients are shown.

FIG. 22. Ex vivo assessment of vaccine-induced ESO-specific DR1-restricted CD4 T cells. CD4 T cells purified from PBMC from pre- and post-vaccine samples of DR1+ patients were stained ex vivo with DR1/ESO119-143 tetramers (3 μg/ml) during 2 hrs at 37° C. and were then stained with anti-CD4, -CD45RA and -CCR7 mAb and analyzed by flow cytometry. A, Examples of dot plots for pre- and post-vaccine samples. Numbers in dot plots correspond to the percentage of tetramer+ cells among memory CD45RA− CD4 T cells. B, Percentage of tetramer+ cells among memory CD45RA− CD4 T cells in pre-vaccine and post-vaccine samples (PV 3, one week following the 3rd vaccine injection; PV 4, one week following the 4th vaccine injection; PT, 4 to 5 months following the 4th and last vaccine injection). C, Phenotype of tetramer+ cells in PV 3 samples based on CD45RA and CCR7 staining (CM, central memory CD45RA−CCR7+; EM, effector memory CD45RA−CCR7−).

### DETAILED DESCRIPTION

Identification of immunodominant tumor antigen-derived CD4⁺ T cell epitopes restricted by frequently expressed MHC class II molecules is instrumental for the immunological monitoring of tumor antigen-based vaccine trials, allowing for assessment of correlates between immune responses and clinical outcomes. Whereas many CD4⁺ T cell epitopes restricted by MHC-DR molecules encoded by the highly polymorphic DRB1 gene have been characterized, only few epitopes restricted by molecules encoded by the less polymorphic DRB3, DRB4 or DRB5 genes have been identified thus far. Here, we have characterized CD4⁺ T cell responses induced by vaccination with a recombinant NY-ESO-1 (rESO) protein and identified an ESO-derived, DR52b (DRB3*0202)-restricted, CD4⁺ T cell epitope. The identified epitope is immunodominant, as specific responses were detectable in all vaccinated patients expressing DR52b, and is recognized by CD4⁺ T cells exhibiting conserved TCR usage in different individuals.

NY-ESO-1, also referred to herein as ESO, is a tumor-specific antigen with wide expression in human tumors of different histological types and remarkable spontaneous immunogenicity. We have previously shown that specific T_{H}1 and antibody responses can be elicited in patients with no detectable pre-existing immune responses by vaccination with rESO administered with Montanide ISA-51 and CpG ODN 7909. The purpose of the present study was to characterize vaccine-induced ESO-specific CD4⁺ T cell responses.

We generated CD4⁺ T cell clones from patient C2, who had the highest CD4⁺ T cell response to the vaccine, and analyzed their fine specificity and MHC class II restriction to determine the recognized epitope. We then assessed the response to the identified epitope in all vaccinated patients expressing the corresponding MHC class II allele.

We found that ESO-specific CD4⁺ T cell clones from patient C2 recognize peptide ESO₁₁₉₋₁₄₃ (core region 123-137) presented by HLA-DR52b (HLA-DRB3*0202), an MHC class II allele expressed by about half of Caucasians. Importantly, following vaccination, all patients expressing DR52b developed significant responses to the identified epitope, accounting for, in average, half of the total CD4⁺ T cell responses to the 119-143 immunodominant region. In addition, analysis of ESO-specific DR52b-restricted CD4⁺ T cells at the clonal level revealed significant conservation of TCR usage among different individuals.

The identification of a DR52b-restricted epitope from ESO that is immunodominant in the context of vaccine-elicited, immune responses is instrumental for the immunological monitoring of vaccination trials targeting this important tumor antigen. By assessing vaccine-induced CD4⁺ T cells, we have identified an immunodominant epitope (ESO₁₁₉₋₁₄₃, core region ESO₁₂₃₋₁₃₇) restricted by HLA-DR52b (DRB3*0202), an allele expressed by half of Caucasians. DRB3-DRB4- and DRB5-encoded molecules are less polymorphic than those encoded by DRB1 and are therefore attractive candidates for the development of generic MHC class II tetramers.

Soluble MHC-peptide tetramers, allowing the direct visualization, characterization and isolation of antigen-specific T cells, have become essential tools for T cell analysis. MHC class I tetramers incorporating short CTL peptide epitopes, originally developed by J. D. Altman and M. M. Davis have been generated for a large number of murine and human alleles incorporating a variety of peptides of microbial, tumor and self-antigen origin. The development of MHC class II tetramers, however, and particularly of those incorporating peptides from tumor and self-antigens, has been far less successful. One limiting factor is the high polymorphism of the human MHC class II molecules, especially those encoded by the DRB1 locus, the most frequently studied. Another one is the binding affinity of antigenic peptides derived from tumor and self-antigens, which is generally lower than that of peptides from pathogens.

The immunodominant epitope (ESO₁₁₉₋₁₄₃, core region ESO₁₂₃₋₁₃₇) is restricted by HLA-DR52b, a DRB3-encoded molecule. DRB3-DRB4- and DRB5-encoded molecules are less polymorphic than those encoded by DRB1 and are, therefore, attractive candidates for the development of generic MHC class II tetramers. The immunodominant epitope ESO₁₁₉₋₁₄₃ is also restricted by HLA-DR1, a DRB1-encoded molecule. Accordingly, the immunodominant ESO₁₁₉₋₄₄₃ epitope is useful for therapeutic and diagnostic methods in subjects expressing a DRB1 or DR52b allele.

Our initial attempts to construct DR52b/ESO tetramers using an approach previously described by Kwok et al., by peptide loading of class II molecules incorporating “leucine zipper” motifs, failed to generate efficient tetramers. We therefore designed a novel strategy using His-tagged peptides that allows isolation of folded MHC/peptide monomers by affinity purification prior to tetramerization. Tetramers generated according to this procedure avidly and stably bound to ESO-specific CD4⁺ T cells, allowing their direct ex vivo enumeration, phenotyping and isolation from circulating lymphocytes of vaccinated patients. The application of this novel strategy to other tumor and self-antigen derived peptides may significantly accelerate the development of reliable MHC class II tetramers to monitor antigen-specific CD4⁺ T cells.

Some aspects of this invention relate to the surprising discovery that MHC class II binding peptides bind MHC class II molecules in the presence of a tag covalently bound to the binding peptides (a “tagged peptide”), for example, a peptide tag, such as a His tag, with similar affinity to that of untagged peptides. Some aspects of this invention provide a method for generating a MHC class II monomer loaded with a tagged peptide. Some aspects of the invention provide a method for isolating and/or purifying a MHC class II monomer loaded with a tagged peptide. Some aspects of this invention provide a method for generating MHC class II multimers, for example, tetramers, by contacting a MHC class II monomer loaded with a tagged peptide, and linked to a ligand of a multivalent binding molecule, with the multivalent binding molecule.

In some embodiments, a MHC class II monomer loaded with a tagged peptide, for example, a His-tagged peptide, is generated by contacting a MHC class II molecule with a tagged, MHC class II molecule-binding peptide. In some embodiments, the MHC class II molecule loaded with a tagged peptide is isolated and/or purified. In some embodiments, the isolation and/or purification comprises a step of enriching for peptide-loaded MHC-class II molecules comprising the tag, for example, an affinity chromatography step, and/or a step enriching for molecules of a desired size or size range, for example, a size fractionation step.

The surprising discovery that a tag, for example, a peptide tag, does not significantly interfere with the binding of a tagged peptide to an MHC class II molecule, allows for the design of an isolation and/or purification strategy that selects for MHC class II monomers that are loaded with the tagged peptide. This is in contrast to conventional methods in which no tag is used or in which a tag is placed on the MHC molecule itself. While such methods allow for isolation and/or purification of “empty” MHC molecules (e.g., MHC molecules that are not peptide-loaded) by enriching for tag-bearing molecules of a desired size or size range, they typically do not allow a distinction between peptide-loaded and “empty” MHC molecules. Since, generally, only a fraction of MHC class II molecules bind an MHC class II binding peptide during the process of peptide-loading, such conventional methods yield mixtures of empty and peptide-loaded MHC class II molecules. The use of such mixtures, for example, in the preparation of MHC class II tetramers, results in decreased sensitivity, since only a fraction of the MHC class II molecules are peptide-loaded. One of the advantages of the methods provided by some aspects of this invention is that the use of tagged MHC class II binding peptides allows for selection of MHC class II molecules that have bound such a peptide, and, accordingly, for the exclusion of empty MHC class II molecules. In some embodiments, the MHC class II molecules to be loaded with MHC class II binding peptides comprise both correctly folded (“native”) and incorrectly folded (“denatured”) MHC class II molecules. In some embodiments, only the correctly folded MHC class II molecules efficiently bind the MHC class II binding peptides, while the incorrectly folded MHC class II molecules do not. In some embodiments, isolation of peptide-loaded MHC class II molecules from a mixture of incorrectly folded and correctly folded MHC class II molecules contacted with a tagged MHC class II binding peptide by enrichment/purification of molecule complexes comprising the tag and having a desired size or molecular weight allows for the exclusion of unbound peptide and incorrectly folded MHC class II molecules.

In some embodiments, isolated and/or purified MHC class II monomers comprise a ligand of a multivalent binding molecule or are linked to such a ligand after isolation and/or purification. In some embodiments, such MHC class II monomers are contacted with the multivalent binding molecule to generate peptide-loaded MHC class II multimers.

Some aspects of this invention relate to the surprising discovery that multimers, for example, tetramers, of MHC class II molecules loaded with tagged peptides avidly and stably bind target CD4⁺ T-cells. Accordingly, some aspects of this invention provide a method to contact a CD4⁺ T-cell with a MHC class II multimer, for example, a tetramer, and to detect the binding and/or identify the target CD4⁺ T-cell. In some embodiments, the MHC class II multimer (e.g. tetramer) is labeled with a detectable label and a cell to which the multimer binds is identified by detecting the label on the surface of the cell. Detectable labels and methods for the detection of such labels on the surface of cells are well known to those of skill in the art and examples of such labels include, but are not limited to fluorescein and its derivatives (e.g., fluorescein isothiocyanate (FITC)), phycoerythrin (PE), R-phycoerythrin (rPE) PE I, PE II, PE Texas Red, PE-Cy5, Peridinin chlorophyll protein (PerCP), propidium iodide (PI), PerCP/PI, PerCP-Cy5, PE-Cy7, allophycocyanin (APC), APC-Cy7, Alexa fluor 405, Alexa fluor 430, Alexa fluor 488, Alexa fluor 532, Alexa fluor 546, Alexa fluor 555, Alexa fluor 568, Alexa fluor 594, Alexa fluor 633, Alexa fluor 660, Alexa fluor 680, Pacific Blue, rhodamine, aminocoumarin, hydroxycoumarin, methoxycoumarin, HEX, TRITC, Tamara, 7-Aminoactinomycin D (7-AAD), fluorescent proteins (for example, GFP, YFP, BFP, RFP, also enhanced versions, such as eGFP. eYFP etc.), and Cyanine dyes, for example, Cy3 and Cy5. In some embodiments, the detection method is a flow cytometry method, for example a fluorescence-activated cell sorting (FACS) method. Flow cytometry methods and detectable labels for such methods are well known to a person skilled in the relevant art, and non-limiting examples for references disclosing such methods and labels are Zbigniew Darzynkiewicz, Cytometry (Methods in Cell Biology), Academic Press, 4th edition (October 2004), ISBN-10: 0124802834; John L. Carey et al., Flow Cytometry, American Society for Clinical Pathology, 4th edition (Oct. 8, 2007), ISBN-10: 0891895485; both of which are incorporated herein by reference for disclosure of flow cytometric methods and detectable labels.

In some embodiments, labeled, peptide-loaded MHC class II molecules, monomers, or multimers, are used to identify, detect, and/or isolate a CD4+ T-cell specifically binding the peptide loaded onto the MHC class II molecule, monomer, or multimer, from a cell population, for example, a cell population obtained from a patient. In some embodiments, the isolated T-cell is phenotypically and/or functionally characterized, for example, in regard to its cell subtype, in regard to differentiation, activation, adhesion or migration marker expression and/or in regard to its cytokine or chemokine expression or excretion profile. T-cell subtype markers (e.g., CD25, CD127, CRTH2), differentiation, adhesion, activation, and migration markers (e.g., CD45RA, CD27, CD28, CCR4, CCR6, CCR7, CXCR3, CD69), and lineage-specific transcription factors (e.g., FOXP3, t-bet, GATA-3, ROR gamma t) are well known to those of skill in the art. Cytokine and chemokine markers, detectable, for example, by antibodies in flow cytometry or other immunofluorescence-based assays (e.g., IFN-γ, TNF-α, TGF-β, IL-2, IL-4, IL-5, IL-8, IL-9, IL-10, IL-13, IL 17, IL21, IL-22, IP10, MIP-1alpha, MIP-1beta) are also well known to those of skill in the relevant arts.

The term “MHC” refers to the major histocompatibility complex. In humans, the term MHC is used interchangeably with the term “HLA” (human leukocyte antigen).

The term “NY-ESO-1” is interchangeably used with the terms “ESO” and “ESO-1” herein. NY-ESO-1 is also known as “cancer/testis antigen 1B”. A representative sequence of human NY-ESO-1 protein is given below:

>gi|4503119|ref|NP_001318.1|cancer/testis antigen
1B):
(SEQ ID NO: 1)
MQAEGRGTGGSTGDADGPGGPGIPDGPGGNAGGPGEAGATGGRGPRGAGA
ARASGPGGGAPRGPHGGAASGLNGCCRCGARGPESRLLEFYLAMPFATPM
EAELARRSLAQDAPPLPVPGVLLKEFTVSGNILTIRLTAADHRQLQLSIS
SCLQQLSLLMWITQCFLPVFLAQPPSGQRR

In some embodiments, an isolated immunostimulatory NY-ESO-1 peptide is provided. In some embodiments, an isolated immunodominant NY-ESO-1 peptide is provided. In some embodiments, an NY-ESO-1 peptide is provided that is a fragment of a NY-ESO-1 protein, for example, human NY-ESO-1 protein. In some embodiments, an isolated MHC class II molecule bound to a NY-ESO-1 peptide, also referred to as a NY-ESO-1 peptide-loaded MHC class II molecule, is provided. In some embodiments, a complex of a NY-ESO-1 peptide-loaded MHC class II molecule with at least one additional MHC class II molecule, each one of the at least one additional MHC class II molecule optionally NY-ESO-1 peptide-loaded, is provided. In some embodiments, such a multimeric NY-ESO-1 peptide-loaded MHC class II complex comprises four NY-ESO-1 peptide loaded MHC class II molecules.

In some embodiments, an isolated protein, polypeptide, polypeptide complex, multimer, and/or tetramer is provided. The term “polypeptide” is used interchangeably with the terms “peptide” and “protein” herein and refers to a polymer molecule comprising at least two amino acids linked by a peptide bond. Amino acids comprised in the polypeptides provided herein may be naturally occurring amino acids or non-naturally occurring amino acids, as well as modified amino acids. Non-naturally occurring and modified amino acids are well known in the art. Polypeptides may also comprise one or more non-peptide bonds.

The term “isolated” as used herein, refers to a molecule or agent, for example a NY-ESO-1 peptide, that has been removed from its natural source, biological environment or milieu (for example by removing a protein from an intact cell source).

A “molecule” may be any chemical or biological molecule, whether naturally occurring or non-naturally occurring/synthetic, for example any molecule that can be made by chemical synthetic methods, recombinant methods or other method known in the art. A molecule can be, for example, a biomolecule, for example a protein, polypeptide or oligopeptide, a nucleic acid, for example an oligonucleotide or polynucleotide, a saccharide, a fatty acid, a sterol, an isoprenoid, a purine, a pyrimidine, a molecule in a complex of molecules, a chemical substance, for example a small organic compound (or molecule), whether naturally occurring or non-naturally occurring or synthetic. A derivative or structural analog of any of the above, or a combination thereof and the like. A small organic compound (molecule) is a chemical compound (or molecule) having a molecular weight of more than 50 yet less than about 2500, preferably less than about 1000 and, more preferably, less than about 500.

In some embodiments, an isolated NY-ESO-1 polypeptide is provided. In some embodiments, an isolated immunostimulatory NY-ESO-1 epitope is provided. The term “epitope”, as used herein, refers to a part of a macromolecule that is recognized by the immune system, for example, a macromolecule that can be specifically bound by, for example, an antibody, a B cell receptor, or a T cell receptor, a macrophage receptor, or a dendritic cell receptor. The epitope is also known as the antigenic determinant. Accordingly, a protein or polypeptide may comprise an epitope but not all proteins or polypeptides comprise an epitope. In some embodiments, a NY-ESO-1 epitope specifically binds to a MHC class II molecule. In some embodiments, a NY-ESO-1 epitope is specifically bound by or can specifically bind to a MHC-DRB1*0101 (MHC-DR1) restricted CD4⁺ T cell. In some embodiments, a NY-ESO-1 epitope is specifically bound by or can specifically bind to a MHC-DRB1*0101 (MHC-DR1) restricted CD4⁺ T cell.

In some embodiments, a NY-ESO-1 peptide-loaded MHC class II molecule is provided. The term “peptide-loaded” signifies a MHC class II molecule with a target peptide epitope bound in its antigen-binding groove. A target epitope is the epitope specifically recognized by the binding groove of the respective MHC class II molecule.

“Specific binding” or “specific recognition” are art recognized terms, used interchangeably herein, and refer to one or more qualities of the binding of two molecules, also referred to herein as binding partners. The specificity of an epitope-binding protein can be determined based on affinity and/or avidity. The affinity, represented by the equilibrium constant for the dissociation of an antigen with an antigen-binding protein, also called the dissociation constant, or K_{D}, is a measure for the binding strength between two binding partners, for example between a NY-ESO-1 epitope and an MHC class II protein, or between a NY-ESO-1 peptide-loaded MHC class II molecule and a T-cell receptor. The lower the value of the K_{D}, the stronger the binding strength between the binding partners. Typically, specific binding or specific recognition between two biomolecules, for example proteins, has a dissociation constant (K_{D}) in the range of 10⁻⁴ to 10⁻¹² moles/liter or less. A K_{D} value greater than 10⁻⁴ mol/liter is generally considered to indicate non-specific binding. A binding affinity of less than 500 nM, less than 200 nM, less than 10 nM, and/or less than 500 μM is preferred. The K_{D} can also be expressed as the ratio of the dissociation rate constant of a complex, denoted as k_{off}, to the its association rate constant, denoted kₒₙ (so that K_{D}=k_{off}/kₒₙ). An on-rate indicative of specific binding between two molecules may vary between 10² M⁻¹s⁻¹ to about 10⁷ M⁻¹s⁻¹. An off-rate indicative of specific binding may vary between 10⁻⁶ s⁻¹ (near irreversible complex with a t_{1/2} of multiple days) to 1 s⁻¹ (t_{1/2}=0.69 s). The affinity of a NY-ESO-1 peptide to an MHC class II molecule, for example a DRB*0202 MHC class II molecule, may vary depending on the length and the sequence of the NY-ESO-1 peptide. Methods to determine the binding affinity or avidity for any given binding between two molecules, for example between two molecules provided herein (e.g., a NY-ESO-1 peptide and a MHC class II molecule, a NY-ESO-1 peptide-loaded MHC class II molecule and a T-cell receptor, or a NY-ESO-1 peptide-loaded MHC class II molecule multimer and a plurality of T-cell receptors) are well known in the art. Methods for predicting binding affinity of a given peptide to a given MHC class II molecule are also well known to those of skill in the art (see, e.g., Chang et al., Peptide length-based prediction of peptide-MHC class II binding, Structural Bioinformatics, 2006; and Salomon et al., Predicting Class II MHC-Peptide binding: a kernel based approach using similarity scores, BMC Bioinformatics, 2006). In some embodiments, specific binding between a NY-ESO-1 peptide and an MHC class II molecule is characterized by a K_{D} value of less than 10⁻⁴ moles/liter. In some embodiments, specific binding between a NY-ESO-1 peptide and an MHC class II molecule is characterized by a K_{D} value of less than 10⁻⁵ moles/liter. In some embodiments, specific binding between a NY-ESO-1 peptide and an MHC class II molecule is characterized by a K_{D} value of less than 10⁻⁶ moles/liter. In some embodiments, specific binding between a NY-ESO-1 peptide and an MHC class II molecule is characterized by a K_{D} value of less than 10⁻⁷ moles/liter. In some embodiments, specific binding between a NY-ESO-1 peptide and an MHC class II molecule is characterized by a K_{D} value of less than 10⁻⁸ moles/liter. In some embodiments, specific binding between a NY-ESO-1 peptide and an MHC class II molecule is characterized by a K_{D} value of less than 10⁻⁹ moles/liter. In some embodiments, specific binding between a NY-ESO-1 peptide and an MHC class II molecule is characterized by a K_{D} value of less than 10⁻¹⁰ moles/liter. In some embodiments, specific binding between a NY-ESO-1 peptide and an MHC class II molecule is characterized by a K_{D} value of less than 10⁻¹¹ moles/liter. In some embodiments, specific binding between a NY-ESO-1 peptide and an MHC class II molecule is characterized by a K_{D} value of less than 10⁻¹² moles/liter.

In some embodiments, a NY-ESO-1 peptide-loaded MHC class II molecule is provided, wherein the NY-ESO-1 peptide comprises at least 9 contiguous amino acid residues of the NY-ESO-1 polypeptide (SEQ ID NO: 1). In some embodiments, a NY-ESO-1 peptide-loaded MHC class II molecule is provided, wherein the NY-ESO-1 peptide comprises 9, 10, 11, 12, 13, 14, 15, 16, 17, 18, 19, 20, 21, 22, 23, 24, 5, 26, 27, 28, 28, 29, 30, 31, 32, 33, 34, 35, 36, 37, 38, 39, or 40 contiguous amino acid residues of the NY-ESO-1 polypeptide (SEQ ID NO: 1). In some embodiments, a NY-ESO-1 peptide-loaded MHC class II molecule is provided, wherein the NY-ESO-1 peptide comprises more than 40 contiguous amino acid residues of the NY-ESO-1 polypeptide (SEQ ID NO: 1). In some embodiments, the NY-ESO-1 peptide of the NY-ESO-1 peptide-loaded MHC class II molecule provided comprises or consists of a NY-ESO-1 peptide of 9-25 contiguous amino acids of SEQ ID NO: 1. In some embodiments, the NY-ESO-1 peptide of the NY-ESO-1 peptide-loaded MHC class II molecule comprises or consists of amino acid residues 123-137 of SEQ ID NO: 1. In some embodiments, the NY-ESO-1 peptide comprises or consists of one of the following amino acid sequences:

(SEQ ID NO: 2)
PGVLLKEFTVSGNILTIRLTAADHR
(SEQ ID NO: 3)
PGVLLKEFTVSGNILTIRLTAADH
(SEQ ID NO: 4)
PGVLLKEFTVSGNILTIRLTAAD
(SEQ ID NO: 5)
PGVLLKEFTVSGNILTIRLTAA
(SEQ ID NO: 6)
PGVLLKEFTVSGNILTIRLTA
(SEQ ID NO: 7)
PGVLLKEFTVSGNILTIRLT
(SEQ ID NO: 8)
PGVLLKEFTVSGNILTIRL
(SEQ ID NO: 9)
PGVLLKEFTVSGNILTIR
(SEQ ID NO: 10)
GVLLKEFTVSGNILTIRLTAADHR
(SEQ ID NO: 11)
GVLLKEFTVSGNILTIRLTAADH
(SEQ ID NO: 12)
GVLLKEFTVSGNILTIRLTAAD
(SEQ ID NO: 13)
GVLLKEFTVSGNILTIRLTAA
(SEQ ID NO: 14)
GVLLKEFTVSGNILTIRLTA
(SEQ ID NO: 15)
GVLLKEFTVSGNILTIRLT
(SEQ ID NO: 16)
GVLLKEFTVSGNILTIRL
(SEQ ID NO: 17)
GVLLKEFTVSGNILTIR
(SEQ ID NO: 18)
VLLKEFTVSGNILTIRLTAADHR
(SEQ ID NO: 19)
VLLKEFTVSGNILTIRLTAADH
(SEQ ID NO: 20)
VLLKEFTVSGNILTIRLTAAD
(SEQ ID NO: 21)
VLLKEFTVSGNILTIRLTAA
(SEQ ID NO: 22)
VLLKEFTVSGNILTIRLTA
(SEQ ID NO: 23)
VLLKEFTVSGNILTIRLT
(SEQ ID NO: 24)
VLLKEFTVSGNILTIRL
(SEQ ID NO: 25)
VLLKEFTVSGNILTIR
(SEQ ID NO: 26)
LLKEFTVSGNILTIRLTAADHR
(SEQ ID NO: 27)
LLKEFTVSGNILTIRLTAADH
(SEQ ID NO: 28)
LLKEFTVSGNILTIRLTAAD
(SEQ ID NO: 29)
LLKEFTVSGNILTIRLTAA
(SEQ ID NO: 30)
LLKEFTVSGNILTIRLTA
(SEQ ID NO: 31)
LLKEFTVSGNILTIRLT
(SEQ ID NO: 32)
LLKEFTVSGNILTIRL
(SEQ ID NO: 33)
LLKEFTVSGNILTIR
(SEQ ID NO: 34)
LKEFTVSGNILTIRLTAADHR
(SEQ ID NO: 35)
LKEFTVSGNILTIRLTAADH
(SEQ ID NO: 36)
LKEFTVSGNILTIRLTAAD
(SEQ ID NO: 37)
LKEFTVSGNILTIRLTAA
(SEQ ID NO: 38)
LKEFTVSGNILTIRLTA
(SEQ ID NO: 39)
LKEFTVSGNILTIRLT
(SEQ ID NO: 40)
LKEFTVSGNILTIRL
(SEQ ID NO: 41)
LKEFTVSGNILTIR
(SEQ ID NO: 42)
KEFTVSGNILTIRLTAADHR
(SEQ ID NO: 43)
KEFTVSGNILTIRLTAADH
(SEQ ID NO: 44)
KEFTVSGNILTIRLTAAD
(SEQ ID NO: 45)
KEFTVSGNILTIRLTAA
(SEQ ID NO: 46)
KEFTVSGNILTIRLTA
(SEQ ID NO: 47)
KEFTVSGNILTIRLT
(SEQ ID NO: 48)
KEFTVSGNILTIRL
(SEQ ID NO: 49)
KEFTVSGNILTIR
(SEQ ID NO: 50)
EFTVSGNILTIRLTAADHR
(SEQ ID NO: 51)
EFTVSGNILTIRLTAADH
(SEQ ID NO: 52)
EFTVSGNILTIRLTAAD
(SEQ ID NO: 53)
EFTVSGNILTIRLTAA
(SEQ ID NO: 54)
EFTVSGNILTIRLTA
(SEQ ID NO: 55)
EFTVSGNILTIRLT
(SEQ ID NO: 56)
EFTVSGNILTIRL
(SEQ ID NO: 57)
EFTVSGNILTIR
(SEQ ID NO: 58)
TVSGNILTIRL
(SEQ ID NO: 59)
TVSGNILTI
(SEQ ID NO: 60)
EFTVSGNILTI

It will be appreciated by those of skill in the art that substitution of an amino acid residue in an amino acid sequence of a polypeptide with a residue of similar structure may result in a polypeptide of similar or even the same function as the original polypeptide. In particular, NY-ESO-1 amino acid residues that are not part of a NY-ESO-1 epitope may be substituted for similar or dissimilar residues and residues that are involved in binding of a NY-ESO-1 epitope by a MHC class II molecule may be substituted with highly similar residues, for example residues that share certain parameters, such as size, charge. Such so-called “conservative” or “functional” amino acid substitutions can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-3 357 768, WO 98/49185, WO 00/46383 and WO 01/09300; and types and/or combinations of such substitutions may be selected on the basis of the pertinent teachings from WO 04/037999 as well as WO 98/49185 and from the further references cited therein. “Conservative” and “functional” substitutions are known to those of skill in the art and are encompassed within the scope of the embodiments described herein.

Conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: H is, Arg and Lys; (d) large aliphatic, nonpolar residues: Met, Leu, Ile, Val and Cys; and (e) aromatic residues: Phe, Tyr and Trp.

Particularly preferred conservative substitutions are as follows: Ala to Gly or to Ser; Arg to Lys; Asn to Gln or to H is; Asp to Glu; Cys to Ser; Gln to Asn; Glu to Asp; Gly to Ala or to Pro; H is to Asn or to Gln; Ile to Leu or to Val; Leu to Ile or to Val; Lys to Arg, to Gln or to Glu; Met to Leu, to Tyr or to Ile; Phe to Met, to Leu or to Tyr; Ser to Thr; Thr to Ser; Trp to Tyr; Tyr to Trp; and/or Phe to Val, to Ile or to Leu.

Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Nat. Acad. Sci. USA 81: 140-144, 1984; Kyte & Doolittle; J. Molec. Biol. 157: 105-132, 1981, and Goldman et al., Ann. Rev. Biophys. Chem. 15: 321-353, 1986, all incorporated herein in their entirety by reference.

In silico methods for designing peptides and proteins equivalent in structure to the NY-ESO-1 peptides provided herein are well known in the art. Methods to predict with high accuracy which amino acid residues in a given peptide or protein may be substituted may include the use of computational strategies for protein engineering, such as combinatorial protein design strategies. Combinatorial protein design strategies allow for the identification of substitutable amino acid residues within a peptide or protein and amino acids for substitution of a given residue by sequence alignment, for example of different peptide or protein amino acid sequences of functionally equivalent proteins found in one species, or across a number of species. As an example, if an alignment of human NY-ESO-1 proteins shows amino acid sequence variability at a certain position of the aligned sequences, the residue at the respective position is indicated to be substitutable with any of the amino acids found at that position within the aligned proteins. Further, any conservative amino acid substitution at that position is highly likely to result in a functional protein or peptide. Similarly, alignment of NY-ESO-1 protein sequences with equivalent or similar structure and/or function from different species can be used to identify substitutable amino acid residues and amino acids for substitution at an identified position.

Methods to determine whether an envisioned sequence modification will result in a functional or non-functional protein or peptide are well known in the art. For example, functional, substituted NY-ESO-1 proteins, protein fragments, or peptides, may be identified by analysing the structure or function of a given modified NY-ESO-1 sequence in silico, for example using methods of in silico prediction of protein structure from a given amino acid sequence, modelling of protein folding, modelling of protein-protein docking, modelling of protein-protein, peptide-protein, or peptide-peptide binding. The in silico results for a modified NY-ESO-1 sequence can be compared to a native NY-ESO-1 sequence, for example, in order to determine whether the modified NY-ESO-1 sequence binds to a MHC-DRB3*0202 (DRb52) protein or a DRB1*0101 protein with similar affinity as compared to the native sequence.

In silico methods suitable for the design of substituted and/or modified NY-ESO-1 sequences with equivalent function to the original sequences, are well known in the art and described in more detail, for example in Lutz and Bornscheuer, Protein Engineering Handbook, Wiley-VCH, (2009), ISBN: 978-3527318506; Mueller and Arndt, Protein Engineering Methods (Methods in Molecular Biology), Humana Press, (1), 2006, ISBN: 978-1588290724; Zaki and Bystroff, Protein Structure Prediction (Methods in Molecular Biology), Humana Press, (2), 2007, ISBN: 978-1588297525; Xu et al., Computational Methods for Protein Structure Prediction and Modeling: Volume 2: Structure Prediction (Biological and Medical Physics, Biomedical Engineering), Springer; (1), 2006, ISBN: 978-0387333212; and Kukol, Molecular Modeling of Proteins (Methods in Molecular Biology), Humana Press, 2008, ISBN: 978-1588298645; all of which are incorporated herein by reference.

In some embodiments, the β-chain of a MHC class II molecule is encoded by the DRB3 gene locus. In some embodiments, the β-chain of a MHC class II molecule is encoded by the DRB3*0202 gene locus. In some embodiments, the β-chain of a MHC class II molecule is a DR52b protein. In some embodiments, the β-chain of a MHC class II molecule is a polypeptide comprising an amino acid sequence sharing more than 70%, more than 80%, more than 90%, more than 95%, more than 98%, more than 99%, or more than 99.9% sequence identity with the amino acid sequence of a human DR52b protein. In some embodiments, the MHC class II molecule is NY-ESO-1 peptide-loaded.

In some embodiments, the β-chain of a MHC class II molecule is encoded by the DRB1 gene locus. In some embodiments, the β-chain of a MHC class II molecule is encoded by the DRB1*0101 gene locus. In some embodiments, the β-chain of a MHC class II molecule is a DR1 protein. In some embodiments, the β-chain of a MHC class II molecule is a polypeptide comprising an amino acid sequence sharing more than 70%, more than 80%, more than 90%, more than 95%, more than 98%, more than 99%, or more than 99.9% sequence identity with the amino acid sequence of a human DR1 protein. In some embodiments, the MHC class II molecule is NY-ESO-1 peptide-loaded.

In some embodiments, a MHC class II molecule is provided that is linked to a ligand. In some embodiments, the ligand is a ligand of a binding molecule, for example a monovalent binding molecule or a multivalent binding molecule. In some embodiments, the MHC class II molecule is covalently linked to the ligand, for example, by peptide bond. The term “covalent bond” is art-recognized and refers to a bond between two atoms where electrons are attracted electrostatically to both nuclei of the two atoms, and the net effect of increased electron density between the nuclei counterbalances the internuclear repulsion. The term “covalent bond” further includes coordinate bonds when the bond is with a metal ion. In some embodiments, the ligand is a peptide. In some embodiments, the ligand and one of the polypeptide chains, for example the α-chain or the β-chain, of the MHC class II molecule are encoded by the same nucleic acid sequence. In some embodiments, the ligand is biotin or a functional equivalent and the multivalent binding molecule is streptavidin or avidin or a functional equivalent of any of these. In some embodiments, a biotinylated MHC class II molecule is provided, wherein the MHC molecule and the biotin residue are linked, for example, via primary amine biotinylation, sulfhydryl biotinylation, carboxyl biotinylation, glycoprotein biotinylation, or non-specific biotinylation. In some embodiments, the binding molecule is a soluble molecule. In some embodiments, the binding molecule is attached to a solid support, for example by covalent bond or by non-covalent bond or interaction. Other mono-, bi-, tri-, tetra-, and multivalent binding molecules are well known in the art.

In some embodiments, MHC class II multimers are provided. In some embodiments, such MHC class II multimers comprise a multivalent binding molecule binding a plurality of MHC class II molecules linked to a ligand of the multivalent binding molecule. At least one of the MHC class II molecules in a NY-ESO-1 peptide-loaded MHC class II multimer, as provided by some embodiments, is loaded with a NY-ESO-1 peptide. In some embodiments, all MHC class II molecules in a MHC class II multimer are peptide-loaded. For example, a NY-ESO-1 peptide-loaded MHC class II tetramer comprises four MHC class II molecules, of which one, two three, or all four may be NY-ESO-1 peptide-loaded. In some embodiments, a multivalent binding molecule, for example streptavidin or avidin, binds to a plurality of ligand-linked, and optionally NY-ESO-1 peptide-loaded MHC class II molecules as provided herein such that a multimer is formed. In some embodiments, the MHC class II multimer is a MHC class II tetramer.

Some embodiments provide methods for producing a peptide-loaded MHC molecule multimer, for example an NY-ESO-1 peptide-loaded MHC class II tetramer. In some embodiments, an MHC molecule is contacted with an MHC molecule-binding peptide. MHC molecule-binding peptides are well known in the art. Further examples of MHC molecule-binding peptides include the MHC molecule binding NY-ESO-1 peptides provided herein and the peptides disclosed in Table 2. In some embodiments, the MHC molecule is a MHC class II molecule. In some embodiments, the MHC molecule is linked to a ligand. In some embodiments, the MHC molecule is linked to a ligand prior to being contacted with the MHC molecule-binding peptide. In some embodiments, the MHC molecule is linked to a ligand after being contacted with the MHC molecule-binding peptide.

In some embodiments, the MHC molecule is contacted with a MHC molecule-binding peptide that is conjugated to a tag. In some embodiments, the tag is a peptide tag. A protein or peptide may be fused to a peptide tag by methods well known to those in the art. For example, expression vectors allowing for the in-frame fusion of a peptide of interest, for example, an MHC molecule-binding peptide, and a peptide tag encoded by the vector are known to skilled persons and are commercially available. For another example, a nucleic acid encoding a peptide tag fused to a peptide or protein may be generated by PCR-amplification of a coding region of the peptide or protein of interest with a primer comprising a nucleic acid sequence encoding the peptide tag. In some embodiments, a nucleic acid encoding a peptide or protein fused to a peptide tag may be synthesized de novo. In some embodiments, a peptide or protein, for example, an MHC molecule-binding peptide, may be fused directly to a peptide tag. In some embodiments, a spacer or linker, for example, a peptide spacer or linker, may connect the peptide and the peptide tag. In some embodiment, a tag is fused to the N-terminus of the peptide. In some embodiments, a tag is fused to the C-terminus of the peptide. In some embodiments, both the C— and the N-terminus of the peptide are tagged, for example, with the same tag on each terminus, or with different tags.

Peptide tags are well known to those of skill in the art and examples of peptide tags include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Sequences of peptide tags useful in some embodiments of this invention, for example, the tags described herein, are well known to those of skill in the art and exemplary sequences and references describing such sequences, as summarized, for example, in Kimple, M. E., and Sondek, J. Overview of affinity tags for protein purification. Curr Protoc Protein Sci. 2004 September; Chapter 9:Unit 9.9, incorporated in its entirety herein for disclosure of peptide tags, are listed in Table 1:

TABLE 1
exemplary peptide tags.

| | | | SEQ ID | |
|---|---|---|---|---|
| Tag | Length | Exemplary sequence(s) | NO: | References |
| Albumin-binding | 137 | | | Nilsson, Bosnes |
| protein (ABP) | | | | et al. 1997 |
| Alkaline | 444 | | | Lazzaroni et al. |
| phosphatase (AP) | | | | 1985 |
| AU1 | 6 | DTYRYI | 85 | Goldstein et al. |
| | | | | 1992 |
| AU5 | 6 | TDFYLK | 86 | Crespo et al. |
| | | | | 1997 |
| Bacteriophage T7 | 11 | MASMTGGQQMG | 87 | Makrides 1996 |
| epitope (T7-tag) | | | | |
| Bacteriophage V5 | 14 | GKPIPNPLLGLDST | 88 | McLean et al. |
| epitope (V5-tag) | | | | 2001 |
| Biotin Carboxyl | 100 | | | Nilsson, Stahl et |
| Carrier Protein | | | | al. 1997 |
| (BCCP) | | | | |
| B-tag | 6 | QYPALT | 89 | Wang et al. 1996 |
| Calmodulin | 26 | | | Terpe 2003 |
| binding peptide | | | | |
| (CBP) | | | | |
| Cellulose binding | 27-189 | | | Terpe 2003 |
| domain (CBD) | | | | |
| Chitin binding | 51 | | | Terpe 2003 |
| domain (CBD) | | | | |
| Chloramphenicol | 218 | | | Podbielski et al. |
| acetyl transferase | | | | 1992 |
| (CAT) | | | | |
| Choline-binding | 145 | | | Jones et al. 1995 |
| domain (CBD) | | | | |
| dihydrofolate | 227 | | | Morandi et al. |
| reductase (DHFR) | | | | 1984 |
| E2-tag | 10 | SSTSSDFRDR | 90 | Kaldalu et al. |
| | | | | 2000 |
| FLAG | 8 | DYKDDDK | 91 | Terpe 2003 |
| Galactose-binding | 509 | | | Jones et al. 1995 |
| protein (GBP) | | | | |
| Glutathione S- | 211 | | | Smith 2000 |
| transferase (GST) | | | | |
| Green Fluorescent | 220 | | | Gerdes et al. |
| protein (GFP) | | | | 1996 |
| Hemagglutinin | 31 | | | Tai et al. 1988 |
| (HA) | | | | |
| Histidine-affinity | 19 | KDHLIHNVHKEFHAHAH | 95 | Terpe 2003 |
| tag (HAT) | | NK | | |
| Ketosteroid | 125 | | | Kuliopulos et al. |
| isomerase (KSI) | | | | 1994 |
| KT3 | 11 | KPPTPPPEPET | 96 | Kwatra et al. |
| | | | | 1995 |
| LacZ | 1024 | | | Tai et al. 1988 |
| Luciferase | 551 | | | Karp et al. 1999 |
| Maltose-binding | 396 | | | Nilsson, Stahl et |
| protein (MBP) | | | | al. 1997; Terpe |
| | | | | 2003 |
| Myc | 11 | CEQKLISEEDL | 97 | Kolodziej et al. |
| | | | | 1991 |
| NorpA | 5 | TEFCA | 98 | Kimple et al. |
| | | | | 2002 |
| NusA | 495 | | | Terpe 2003 |
| Polyarginine | 5-6 | RRRRR | 99 | Terpe 2003 |
| (Arg-tag) | | | | |
| Polycysteine | 4 | CCCC | 101 | Stevens 2000 |
| (Cys-tag) | | | | |
| Polyhistidine | 2-12 | HHH, | 102 | Bornhorst et al. |
| (His-tag) | | HHHH, | 103 | 2000 |
| | | HHHHH, | 104 | |
| | | HHHHHH, | 105 | |
| | | HHHHHHH, | 106 | |
| | | HHHHHHHH, | 107 | |
| | | HHHHHHHHH, | 108 | |
| | | HHHHHHHHHH, | 109 | |
| | | HHHHHHHHHHH, | 110 | |
| | | HHHHHHHHHHHH | 111 | |
| Polyphenyalanine | 11 | FFFFFFFFFFF | 112 | Stevens 2000 |
| (Phe-tag) | | | | |
| Protein C | 12 | | | Fritze et al. 2000 |
| S1-tag | 9 | NANNPDWDF | 113 | Berlot 1999 |
| S-tag | 15 | KETAAAKFERQHMDS | 114 | Fritze et al. 2000 |
| Staphylococcal | 280 | | | Nilsson, Stahl et |
| protein A (Protein | | | | al. 1997 |
| A) | | | | |
| Staphylococcal | 280 | | | Nilsson, Stahl et |
| protein G (Protein | | | | al. 1997 |
| G) | | | | |
| Strep-tag | 8-9 | WSHPQFEK, | 115 | Skerra et al. 2000 |
| | | AWAHPQPGG | 116 | |
| Streptavidin | 159 | | | Sano et al. 1998 |
| Streptavidin | 38 | | | Terpe 2003 |
| binding peptide | | | | |
| (SBP) | | | | |
| T7 gene 10 (T7- | 260 | | | Stevens 2000 |
| tag) | | | | |
| Thioredoxin (Trx) | 109 | | | Terpe 2003 |
| trpE | 25-336 | | | Stevens 2000 |
| Ubiquitin | 76 | | | Stevens 2000 |
| Universal | 6 | HTTPHH | 117 | Nelson et al. |
| | | | | 1999 |
| Vesicular | 11 | YTDIEMNRLGK | 118 | Fritze et al. 2000 |
| Stomatitis Virus | | | | |
| Glycoprotein | | | | |
| peptide (VSV-G) | | | | |

### Table 1 References Disclosing Exemplary Peptide Tags and Methods of Use:

Berlot, C. H. 1999. Expression and functional analysis of G-protein alpha subunits in mammalian cells. In G-proteins: Techniques of Analysis, D. R. Manning). pp. 37-57. CRC Press, New York.
Bornhorst, J. A. and J. J. Falke 2000. Purification of proteins using polyhistidine affinity tags. Methods Enzymol 326: 245-54.
Chong, S., F. B. Mersha, et al. 1997. Single-column purification of free recombinant proteins using a self-cleavable affinity tag derived from a protein splicing element. Gene 192: 271-81.
Crespo, P., K. E. Schuebel, et al. 1997. Phosphotyrosine-dependent activation of Rac-1 GDP/GTP exchange by the vav proto-oncogene product. Nature 385: 169-72.
Cronan, J. E., Jr. 1990. Biotination of proteins in vivo. A post-translational modification to label, purify, and study proteins. J Biol Chem 265: 10327-33.
di Guan, C., P. Li, et al. 1988. Vectors that facilitate the expression and purification of foreign peptides in Escherichia coli by fusion to maltose-binding protein. Gene 67: 21-30.
Fritze, C. E. and T. R. Anderson 2000. Epitope tagging: general method for tracking recombinant proteins. Methods Enzymol 327: 3-16.
Gerdes, H. H. and C. Kaether 1996. Green fluorescent protein: applications in cell biology. FEBS Lett 389: 44-7.
Goldstein, D. J., R. Toyama, et al. 1992. The BPV-1 E5 oncoprotein expressed in Schizosaccharomyces pombe exhibits normal biochemical properties and binds to the endogenous 16-kDa component of the vacuolar proton-ATPase. Virology 190: 889-93.
Jones, C., A. Patel, et al. 1995. Current trends in molecular recognition and bioseparation. J Chromatogr A 707: 3-22.
Kaldalu, N., D. Lepik, et al. 2000. Monitoring and purification of proteins using bovine papillomavirus E2 epitope tags. Biotechniques 28: 456-60, 462.
Karp, M. and C. Oker-Blom 1999. A streptavidin-luciferase fusion protein: comparisons and applications. Biomol Eng 16: 101-4.
Kimple, M. E. and J. Sondek 2002. Affinity tag for protein purification and detection based on the disulfide-linked complex of InaD and NorpA. Biotechniques 33: 578, 580, 584-8 passim.
Kolodziej, P. A. and R. A. Young 1991. Epitope tagging and protein surveillance. Methods Enzymol 194: 508-19.
Kuliopulos, A. and C. T. Walsh 1994. Production, purification, and cleavage of tandem repeats of recombinant peptides. J Am Chem Soc 116: 4599-4607.
Kwatra, M. M., J. Schreurs, et al. 1995. Immunoaffinity purification of epitope-tagged human beta 2-adrenergic receptor to homogeneity. Protein Expr Purif 6: 717-21.
Lazzaroni, J. C., D. Atlan, et al. 1985. Excretion of alkaline phosphatase by Escherichia coli K-12 pho constitutive mutants transformed with plasmids carrying the alkaline phosphatase structural gene. J Bacteriol 164: 1376-80.
Lilius, G., M. Persson, et al. 1991. Metal affinity precipitation of proteins carrying genetically attached polyhistidine affinity tails. Eur J Biochem 198: 499-504.
Ljungquist, C., A. Breitholtz, et al. 1989. Immobilization and affinity purification of recombinant proteins using histidine peptide fusions. Eur J Biochem 186: 563-9.
Maina, C. V., P. D. Riggs, et al. 1988. An Escherichia coli vector to express and purify foreign proteins by fusion to and separation from maltose-binding protein. Gene 74: 365-73.
Makrides, S. C. 1996. Strategies for achieving high-level expression of genes in Escherichia coli. Microbiol Rev 60: 512-38.
McLean, P. J., H. Kawamata, et al. 2001. Alpha-synuclein-enhanced green fluorescent protein fusion proteins form proteasome sensitive inclusions in primary neurons. Neuroscience 104: 901-12.
Morandi, C., M. Perego, et al. 1984. Expression of human dihydrofolate reductase cDNA and its induction by chloramphenicol in Bacillus subtilis. Gene 30: 69-77.
Nelson, R. W., J. W. Jarvik, et al. 1999. BIA/MS of epitope-tagged peptides directly from E. coli lysate: multiplex detection and protein identification at low-femtomole to subfemtomole levels. Anal Chem 71: 2858-65.
Nilsson, J., M. Bosnes, et al. 1997. Heat-mediated activation of affinity-immobilized Taq DNA polymerase. Biotechniques 22: 744-51.
Nilsson, J., S. Stahl, et al. 1997. Affinity fusion strategies for detection, purification, and immobilization of recombinant proteins. Protein Expr Purif 11: 1-16.
Podbielski, A., J. A. Peterson, et al. 1992. Surface protein-CAT reporter fusions demonstrate differential gene expression in the vir regulon of Streptococcus pyogenes. Mol Microbiol 6: 2253-65.
Sano, T., S. Vajda, et al. 1998. Genetic engineering of streptavidin, a versatile affinity tag. J Chromatogr B Biomed Sci Appl 715: 85-91.
Skerra, A. and T. G. Schmidt 2000. Use of the Strep-Tag and streptavidin for detection and purification of recombinant proteins. Methods Enzymol 326: 271-304.
Smith, D. B. 2000. Generating fusions to glutathione S-transferase for protein studies. Methods Enzymol 326: 254-70.
Smith, D. B. and K. S. Johnson 1988. Single-step purification of polypeptides expressed in Escherichia coli as fusions with glutathione S-transferase. Gene 67: 31-40.
Smith, M. C., T. C. Furman, et al. 1988. Chelating peptide-immobilized metal ion affinity chromatography. A new concept in affinity chromatography for recombinant proteins. Biol Chem 263: 7211-5.
Stevens, R. C. 2000. Design of high-throughput methods of protein production for structural biology. Structure Fold Des 8: R177-85.
Tai, T. N., W. A. Havelka, et al. 1988. A broad-host-range vector system for cloning and translational lacZ fusion analysis. Plasmid 19: 175-88.
Terpe, K. 2003. Overview of tag protein fusions: from molecular and biochemical fundamentals to commercial systems. Appl Microbiol Biotechnol 60: 523-33.
Wang, L. F., M. Yu, et al. 1996. BTag: a novel six-residue epitope tag for surveillance and purification of recombinant proteins. Gene 169: 53-8.

The table 1 references listed above are incorporated in their entirety herein for disclosure of peptide tags and methods of using such tags.

For further references providing an overview of useful peptide tags and methods for the purification of tagged peptides or proteins, see, e.g., Weiss E, Chatellier J, Orfanoudakis G., In vivo biotinylated recombinant antibodies: construction, characterization, and application of a bifunctional Fab-BCCP fusion protein produced in Escherichia coli. Protein Expr Purif. 1994 October; 5(5):509-17; Funakoshi M, Hochstrasser M., Small epitope-linker modules for PCR-based C-terminal tagging in Saccharomyces cerevisiae. Yeast. 2009 March; 26(3):185-92; Moqtaderi Z, Struhl K., Expanding the repertoire of plasmids for PCR-mediated epitope tagging in yeast. Yeast. 2008 April; 25(4):287-92; Tagwerker C, Zhang H, Wang X, Larsen L S, Lathrop R H, Hatfield G W, Auer B, Huang L, Kaiser P., HB tag modules for PCR-based gene tagging and tandem affinity purification in Saccharomyces cerevisiae. Yeast. 2006 June; 23(8):623-32; Kobayashi T, Morone N, Kashiyama T, Oyamada H, Kurebayashi N, Murayama T.; Engineering a novel multifunctional green fluorescent protein tag for a wide variety of protein research. PLoS One. 2008; 3(12):e3822. Epub 2008 Dec. 2; Lichty J J, Malecki J L, Agnew H D, Michelson-Horowitz D J, Tan S., Comparison of affinity tags for protein purification. Protein Expr Purif. 2005 May; 41(1):98-105.; 18.1. Nag B, Mukku P V, Arimilli S, Kendrick T, Deshpande S V, Sharma S, Separation of complexes of major histocompatibility class II molecules and known antigenic peptide by metal chelate affinity chromatography, J Immunol Methods 1994; 169:273-285; Cabanne C, Pezzini J, Joucla G, Hocquellet A, Barbot C, Garbay B, Santarelli X. Efficient purification of recombinant proteins fused to maltose-binding protein by mixed-mode chromatography. J Chromatogr A. 2009 May 15; 1216(20):4451-6. Epub 2009 Mar. 20. All listed references are incorporated in their entirety herein for disclosure of peptide and protein tags and methods for purification of tagged proteins.

In some embodiments, the MHC molecule-binding peptide is tagged with a histidine (His) tag. A histidine tag, a tag well known to those of skill in the art, comprises a sequence of contiguous histidine residues and can be fused to the N- or the C-terminus of a peptide or protein or inserted into the protein sequence. In some embodiments, the His tag is fused to the N-terminus of the MHC molecule-binding peptide, for example, an MHC class II binding NY-ESO-1 peptide as provided herein. In some embodiments, the His tag is fused to the C-terminus of the MHC molecule-binding peptide, for example, an MHC class II binding NY-ESO-0.1 peptide as provided herein. In some embodiments, the His tag comprises 3, 4, 5, 6, 7, 8, 9, 10, 11, or 12 contiguous histidine residues. In some embodiments, the His tag consists of 6 contiguous histidine residues. In some embodiments, a MHC class II molecule is contacted with a MHC class II binding NY-ESO-1 peptide, as provided herein, that is tagged with a His tag.

In some embodiments, contacting of a MHC molecule with a tagged MHC molecule-binding peptide results in the formation of a complex comprising the MHC molecule bound to the tagged MHC molecule-binding peptide. In some embodiments, a complex comprising the MHC molecule bound to the tagged MHC molecule-binding peptide is isolated using a method suitable for the purification of peptides or proteins carrying the respective tag. Methods for the purification of tagged proteins are well known to those in the art (see, e.g., references cited in relation to protein tags) and the method used in a given embodiment will depend on the nature of the tag employed. In some embodiments, a complex comprising the MHC molecule bound to the tagged MHC molecule-binding peptide is isolated by affinity chromatography. In some embodiments, a ligand specifically binding the tag with high affinity is immobilized on a solid support, for example, a resin or membrane, and contacted with a complex comprising the MHC molecule bound to the tagged MHC molecule-binding peptide. In some embodiments, complex bound by the ligand is separated from non-bound molecules, washed, and subsequently eluted from the ligand. In some embodiments, a complex comprising an MHC class II molecule bound to a His-tagged NY-ESO-1 peptide as provided herein is isolated using affinity chromatography employing an Ni²⁺ resin (see, e.g., Crowe J, Masone B S, Ribbe J. One-step purification of recombinant proteins with the 6×His tag and Ni-NTA resin. Mol. Biotechnol. 1995 December; 4(3):247-58; and The QIAexpressionist™, A handbook for high-level expression and purification of 6×His-tagged proteins, Fifth Edition, June 2003, published by QIAGEN, Inc. (www.qiagen.com), both of which are incorporated herein by reference for disclosure of His-tag peptide expression and isolation/purification, for example, by affinity chromatography).

In some embodiments, a complex comprising the MHC molecule bound to the tagged MHC molecule-binding peptide is isolated by size fractionation. Methods for size fractionation of proteins, peptides, and complexes comprising such molecules are well known in the art and examples of such methods include, but are not limited to size exclusion chromatography (e.g., gel filtration), gradient centrifugation, and dialysis. In some embodiments, a complex comprising the MHC molecule bound to the tagged MHC molecule-binding peptide is isolated by collecting a specific fraction comprising molecules of a specific size and/or molecular weight. In some embodiments, a complex bound by the ligand is isolated in a procedure comprising a step employing affinity chromatography of the peptide tag and a step of size fractionation. In some embodiments, a complex comprising an MHC class II molecule bound to a His-tagged NY-ESO-1 peptide as provided herein is isolated using affinity chromatography employing an Ni²⁺ resin and subsequently using a gel filtration procedure. In some embodiments, the gel filtration procedure employs a resin that has a separation range (Mr) of about 10000 to about 600000. In some embodiments, the resin employed in the gel filtration chromatography is a S200 resin. Methods and resins for gel filtration an size exclusion chromatography are well known to those of skill in the art.

Other methods of producing peptide-loaded MHC multimers are known in the art (for example, see Altman et al., Science 274:94 96, 1996; Dunbar et al., Curr. Biol. 8:413 416, 1998; Crawford et al., Immunity 8:675 682, 1998).

In all embodiments, non-denaturing conditions are preferred during isolation of empty and peptide-loaded MHC class II molecules.

Empty MHC class II molecules generated and/or isolated by methods provided herein and/or using reagents described herein can subsequently be loaded with MHC class II binding peptides. Suitable MHC class II binding peptides for specific MHC class II molecules are well known in the art. Exemplary peptides are described in Table 2 and it will be appreciated that the invention is not limited in this respect.

It will further be appreciated by those of skill in the art that the methods and reagents useful for the preparation of MHC molecules, either “empty” or peptide-loaded, are universally applicable to MHC molecules and MHC molecule-binding antigenic peptides. Specific MHC molecule/peptide pairs are well known to those of skill in the art and have been described, for example in Guillaume P, Dojcinovic D, Luescher I F, Soluble MHC-peptide complexes: tools for the monitoring of T cell responses in clinical trials and basic research. Cancer Immun. 2009 Sep. 25; 9:7.PMID: 19777993; incorporated herein in its entirety for disclosure of the Ludwig Institute for Cancer Research tetramer collection, MHC multimer staining methods and reagents. Further, collections of MHC multimer staining reagents have been generated that disclose suitable MHC/peptide combinations, for example, the Ludwig Institute for Cancer Research tetramer collection, accessible at www.cancerimmunity.org/tetramers/index.htm, the entire contents of which are incorporated herein by reference.

Exemplary MHC/peptide pairs include, but are not limited to

TABLE 2
Exemplary MHC/peptide pairs.

| MHC | Protein | Position | Peptide |
|---|---|---|---|
| HLA- | adenovirus hexon | 911-925 | DEPTLLYVLFEVFDV |
| DP*0401 | CD74/HLA-DR | 103-117 | PVSKMRMATPLLMQA |
| | invariant γ-chain | | |
| | MAGE-3 | 111-125 | RKVAELVHFLLLKYR |
| | | 243-258 | KKLLTQHFVQENYLEY |
| | | 157-170 | SLLMWITQCFLPVF |
| | NY-ESO-1 | 157-18 | SLLMWITQCFLPVFLAQPPSGQRR |
| | tetanus toxin | 947-960 | FNNFTVSFWLRVPK |
| HLA- | influenza HA | 57-76 | QILDGENCTLIDALLGDPQD |
| DQ*0601 | gp100/Pmel 17 | 175-189 | GRAMLGTHTMEVTVY |
| | MELAN-A/MART-1 | 25-36 | EEAAGIGILTVI |
| | | 26-35 | EAAGIGILTV |
| HLA- | CD74/HLA-DR | 103-117 | PVSKMRMATPLLMQA |
| DR*0101 | invariant γ-chain | | |
| | influenza HA | 306-318 | PKYVKQNTLKLAT |
| | MAGE-3 | 267-282 | ACYEFLWGPRALVETS |
| | NY-ESO-1 | 87-98 | LLEFYLAMPFAT |
| | | 123-137 | LKEFTVSGNILTIRL |
| HLA- | CD74/HLA-DR | 103-117 | PVSKMRMATPLLMQA |
| DR*0401 | invariant γ-chain | | |
| | gp100/Pmel 17 | 44-59 | WNRQLYPEWTEAQRLD |
| | influenza M1 | 61-72 | GFVFTLTVPSER |
| | influenza NP | 206-229 | FWRGENGRKTRIAYERMCNILKGK |
| | NY-ESO-1 | 119-143 | PGVLLKEFTVSGNILTIRLTAADHR |
| H-2IAb | chicken ovalbumin | 323-339 | ISQAVHAAHAEINEAGR |
| | mouse DCT/TRP-2 | 110-124 | KFGWSGPDCNRKKPA |
| | LCMV Pre-GP-C | 61-80 | GLNGPDIYKGVYQFKSVEFD |
| | mouse TRP-1 | 420-434 | ADIYTFPLENAPIGH |

In some embodiments, soluble MHC molecules are produced, for example, according to methods provided herein or known in the art. In some embodiments, a peptide which binds the MHC molecule, for example an immunodominant NY-ESO-1 epitope comprising peptide, is contacted with a soluble MHC molecule under conditions suitable for the MHC molecule to bind the peptide. In some embodiments, the isolated MHC/peptide complex is then linked to a ligand of a binding molecule. In some embodiments, the ligand is biotin. In some embodiments, peptide-loaded and ligand-linked MHC molecules are then contacted with a binding molecule. In some embodiments, the binding molecule is a multivalent binding molecule that binds a plurality of ligand-linked MHC molecules. In some embodiments, the multivalent binding molecule is streptavidin or avidin. In some embodiments, the multivalent binding molecule is labeled with a detectable label, for example, phycoerythrin. In some embodiments, the multivalent binding molecule binds four MHC molecules, resulting in the formation of a tetrameric MHC molecule complex, also referred to herein as a MHC tetramer. In some embodiments, the MHC molecule is a MHC class II molecule. In some embodiments, the MHC class II molecule is a DRB3*0202 encoded DR52b molecule. In some embodiments, the MHC class II molecule is a DRB1*0101 encoded DR1 molecule. In some embodiments, all MHC molecules of a tetramer are loaded with a NY-ESO-1 peptide. In some embodiments, the multimeric or tetrameric MHC complex is labeled with a detectable label, for example a fluorophore, a radioactive label, an enzyme, a tag, a binding molecule, an antibody or antigen-binding fragment thereof.

In some embodiments, a multimeric MHC molecule complex, comprising at least one NY-ESO-1 peptide-loaded MHC class II molecule, is contacted with a cell of the immune system or a population of cells comprising such a cell, for example, blood cells or cells obtained from a lymph node, under conditions suitable for the peptide-loaded MHC class II molecules to bind a T-cell receptor on the surface of a T-cell receptor expressing cell, for example a DRB3*0202 restricted CD4⁺ T-cell or a DRB1*0101 restricted CD4⁺ T-cell in a population of cells. Suitable conditions are well known to those of skill in the art. In some embodiments, suitable conditions are physiological conditions. Buffers and reagents to generate suitable, for example physiological conditions, are well known in the art. Cells bound by the multimeric MHC complex can be identified and isolated by various methods known in the art, for example flow cytometry, fluorescence activated cell sorting (FACS), fluorescence microscopy, and others. The isolated cells can then be expanded in vitro for uses as described herein. In some embodiments, a cell bound by multimeric NY-ESO-1 peptide-loaded MHC class II molecule complexes is expanded as an isolated, clonal cell population. Accordingly, in some embodiments, a method for the isolation and clonal expansion of DRB3*0202 restricted CD4⁺ T-cells or DRB1*0101 restricted CD4⁺ T-cell recognizing NY-ESO-1 peptide-loaded MHC class II molecules is provided. In some embodiments, a population of DRB3*0202 restricted CD4⁺ T-cells or of a DRB1*0101 restricted CD4⁺ T-cell recognizing NY-ESO-1 is administered to a recipient after isolation from a donor and, optionally, expansion in vitro. In some embodiments, the donor and the recipient are the same subject. In some embodiments, the donor and the recipient are different subjects, for example, at least partially genetically matched subjects.

In some embodiments, multimeric NY-ESO-1 peptide-loaded MHC class II tetramers are used to monitor DRB3*0202 restricted CD4⁺ T-cell or DRB1*0101 restricted CD4⁺ T-cell responses to vaccination protocols, for example to administration of an immunostimulatory NY-ESO-1 peptide or a composition comprising such a peptide. In some embodiments, a cell population is obtained from a subject suspected or diagnosed to have a tumor. In some embodiments, the cell population is then contacted with a NY-ESO-1 peptide-loaded MHC class II molecule or multimer and the binding of the NY-ESO-1 peptide-loaded MHC class II molecule or multimer to a DRB3*0202 restricted CD4⁺ T cell or a DRB1*0101 restricted CD4⁺ T cell recognizing NY-ESO-1 peptide-loaded MHC-class II molecules is detected. In some embodiments, the results from the detection are then used to determine the presence, quantity, and/or frequency of DRB3*0202 restricted CD4⁺ T cells or of DRB1*0101 restricted CD4⁺ T cells recognizing NY-ESO-1 peptide-loaded MHC-class II molecules in the subject. This procedure may be performed before, during and/or after administration of an immunostimulatory NY-ESO-1 peptide, fragment, and/or vaccine to the subject. In some embodiments, monitoring of a DRB3*0202 or DRB1*0101 restricted CD4⁺ T cell population recognizing NY-ESO-1 peptide-loaded MHC-class II molecules in a subject diagnosed with or having a tumor is used to analyze an immune response to an immunostimulatory NY-ESO-1 peptide in the subject, as detailed below. In some embodiments, the results from methods determining whether DRB3*0202 or DRB1*0101 restricted CD4⁺ T cells recognizing NY-ESO-1 peptide-loaded MHC-class II molecules are present in a subject having a tumor are used to classify the tumor, to stage the disease, to determine a prognosis of disease development, and/or to chose a treatment, for example a vaccination approach, a chemotherapeutic approach, and/or a radiation therapy approach.

Methods to isolate a suitable cell source or cell population from a subject are known in the art. In some embodiments, a cell population is obtained from a subject. In some embodiments, the cell population is a blood cell population, for example, a peripheral blood cell population. In some embodiments, a subpopulation of cells, for example a peripheral blood mononuclear cell (PBMCs) population, a B-cell population, or a T-cell population, is isolated from a peripheral blood sample from a subject by methods known in the art, for example by selective lysis, FACS or MACS for specific antigens, such as CD4 or CD8, etc. In some embodiments, cells are isolated from a lymph node, for example from a lymph node biopsy.

In some embodiments, the detection and quantification methods described herein may be used to determine an amount of a therapeutic agent or composition, for example, an amount of an immunostimulatory NY-ESO-1 peptide, sufficient to induce or enhance an immune response in a subject. In some embodiments, the amount of an administered agent, for example the amount of an immunostimulatory NY-ESO-1 peptide, may be adjusted based on the results of the detection and quantification methods described herein. For example, the amount of an administered agent may be increased, for example by additional administration (e.g. of the same dose, a higher dose or a lower dose) or by repeated administration, until a desired effect on the quantity, frequency, and/or proliferation rate of a certain cell population is detected. Alternatively, the amount of an administered agent may be decreased to the lowest amount at which a desired effect on the quantity, frequency, and/or proliferation rate of a certain cell population is detected.

An immune response induced or enhanced in a subject, for example, by administration of an immunogenic NY-ESO-1 epitope comprising peptide, can be monitored by various methods known in the art. For example, the presence of T cells specific for a given antigen or epitope can be detected by direct labeling of T cell receptors with labeled MHC molecules or multimers, which present the antigenic peptide. NY-ESO-1 peptide-loaded MHC class II multimers, for example tetramers, bind specific T-cell receptors with appropriate specificity and affinity, allowing for the specific labeling of certain subtypes of T-cells, for example DRB3*0202-restricted or DRB1*0101-restricted CD4+ T-cells that can bind NY-ESO-1 peptide-loaded MHC class II molecules containing a DR52b polypeptide or a DR1 polypeptide, respectively.

In some embodiments, a polypeptide and/or polypeptide complex provided herein, for example a NY-ESO-1 epitope, a MHC class II molecule, a MHC class II molecule complex, etc., is labeled with or coupled to or conjugated with a detectable label or detectable group. In some embodiments, a particular label is chosen that does not significantly interfere with the specific binding of the polypeptide or MHC class II multimer or other molecule used in any of the methods provided herein. A detectable label or group can be any material having a detectable physical or chemical property. Such detectable labels are well known in the art. The particular label chosen in any given embodiment will, of course, depend on the assay or method of detection to be employed. In general, almost any label useful in known assays or methods of detection can be applied to the methods of the present invention. Thus, a label is any composition detectable by spectroscopic, photochemical, biochemical, immunochemical, electrical, optical, radiological or chemical means. Useful labels in the present invention include but are not limited to magnetic beads (e.g. Dynabeads™), fluorescent dyes (e.g. fluorescein isothiocyanate, Texas red, rhodamine, Cy3, Cy5, Cy5.5, Alexa 647 and derivatives), radiolabels (e.g. ³H, ¹¹²H, ³⁵S, ¹⁴C, ³²P or ^{99m}Tc), enzymes (e.g. horseradish peroxidase, alkaline phosphatase and others commonly used in an ELISA), tags, binding molecules, for example antibodies or antigen-binding fragments thereof, and colorimetric labels such as colloidal gold, colored glass or plastic (e.g. polystyrene, polypropylene, latex, etc.) beads.

As used herein, the terms “labeled with” and “conjugated with” are intended to refer to, but not to be limited to, two or more molecules, bound to each other by one or more of the following: one or more covalent bonds, one or more ionic-bonds, one or more permanent dipole bonds, one or more instantaneous dipole to induced dipole bonds (e.g., van der Waals). The label may be coupled directly or indirectly to a polypeptide, for example a MHC class II polypeptide, or any other molecule, for example a binding molecule, provided by the invention according to methods well known in the art.

As indicated above, a wide variety of labels may be used, with the choice of label depending on the sensitivity required, the ease of conjugation with the compound, stability requirements, the available instrumentation and disposal provisions. Non-radioactive labels are often attached by indirect means. A detectable label may be detected by methods known in the art, either directly, for example by direct detection methods, or indirectly, for example by indirect detection methods. Direct and indirect detection methods are well known in the art.

Examples of detection methods include, but are not limited to, fluorescence activated cell sorting (FACS), flow cytometry, immunologically based assay methods from the list of immunohistochemistry, western blotting assay, enzyme-linked immunosorbent assay (ELISA), enzyme-linked immunospot assay (ELISPOT), lateral flow test assay, enzyme immunoassay (EIA), fluorescent polarization immunoassay (FPIA), chemiluminescent immunoassay (CLIA), antibody sandwich capture assay.

Examples of fluorophores suitable for use in some embodiments, for example in some embodiments comprising detection by FACS, are fluorescein derivatives (e.g., fluorescein isothiocyanate (FITC)), phycoerythrin (PE), R-phycoerythrin (rPE) PE I, PE II, PE Texas Red, PE-Cy5, Peridinin chlorophyll protein (PerCP), propidium iodide (PI), PerCP/PI, PerCP-Cy5, PE-Cy7, allophycocyanin (APC), APC-Cy7, Alexa fluor 405, Alexa fluor 430, Alexa fluor 488, Alexa fluor 532, Alexa fluor 546, Alexa fluor 555, Alexa fluor 568, Alexa fluor 594, Alexa fluor 633, Alexa fluor 660, Alexa fluor 680, Pacific Blue, rhodamine, aminocoumarin, hydroxycoumarin, methoxycoumarin, HEX, TRITC, Tamara, 7-Aminoactinomycin D (7-AAD), fluorescent proteins (for example, GFP, YFP, BFP, RFP, also enhanced versions, such as eGFP. eYFP etc.). Other suitable fluorophores will be known to those of skill in the relevant art. Where the label is a fluorescent label, it may be detected by exciting the fluorophore with the appropriate wavelength of light and detecting the resulting fluorescence. The fluorescence may be detected visually, by means of a photographic film, by the use of electronic detectors such as charge coupled devices (CCDs) or photomultipliers and the like.

In embodiments where the label is a radioactive label, means for detection include a scintillation counter, a phosphor detector such as a phosphorimager, or photographic film as in autoradiography.

Detectable labels or entities as provided by some embodiments of the invention can be used to facilitate detection and/or separation of, for example, a cell expressing NY-ESO-1 (e.g. a malignant cell), a cell able to bind a NY-ESO-1 epitope, or a cell able to bind a specific NY-ESO-1 peptide-loaded MHC class II molecule (e.g., a MHC-DRB3*0202 (MHC-DR52b) or MHC-DRB1*0101 (DR1) restricted CD4⁺ T cell), for example isolation or separation from other cells of a cell population, or from a biological sample. Such a cell can be isolated by a variety of methods known in the art, for example, by fluorescence-activated cell sorting (FACS) or magnetic activated cell sorting (MACS).

In some embodiments, detection methods described herein or known in the art may be used to quantify the occurrence of a specific cell type in a subject, for example to quantify the frequency or amount of MHC-DRB3*0202 (MHC-DR52b) or DRB1*0101 (DR1) restricted CD4⁺ T cells able to bind a NY-ESO-1 epitope in a subject. In some embodiments, quantifying may comprise determining cell quantity, frequency, size, proliferation rate, and/or any other quantitative cell parameter known to those of skill in the art. In some embodiments, the detection methods provided herein may be used to monitor a change in a quantitative parameter, for example cell quantity, frequency, and/or proliferation rate, of a specific cell type in a subject over time. In some embodiments, a quantitative parameter, for example, the quantity, frequency, and/or proliferation rate, of a certain cell type, for example, MHC-DRB3*0202 (MHC-DR52b) or DRB1*0101 (DR1) restricted CD4⁺ T cells able to bind a NY-ESO-1 epitope, is monitored in a subject before, during, and/or after the administration of an immunostimulatory composition or peptide, for example a composition comprising an immunostimulatory NY-ESO-1 peptide, for example, NY-ESO-123-137. In some embodiments, a value determined for any quantitative parameter in the subject is compared to a reference, control, or baseline value.

In some embodiments, a CD4+ T-cell specifically binding a peptide-loaded MHC class II molecule is isolated from a cell population, for example, by flow cytometry or by an immunoassay. In some embodiments, the isolated CD4+ T-cell is further characterized, for example, in regard to its phenotype or its function. Assays and reagents as well as biomarkers for phenotypic and/or functional characterization of T-cells are well known to those of skill in the art. Biomarkers for such characterizations include, but are not limited to, T-cell subtype markers (e.g., CD25, CD127, CRTH2), differentiation, adhesion, activation, and migration markers (e.g., CD45RA, CD27, CD28, CCR4, CCR6, CCR7, CXCR3, CD69), and lineage-specific transcription factors (e.g., FOXP3, t-bet, GATA-3, ROR gamma t), as well as cytokine and chemokine markers (e.g., IFN-γ, TNF-α, TGF-β, IL-2, IL-4, IL-5, IL-8, IL-9, IL-10, IL-13, IL 17, IL21, IL-22, IP10, MIP-1alpha, MIP-1beta). An abundance of such markers and marker combinations for phenotypic and/or functional characterization of T-cells are known in the art and include, but are not limited to T-cell migration and/or adhesion markers, for example, ALCAM/CD166, B220/CD45R, CCR1, CCR2, CCR3, CCR4, CCR5, CCR6, CCR7, CCR8, CCR9, CD2AP, CD2F-10/SLAMF9, CD31/PECAM-1, CD43, CD45, CD6, CD81, CD83, CRTH-2, CX3CR1, CXCR1/IL-8 RA, CXCR2/IL-8 RB, CXCR3, CXCR4, CXCR5, CXCR6, Cytohesin-1, DNAM-1, EMMPRIN/CD147, ICAM-1/CD54, ICAM-2/CD102, IGSF8, Integrin alpha 4 beta 1, Integrin alpha 4 beta 7/LPAM-1, Integrin alpha 4/CD49d, Integrin alpha E beta 7, Integrin alpha E/CD 103, Integrin alpha M/CD11b, Integrin alpha X beta 2, Integrin alpha X/CD11c, Integrin beta 2/CD 18, LAIR 1, Leukotriene B4 R1, L-Selectin/CD62L, NCAM-L1, Neprilysin/CD10, PSGL-1, RaplA/B, SIRP gamma/CD172g, Talin1, and TRA-1-85; regulatory T-cell (T reg) markers, for example, 4-1BB/TNFRSF9/CD137, 5′-Nucleotidase/CD73, B220/CD45R, B7-1/CD80, B7-2/CD86, CCR2, CCR4, CCR6, CCR7, CCR8, CD27/TNFRSF7, CD28, CD3, CD3 epsilon, CD30/TNFRSF8, CD38, CD39/ENTPD1, CD4, CD40 Ligand/TNFSF5, CD44, CD45, CD5, CD69, CD8, CD83, Common gamma Chain/IL-2 R gamma, CTLA-4, CXCR3, CXCR4, Fas/TNFRSF6/CD95, FoxP3, Galectin-1, GITR/TNFRSF18, Granzyme A, Granzyme B, HLA-DR, ICAM-1/CD54, IFN-gamma, IGSF2/CD101, IL-10, IL-12/IL-35 p35, IL-2, IL-2 R alpha, IL-2 R beta, IL-4, IL-7 R alpha/CD127, Integrin alpha E beta 7, Integrin alpha E/CD 103, Integrin alpha L/CD11a, Integrin beta 2/CD 18, LAG-3, L-Selectin/CD62L, Neuropilin-1/BDCA4, OX40 Ligand/TNFSF4, OX40/TNFRSF4, PD-1, PDCD6, PRAT4B, P-Selectin/CD62P, RANK/TNFRSF11A, Regulatory T Cells, SLAM/CD150, TGF-beta 1, TLR4, TLR4/MD-2 Complex, TLR7, TRANCE/TNFSF11/RANK L; and T-cell antigen recognition markers, for example, B220/CD45R, B7-1/CD80, B7-2/CD86, CD160, CD1c, CD1d1, CD28, CD3, CD3 epsilon, CD4⁺/45RA⁻, CD4⁺/45RO⁻, CD4⁺/CD62L⁻/CD44, CD4⁺/CD62L⁺/CD44, CD45, CD68/SR-D1, CD8, CD8⁺/45RA⁻, CD8⁺/45RO⁻, Dectin-1/CLEC7A, ILT2/CD85j, ILT3/CD85k, ILT4/CD85d, ILT5/CD85a, ILT6/CD85e, LAG-3, LAX1, Lck, PRAT4B, SIT1, T Cell Receptor alpha Chain-V alpha 24-J alpha Q, TLR1, TLR3, TLR4, TLR4/MD-2 Complex, TRIM, Common gamma Chain/IL-2 R gamma, GM-CSF, GM-CSF R alpha, gp130, IFN-gamma, IFN-gamma R1/CD119, IFN-gamma R2, IL-1 alpha/IL-1F1, IL-1 beta/IL-1F2, IL-1 R1, IL-10, IL-12, IL-12 p70, IL-12 R beta 1, IL-12 R beta 2, IL-12/IL-35 p35, IL-13, IL-13 R alpha 1, IL-17/IL-17A, IL-17A/F Heterodimer, IL-17F, IL-18 R alpha/IL-1 R5, IL-18 R beta/IL-1 R7, IL-18/IL-1F4, IL-2, IL-2 R alpha, IL-23, IL-23 R, IL-24, IL-3, IL-3 R alpha, IL-3 R beta, IL-32, IL-32 alpha, IL-32 gamma, IL-33, IL-4, IL-4 R alpha, IL-5, IL-5 R alpha/CD125, IL-6, IL-6 R alpha, IL-7 R alpha/CD127, NFATC1, NFATC3, Regulatory T Cells, T-bet/TBX21, TCCR/WSX-1, TGF-beta, TGF-beta RI/ALK-5, TGF-beta RII, TGF-beta RIIb, TGF-beta RIII, TNF R1/TNFRSF1A, TNF RII/TNFRSF1B, TNF-alpha/TNFSF1A, TNF-beta/TNFSF1B, TSLP R, and ZAP70.

For an exemplary publication disclosing the use of markers and marker profiles to determine T-cell phenotype and function, see Lee et al., Gene expression profiles during human CD4+ T cell differentiation. Int Immunol. 2004 August; 16(8):1109-24, incorporated herein by reference for disclosure of markers and marker combinations useful for phenotypic and functional T-cell characterization.

In some embodiments, the quantity of a T-cell subtype detected in a cell population is determined, for example, by flow cytometry, and quantitative measures of specific T-cell subtypes are compared to a reference or control level or sample, or to a different sample, for example, a sample from a different subject, or a sample from the same subject, but taken at a different time point. In some embodiments, the further characterization of T-cell populations, and/or of T-cells specifically binding an MHC class II molecule loaded with a peptide of interest are used to determine the reaction of the subject the cell sample is taken from to an administration of an immunostimulatory agent, for example, an immunostimulatory peptide. In some embodiments, a biological sample is obtained from a subject prior to administration of an immunostimulatory peptide to the subject and compared to a sample obtained from the subject after administration of such a peptide. In some embodiments, a biological sample is obtained from a subject diagnosed with or suspected of having a tumor and a T-cell subtype quantity determined in such a sample is compared to a quantity determined in or representative of an equivalent sample obtained from a subject not diagnosed with or suspected to have such a tumor.

In some embodiments, a detection and/or quantification method described herein or known in the art may be used to monitor the induction or enhancement of an immune response in a subject, for example effected by administration of an immunostimulatory NY-ESO-1 peptide or epitope. In some embodiments, an increase in the quantity, frequency, and/or proliferation rate of a certain cell type, for example, MHC-DRB3*0202 (MHC-DR52b) or DRB1*0101 (DR I) restricted CD4⁺ T cells able to bind a NY-ESO-1 epitope, as compared to a reference, control or baseline value, is indicative of induction or enhancement of an immune response, for example, effected by administration of an immunostimulatory NY-ESO-1 peptide.

As used herein, a “subject” may be a human, non-human primate, or other mammal, for example a cow, horse, pig, sheep, goat, dog, cat or rodent.

In some embodiments, the subject is diagnosed to have a tumor or a cancer. In some embodiments, the subject is diagnosed to have a tumor or a cancer expressing NY-ESO-1. In some embodiments, the subject is diagnosed to have, for example, a melanoma, a fibroadenoma, breast cancer, bladder cancer, a sarcoma, ovarian cancer, or a tumor or cancer of a different type. In some embodiments, the subject is diagnosed to carry a type of tumor or a type of cancer known to be associated with NY-ESO-1 expression in a malignant cell or cell type. Methods to diagnose a tumor in a subject are well known in the art. Methods to determine whether a tumor expresses NY-ESO-1 are also well known in the art. In some embodiments, NY-ESO-1 expression may be assessed directly, for example in cases where malignant cells are available from the subject, for example in cases where a tumor biopsy can be or has been obtained. Examples for methods useful for direct assaying NY-ESO-1 expression in malignant cells include, but are not limited to RT-PCR, northern blot, western blot, immunohistochemistry. In some embodiments, indirect assays, for example assays for determining whether a subject has an immune response to NY-ESO-1, may be employed to determine whether a tumor or a malignant cell or cell type expresses NY-ESO-1. Some methods for assaying an immune response to NY-ESO-1 are described herein.

Reference, control, or baseline values can be determined using methods well known to those in the art. For example, a reference, control, or baseline value may be a value from an actual measurement, for example a measurement in the same subject in the absence of or prior to administration of an immunostimulatory epitope of NY-ESO-1, a historical value, an average value obtained from a healthy, non-treated control group of subjects, an empirically determined value, or an arbitrary value.

In some embodiments, a diagnostic method is provided related to the detection of a NY-ESO-1 specific immune response, for example in response to a NY-ESO-1 expressing tumor, in a subject. In some embodiments, the method comprises detecting an immune response against NY-ESO-1 in a subject diagnosed, indicated, or suspected to have a tumor. In some embodiments, the method comprises obtaining a cell population, for example a peripheral blood cell population, from the subject and determining whether NY-ESO-1 specific, DRB3*0202 (DR52b) or DRB1*0101 (DR1) restricted CD4⁺ T-cells are present, wherein if such cells are present in the subject, the subject is indicated to have an immune response against NY-ESO-1. In some embodiments, the subject is a subject known to have a tumor, for example a melanoma, a fibroadenoma, breast cancer, bladder cancer, a sarcoma, ovarian cancer, or a tumor or cancer of a different type known to express NY-ESO-1, and, if an anti-NY-ESO-1 immune response is detected in the subject, the subject is indicated to have a tumor expressing NY-ESO-1.

The invention involves the use of various materials disclosed herein to induce or enhance an immune response in subjects. As used herein, “inducing or enhancing an immune response” means increasing or activating an immune response against an antigen. It does not require elimination or eradication of a condition but rather contemplates the clinically favorable enhancement of an immune response toward an antigen. Generally accepted animal models, can be used for testing of immunization approaches against cancer using a NY-ESO-1 molecule of the invention. For example, human cancer cells can be introduced into a mouse to create a tumor, and one or more NY-ESO-1 molecules of the invention can be delivered by the methods described herein. The effect on the cancer cells (e.g., reduction of tumor size) can be assessed as a measure of the effectiveness of the NY-ESO-1 molecule administration. Of course, testing of the foregoing animal model using more conventional methods for inducing an immune response include the administration of one or more NY-ESO-1 polypeptides or fragments derived therefrom, optionally combined with one or more adjuvants and/or cytokines to boost the immune response.

Methods for inducing an immune response, including formulation of an immunizing composition and selection of doses, route of administration and the schedule of administration (e.g. primary and one or more booster doses), are well known in the art. The tests also can be performed in humans, where the end point is to test for the presence of enhanced levels of circulating CTLs against cells bearing the antigen, to test for levels of circulating antibodies against the antigen, to test for the presence of cells expressing the antigen and so forth.

As part of the immune response-inducing or enhancing compositions of the invention, one or more substances that potentiate an immune response may be administered along with the peptides described herein. Such substances include adjuvants and cytokines. An adjuvant is a substance incorporated into or administered with antigen that potentiates the immune response. Adjuvants may enhance the immunological response by providing a reservoir of antigen (extracellularly or within macrophages), activating macrophages and stimulating specific sets of lymphocytes. Adjuvants of many kinds are well known in the art. Specific examples of adjuvants include Montanide, ISA51, CpG 7909 (9), immunostimulatory nucleic acid molecules, e.g. CpG oligonucleotides (see e.g. Kreig et al., Nature 374:546-9, 1995); monophosphoryl lipid A (MPL, SmithKline Beecham), a congener obtained after purification and acid hydrolysis of Salmonella minnesota Re 595 lipopolysaccharide; saponins including QS21 (SmithKline Beecham), described in PCT application WO96/33739 (SmithKline Beecham), ISCOM (CSL Ltd., Parkville, Victoria, Australia) derived from the bark of the Quillaia saponaria molina tree, QS-7, QS-17, QS-18, and QS-L1 (So et al., Mol. Cells. 7:178-186, 1997); incomplete Freund's adjuvant; complete Freund's adjuvant; alum; various water-in-oil emulsions prepared from biodegradable oils such as squalene and/or tocopherol; and factors that are taken up by the so-called ‘toll-like receptor 7’ on certain immune cells that are found in the outside part of the skin, such as imiquimod (3M, St. Paul, Minn.). Preferably, the antigens are administered mixed with a combination of DQS21/MPL. The ratio of DQS21 to MPL typically will be about 1:10 to 10:1, preferably about 1:5 to 5:1 and more preferably about 1:1. Typically for human administration, DQS21 and MPL will be present in a vaccine formulation in the range of about 1 μg to about 100 μg. Other adjuvants are known in the art and can be used in the invention (see, e.g. Goding, Monoclonal Antibodies: Principles and Practice, 2nd Ed., 1986). Methods for the preparation of mixtures or emulsions of polypeptide and adjuvant are well known to those of skill in the art of inducing and/or enhancing an immune response and the art of vaccination.

Other agents which may aid in inducing or enhancing an immune response of may also be administered to the subject. For example, other cytokines are also useful in vaccination protocols as a result of their lymphocyte regulatory properties. Many other cytokines useful for such purposes will be known to one of ordinary skill in the art, including interleukin-12 (IL-12) which has been shown to enhance the protective effects of vaccines (see, e.g., Science 268: 1432-1434, 1995), GM-CSF and IL-18. Thus cytokines can be administered in conjunction with antigens and adjuvants to increase the immune response to the antigens. There are a number of additional immune response potentiating compounds that can be used in vaccination protocols. These include costimulatory molecules provided in either protein or nucleic acid form. Such costimulatory molecules include the B7-1 and B7-2 (CD80 and CD86 respectively) molecules which are expressed on dendritic cells (DC) and interact with the CD28 molecule expressed on the T cell. This interaction provides costimulation (signal 2) to an antigen/MHC/TCR stimulated (signal 1) T cell, increasing T cell proliferation and effector function. B7 also interacts with CTLA4 (CD152) on T cells and studies involving CTLA4 and B7 ligands indicate that the B7-CTLA4 interaction can enhance antitumor immunity and CTL proliferation (Zheng et al., Proc. Nat'l Acad. Sci. USA 95:6284-6289, 1998).

B7 typically is not expressed on tumor cells so they are not efficient antigen presenting cells (APCs) for T cells. Induction of B7 expression would enable the tumor cells to stimulate more efficiently CTL proliferation and effector function. A combination of B7/IL-6/IL-12 costimulation has been shown to induce IFN-gamma and a Th1 cytokine profile in the T cell population leading to further enhanced T cell activity (Gajewski et al., J. Immunol. 154:5637-5648, 1995). Tumor cell transfection with B7 has been discussed in relation to in vitro CTL expansion for adoptive transfer immunotherapy by Wang et al. (J. Immunother. 19:1-8, 1996). Other delivery mechanisms for the B7 molecule would include nucleic acid (naked DNA) immunization (Kim et al., Nature Biotechnol. 15:7:641-646, 1997) and recombinant viruses such as adeno and pox (Wendtner et al., Gene Ther. 4:726-735, 1997). These systems are all amenable to the construction and use of expression cassettes for the coexpression of B7 with other molecules of choice such as the antigens or fragment(s) of antigens discussed herein (including polytopes) or cytokines. These delivery systems can be used for induction of the appropriate molecules in vitro and for in vivo vaccination situations. Anti-CD28 antibodies to directly stimulate T cells in vitro and in vivo could also be used. Similarly, the inducible co-stimulatory molecule ICOS which induces T cell responses to foreign antigen could be modulated, for example, by use of anti-ICOS antibodies (Hutloff et al., Nature 397:263-266, 1999).

Lymphocyte function associated antigen-3 (LFA-3) is expressed on APCs and some tumor cells and interacts with CD2 expressed on T cells. This interaction induces T cell IL-2 and IFN-gamma production and can thus complement but not substitute, the B7/CD28 costimulatory interaction (Parra et al., J. Immunol., 158:637-642, 1997; Fenton et al., J. Immunother., 21:95-108, 1998).

Lymphocyte function associated antigen-1 (LFA-1) is expressed on leukocytes and interacts with ICAM-1 expressed on APCs and some tumor cells. This interaction induces T cell IL-2 and IFN-gamma production and can thus complement but not substitute, the B7/CD28 costimulatory interaction (Fenton et al., 1998). LFA-1 is thus a further example of a costimulatory molecule that could be provided in a vaccination protocol in the various ways discussed above for B7.

Complete CTL activation and effector function requires Th cell help through the interaction between the Th cell CD40L (CD40 ligand) molecule and the CD40 molecule expressed by DCs (Ridge et al., Nature 393:474, 1998; Bennett et al., Nature 393:478, 1998; Schoenberger et al., Nature 393:480, 1998). This mechanism of this costimulatory signal is likely to involve upregulation of B7 and associated IL-6/IL-12 production by the DC (APC). The CD40-CD40L interaction thus complements the signal 1 (antigen/MHC-TCR) and signal 2 (B7-CD28) interactions.

The use of anti-CD40 antibodies to stimulate DC cells directly, would be expected to enhance a response to tumor associated antigens which are normally encountered outside of an inflammatory context or are presented by non-professional APCs (tumor cells). Other methods for inducing maturation of dendritic cells, e.g., by increasing CD40-CD40L interaction, or by contacting DCs with CpG-containing oligodeoxynucleotides or stimulatory sugar moieties from extracellular matrix, are known in the art. In these situations Th help and B7 costimulation signals are not provided. This mechanism might be used in the context of antigen pulsed DC based therapies or in situations where Th epitopes have not been defined within known tumor associated antigen precursors.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents.

In some embodiments, a composition containing the agents provided herein is provided. The composition may comprise any of the peptides, epitopes, optionally peptide-loaded MHC class II molecules, MHC class II monomers, multimers, and/or tetramers, ligands, binding molecules, and/or detectable labels provided herein. For example, a composition may comprise a diagnostic and/or therapeutic agent, for example an immunostimulatory NY-ESO-1 epitope, and/or a peptide-loaded MHC class II molecule, in an optional pharmaceutically acceptable carrier. In some embodiments, a method for forming a medicament that involves placing a therapeutically effective amount of a therapeutic agent in a pharmaceutically acceptable carrier to form one or more doses is provided. The effectiveness of a treatment or prevention method of the invention can be determined using standard diagnostic methods described herein.

Therapeutic compositions of the present invention are administered in pharmaceutically acceptable preparations. Such preparations may contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Examples of physiologically and pharmaceutically acceptable carriers include, without being limited to, diluents, fillers, salts, buffers, stabilizers, solubilizers, and other materials which are well known in the art. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

Therapeutics according to some embodiments of the invention can be administered by any conventional route, for example injection or gradual infusion over time. The administration may, for example, be oral, intravenous, intratumoral, intraperitoneal, intramuscular, intracavity, subcutaneous, or transdermal.

The compositions of some embodiments of the invention are administered in effective amounts. An “effective amount” is that amount of a composition that alone, or together with further doses, produces a desired response, for example, an increase in the number, frequency and/or proliferation rate of MHC-DRB3*0202 (MHC-DR52b) or DRB1*0101 (DR1) restricted CD4⁺ T cells able to bind a NY-ESO-1 epitope in a subject, for example a subject diagnosed with a tumor expressing NY-ESO-1, or a reduction in size or growth or inhibition of proliferation of a tumor or malignant cells, for example a tumor or malignant cells expressing NY-ESO-1, in a subject. In some cases of treating a particular disease or condition characterized by the presence of cells expressing NY-ESO-1, the desired response is inhibiting the progression of the disease. This may involve slowing the progression of the disease temporarily, although more preferably, it involves halting the progression of the disease permanently. In some cases, the desired response to treatment is a permanent effect, for example a return to a state comparable to those found in healthy individuals. In some cases, the desired response to treatment can be delaying or preventing the manifestation of clinical symptoms characteristic for the disease or condition.

The effect of treatment can be monitored by routine methods or can be monitored according to a diagnostic method of the invention discussed herein, for example a method using NY-ESO-1 peptide-loaded MHC class II multimers to detect and/or quantify MHC-DRB3*0202 (MHC-DR52b) or DRB1*0101 (DR1) restricted CD4⁺ T cells able to bind a NY-ESO-1 epitope in a subject.

The effective amount may depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Pharmaceutical compositions according to some embodiments of this invention preferably are sterile and contain an effective amount of one or more therapeutic agents as described herein for producing the desired response in a unit of weight or volume suitable for administration to a patient. The response can, for example, be measured by determining any of the quantitative parameters described herein. Other parameters and suitable assays to determine the response will be evident to those of skill in the art.

The doses of one or more therapeutic agents as described herein (e.g., polypeptide, peptide-loaded MHC class II molecule, multimer, tetramer, etc.) administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

Administration of polypeptide compositions to mammals other than humans, e.g. for testing purposes or veterinary therapeutic purposes, is carried out under substantially the same conditions as described above.

The pharmaceutical compositions may contain suitable buffering agents, for example acetic acid in a salt, citric acid in a salt, boric acid in a salt, and/or phosphoric acid in a salt.

The pharmaceutical compositions also may contain, optionally, suitable preservatives, such as: benzalkonium chloride, chlorobutanol, parabens and/or thimerosal.

The pharmaceutical compositions may conveniently be presented in unit dosage form and may be prepared by any of the methods well-known in the art of pharmacy.

All methods may include the step of bringing the active agent into association with a carrier which constitutes one or more accessory ingredients. In general, compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other examples of compositions include suspensions in aqueous liquids or non-aqueous liquids such as a syrup, elixir or an emulsion. Examples of compositions for parenteral administration include, without being limited to, sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Examples of aqueous carriers are water, alcoholic/aqueous solutions, emulsions or suspensions, for example saline and buffered media. Examples of parenteral vehicles are sodium chloride solution, Ringer's dextrose, dextrose and sodium chloride, and lactated Ringer's or fixed oils. Examples for intravenous vehicles are fluid and nutrient replenishers, electrolyte replenishers (such as those based on Ringer's dextrose), and the like. Preservatives and other additives may also be present such as, for example, antimicrobials, anti-oxidants, chelating agents, and inert gases, and the like.

The pharmaceutical agents of some embodiments of the invention may be administered alone, in combination with each other, and/or in combination with other drug therapies and/or treatments. Examples of therapies and/or treatments may include, but are not limited to: surgical intervention, chemotherapy, radiotherapy, and adjuvant systemic therapies.

In some embodiments, the invention also provides one or more kits comprising one or more containers comprising one or more of the pharmaceutical compounds or agents of the invention. In some embodiments, a diagnostic kit is provided that includes an isolated immunostimulatory NY-ESO-1 peptide, and/or an isolated MHC class II multimer or tetramer, optionally loaded with a NY-ESO-1 peptide. In some embodiments, a kit is provided that further includes other reagents necessary or useful for performing a method described herein. Examples for such reagents are a detectable label, a labeling reagent, a detection reagent, and a buffering reagent. Diagnostic kits provided herein are, for example, useful for determining the presence and/or expression of a MHC DRB3*0202 or a DRB1*0101 allele and/or the presence of MHC DRB3*0202 (DR52b) or DRB1*0101 (DR1) restricted T cells in a subject. This information can be used as the basis for a clinical diagnosis, the selection of a clinical course of action, and/or for basic research purposes.

Additional materials may be included in any or all kits of the invention, and such materials may include, but are not limited to, for example, buffers, water, enzymes, tubes, control molecules, etc. One or more kits may also include instructions for the use of the one or more pharmaceutical compounds or agents of the invention, for example for the treatment of a tumor or a cancer, or the diagnostic methods described herein.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all methods, reagents, and configurations described herein are meant to be exemplary and that the actual methods, reagents, and configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the embodiments described herein are presented by way of example only and that, within the scope of the appended claims and equivalents thereto, the invention may be practiced otherwise than as specifically described and claimed. The present invention is directed to each individual feature, system, article, material, reagent, kit, and/or method described herein. In addition, any combination of two or more such features, systems, articles, materials, kits, and/or methods, if such features, systems, articles, materials, reagents, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an”, as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B”, when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of”, when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one act, the order of the acts of the method is not necessarily limited to the order in which the acts of the method are recited.

### EXAMPLES

### Example 1

Patients Samples, Cells and Tissue Culture.

Peripheral blood samples were collected from cancer patients enrolled in a clinical trial of vaccination with recombinant ESO protein, Montanide ISA51 and CpG 7909 (9) upon informed consent and approval by the Institutional Review Boards of Columbia University and New York University medical centers. Study patients received 4 subcutaneous injections of rESO/Montanide/CpG vaccine at 3-week intervals. Patients enrolled had histological diagnosis of cancer types known to express ESO. Of the 18 patients enrolled in the clinical trial, 11 were diagnosed with melanoma, 3 with breast cancer, 3 with sarcoma and 1 with ovarian cancer. At study entry 1 sarcoma patient had a lung metastasis and all other patients had no evidence of disease. With the exception of 1 melanoma patient, none of the patients had detectable ESO-specific immune responses prior to vaccination, but they all developed specific antibody and CD4⁺ T cell responses following vaccination, as reported previously (9). Peripheral blood samples from healthy donors were obtained from the Etablissement Francais du Sang Pays de la Loire (Nantes, France). MHC class II alleles were determined by high resolution molecular typing. Melanoma cell lines were kindly provided by Dr. D. Rimoldi (Ludwig Institute for Cancer Research, Lausanne, Switzerland) and Prof. F. Jotereau (INSERM U892, Nantes, France). Monocyte-derived dendritic cells (moDC) were generated from enriched CD14⁺ cells, isolated from PBMC using magnetic sorting (Miltenyi Biotech Inc., Bergisch Gladbach, Germany), by culture in the presence of 1000 U/ml rhGM-CSF and 1000 U/ml rhIL-4 (R&D Systems, Minneapolis, Minn.) for 5 days.

Assessment of ESO-Specific CD4⁺ T Cell Responses and Generation of Specific Clones.

For ex-vivo assessment, cryopreserved total PBMC were thawed, rested overnight, and stimulated for 7 hours in the absence or presence of a pool of 20 to 24 amino acid long overlapping peptides (NeoMPS Inc., San Diego, Calif.) spanning the full length ESO sequence. Brefeldin A was added 2 hours after the beginning of the incubation. Cells were then stained with antibodies directed against surface markers (CD3, CD4 and CD8 (BD Biosciences, San Josa, Calif.)), fixed, permeabilized and stained with anti-IFN-γ, —IL-4, IL-10 (BD Biosciences) or -IL17 mAb (eBiosciences, San Diego, Calif.), as previously described (9). For assessment of CD4⁺ T cell responses following in vitro stimulation, CD4⁺ cells were enriched from PBMC by magnetic cell sorting (Miltenyi Biotec Inc.) and stimulated with irradiated autologous APC in the presence of the NY-ESO-1 peptide pool or the indicated NY-ESO-1 peptides (2 μM each, NeoMPS Inc.), rhIL-2 (10 IU/ml) and rhIL-7 (10 ng/ml). At day 8 cultures were tested for intracellular IFN-γ secretion following stimulation, during 4 hours, in the absence or presence of the ESO peptide pool or of individual peptides. Where indicated, CD4⁺ T cell cultures were pre-incubated during 1 h with anti-HLA-DRw52 mAb (clone 7.3.19.1, Monosan, Uden, The Netherlands) prior to peptide stimulation. ESO₁₁₉₋₁₄₃-specific CD4⁺ T cells were isolated, following 4 h stimulation, using the IFN-γ secretion assay—cell detection kit (Miltenyi Biotech Inc.) and flow cytometry cell sorting and cloned by limiting dilution cultures in the presence of phytohemagglutinin, allogeneic irradiated PBMC and rhIL-2 (100 U/ml). Clones were subsequently expanded by periodic stimulation (every 3-4 weeks) under the same conditions.

Antigen Recognition Assays and TCR BV Analysis.

CD4⁺ T cell clones were stimulated in the absence or presence of peptides, at the indicated concentration, and IFN-γ production was assessed either in a 4 h intracellular cytokine staining assay as described above or by measurement of IFN-γ in 24 h culture supernatant by ELISA as previously described (7). Where indicated, EBV-B cell lines or PBMC from healthy donors were pre-incubated in the absence or presence of peptide ESO₁₁₉₋₁₄₃, washed and used to stimulate CD4⁺ T cell clones. Blocking experiments were performed by pre-incubating CD4⁺ T cells with anti-HLA-DR (clone G46-6, BD Biosciences), —HLA-DP (clone B7/21, Abcam, Cambridge, United Kingdom), -DQ (clone SPVL3, Immunotech, Marseille, France) or -DRw52 mAb, prior to peptide stimulation. For assessment of reactivity to naturally processed full-length ESO, tumor cell lines or moDC were either incubated during 16 h with recombinant ESO or Melan-A proteins or transfected by electroporation with ESO-encoding pcDNA3.1 vector (Amaxa Inc., Walkersville, Md.) and used to stimulate CD4⁺ T cell clones. TCR variable β chain (BV) usage was determined by flow cytometry using anti-BV mAb (Immunotech) and by molecular analysis as described previously (10) using a panel of previously validated primers (11) and nomenclature according to Arden B. et al. (12).

Isolation of ESO₁₁₉₋₁₄₃-Specific Vaccine-Induced T_{H}1 Clones.

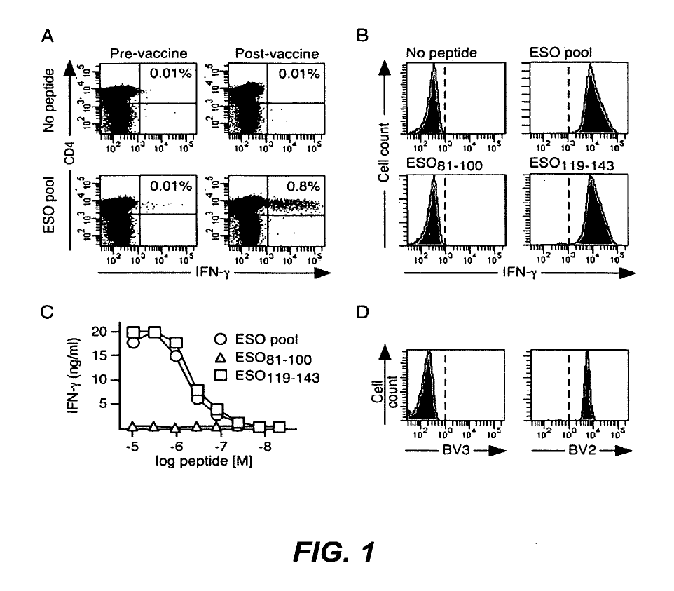

Following vaccination with rESO, all patients developed a specific CD4⁺ T cell response (9). For patient C2, vaccine-induced IFN-γ-producing CD4⁺ T cells were detected in post-immune but not in pre-immune PBMC in response to stimulation with a pool of overlapping peptides covering ESO (FIG. 1A). ESO-specific IFN-γ-producing CD4⁺ T cells represented ex-vivo close to 1% of total CD4⁺ T cells and displayed a typical T_{H}1 profile, as they failed to produce IL-4, IL-17 or IL-10 in response to antigen stimulation (data not shown). We isolated specific CD4⁺ T cells based on IFN-γ secretion, followed by cloning under limiting dilution conditions as described (2). We obtained four clones reactive to the ESO peptide pool and tested them for reactivity to immunodominant peptides ESO₈₁₋₁₀₀ and ESO₁₁₉₋₁₄₃. The clones specifically recognized peptide ESO₁₁₉₋₁₄₃, but not ESO₈₁₋₁₀₀ (FIGS. 1B and 1C). As determined by using a panel of TCR variable 0 chain (BV)-specific mAb, all clones used BV2 (FIG. 1D).

Peptide ESO₁₁₉₋₁₄₃ is Recognized by Vaccine-Induced CD4⁺ T Cells in the Context of HLA-DR52b.

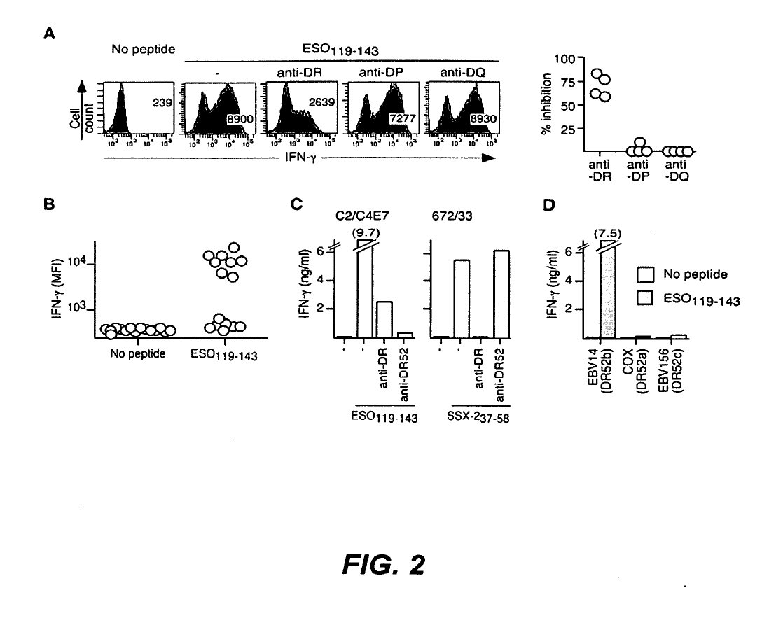

To determine the HLA-restriction of vaccine-induced ESO₁₁₉₋₁₄₃-specific clones from patient C2, we first assessed inhibition of antigen recognition using blocking mAb against HLA-DR, -DP and -DQ. For all clones, antigen recognition was inhibited in the presence of anti-DR but not of anti-DP and -DQ mAb (FIG. 2A). As assessed by high resolution molecular typing, patient C2 expresses DRB1*0701, DRB1*1201, DRB3*0202 and DRB4*0103 alleles. We first tested the clones for their capacity to recognize peptide ESO₁₁₉₋₁₄₃ presented by transfected mouse fibroblasts expressing DRB1*0701 (L-DR7 cells), and detected no reactivity (data not shown). We could not directly assess restriction by DRB1*1201 as no DRB1*1201⁺ APC were available. To establish the frequency of the restricting allele in the population, we assessed the ability of antigen presenting cells from HLA-unselected healthy donors to present peptide ESO₁₁₉₋₁₄₃ to clone C2/C4E7. This analysis revealed that APC from 8/15 donors were able to present the antigen (FIG. 2B). Therefore, the frequency of the restricting allele (50%) did not correspond to the frequency of DRB1*1201 in the population (about 3%), leaving DRB3 and DRB4 molecules, that are less polymorphic than DRB1, as possible candidates. In line with this, a monoclonal antibody specific for HLA-DR52 abrogated antigen recognition by clone C2/C4E7 but not by a control CD4⁺ T cell clone (672/33) recognizing an unrelated peptide (SSX-2₃₇₋₅₈) in the context of DR11 (13) (FIG. 2C). To define the restricting allele, we used as APC molecularly typed EBV-immortalized B cell lines EBV14 (DRB3*0202 (DR52b)), COX (DRB3*0101 (DR52a)) and EBV156 (DRB3*0301 (DR52c)). CD4⁺ T cells recognized peptide ESO₁₁₉₋₁₄₃ presented by EBV14, but not by COX or EBV156, thus establishing DRB3*0202 (DR52b) as the restricting allele (FIG. 2D).

ESO₁₂₃₋₁₃₇ is the Minimal Optimal Peptide Recognized by DR52b-Restricted CD4⁺ T Cell Clones.

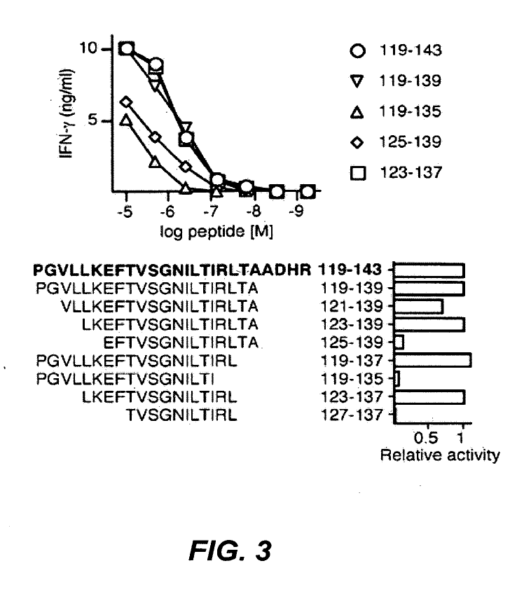

To define the DR52b epitope within the ESO₁₁₉₋₁₄₃ region, we used the MHC class II peptide prediction algorithm RankPep (imed.med.ucm.es/Tools/rankpep.html) to identify ESO sequences with significant predicted binding capacity to DRB3*0202. Only two 9-mer core sequences were identified (Table 3). In particular, peptide ESO₁₂₇₋₁₃₅ was predicted to bind DR52b with an affinity only 3 folds inferior to that of the consensus sequence. Based on the identification of ESO₁₂₇₋₁₃₅ as the putative core region, we designed truncated peptides by sequential removal of amino acids at either the N- or C-terminus of the original 24-mer and assessed their relative recognition efficiency by peptide titration (FIG. 3). Removal of amino acids up to position 123 at the N-terminus did not significantly affect recognition, whereas further truncation significantly decreased recognition. At the C-terminus, truncation up to position 137 did not affect recognition, whereas further truncation decreased it. These results identified the 15-mer ESO₁₂₃₋₁₃₇ as the minimal optimal peptide recognized by DR52b-restricted CD4⁺ T cells.

TABLE 3
Ranking and score of putative ESO sequences
predicted to bind DR52b

| | Posi- | | | | |
|---|---|---|---|---|---|
| Rank* | tion | Sequence | Score* | % Optimal† | SEQ ID NO: |
| 1 | 127 | TVSGNILTI | 17.64 | 31.95 | 59 |
| 2 | 138 | TAADHRQLQ | 1.838 | 3.33 | 82 |

*Ranking and score were calculated using the binding prediction algorithm RankPep (imed.med.ucm.es/Tools/rankpep.html). † % Optimal = (score indicated peptide/score consensus reference sequence YIKGNRKPI, SEQ ID NO: 120) × 100.

DR52b-Restricted CD4⁺ T Cell Clones Recognize Natural ESO Antigen Exogenously Processed by APC.

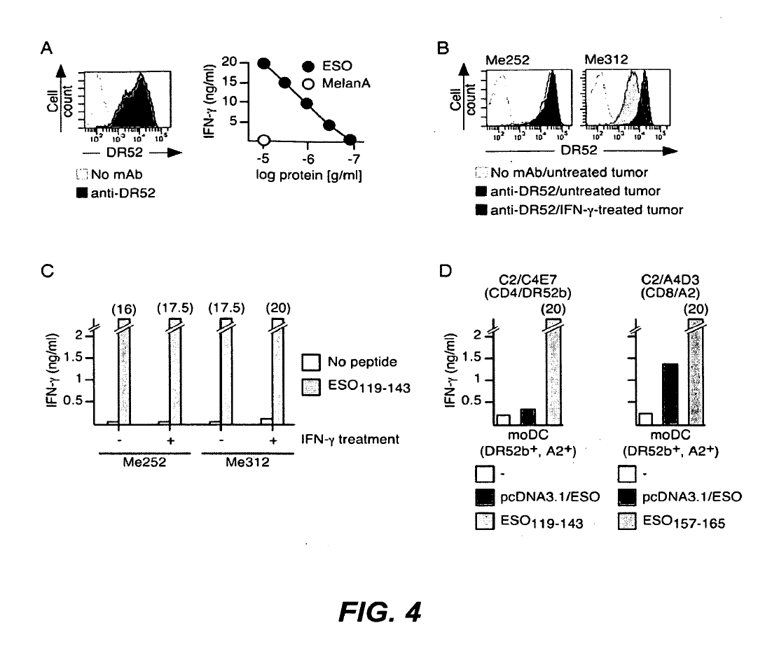

To assess the recognition of natural ESO antigen by DR52b-restricted CD4⁺ T cells, we tested their ability to recognize rESO processed by professional APC (DR52b⁺ monocyte-derived dendritic cells, moDC (FIG. 4A, left panel)). MoDC efficiently processed rESO and presented the DR52b-restricted epitope to specific CD4⁺ T cells (FIG. 4A, right panel). To assess if DR52b-restricted CD4⁺ T cells could also directly recognize the ESO antigen endogenously expressed by tumor cells, we selected two ESO⁺ DR52b⁺ melanoma cells lines (Me252 and Me312) (14). Both lines expressed significant levels of DR52 (FIG. 4B) and presented peptide ESO₁₁₉₋₁₄₃ to specific CD4⁺ T cells (FIG. 4C). However, we failed to detect significant direct recognition of tumor cells by ESO-specific DR52b-restricted CD4⁺ T cells even after treatment with IFN-γ (FIG. 4C). Similarly, A2⁺DR52b⁺ moDC, transfected with a plasmid encoding ESO, failed to be recognized by specific DR52b-restricted CD4⁺ T cells, although they were recognized by A2-restricted CD8⁺ T cells (FIG. 4D). Thus, ESO₁₁₉₋₁₄₃-specific DR52b-restricted CD4⁺ T cells were able to recognize exogenously but not endogenously processed ESO antigen.

ESO₁₁₉₋₁₄₃-Specific DR52b-Restricted CD4⁺ T Cell Responses are Immunodominant Following Vaccination with ESO Protein.

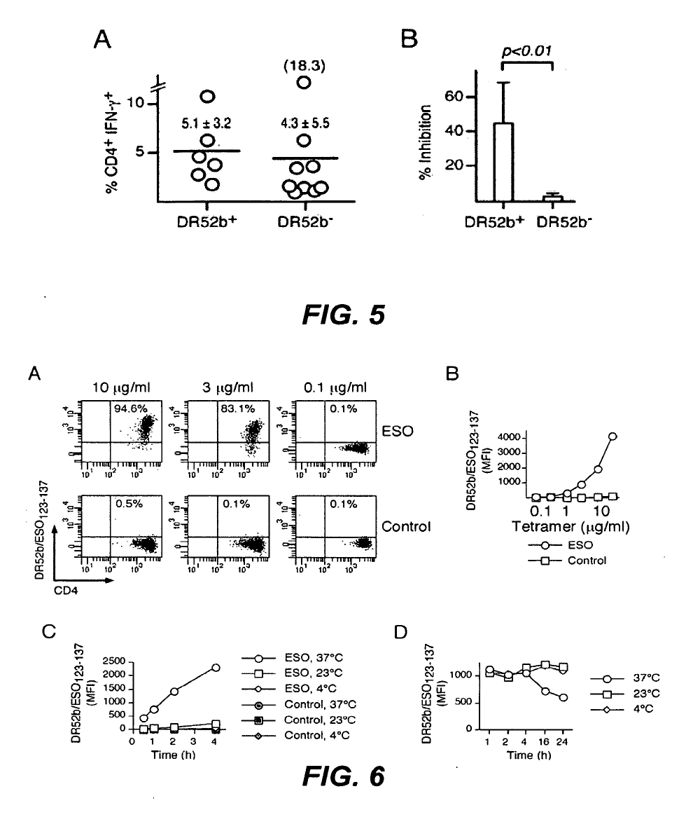

To evaluate the prevalence of ESO-specific DR52b-restricted CD4⁺ T cell responses in patients vaccinated with rESO, we isolated CD4⁺ T cells from post-immune samples of 15 patients and stimulated them during 10 days with the pool of ESO peptides. We then assessed the presence of specific CD4⁺ T cells by intracellular IFN-γ staining after stimulation with peptide ESO₁₁₉₋₁₄₃. To determine the proportion of DR52-restricted CD4⁺ T cells in the cultures, we performed the assay in the absence or presence of anti-DR52 specific antibody. Six of the analyzed patients expressed DR52b and 9 were negative. Significant proportions of CD4⁺ T cells specifically producing IFN-γ in response to ESO₁₁₉₋₁₄₃ were detected in post-vaccine samples from all patients (FIG. 5A). Their frequency in cultures from different patients ranged from 1.8 to 18.3% of CD4⁺ T cells and was similar, in average, for DR52b⁺ and DR52b⁻ patients. However, whereas no significant inhibition of antigen recognition was observed for cultures from DR52b⁻ patients in the presence of the anti-DR52 mAb, the latter blocked antigen recognition by ESO₁₁₉₋₁₄₃ specific CD4⁺ T cells in cultures from all DR52b⁺ patients, to different extents (21-88%, mean 44.7%±23.8%) (FIG. 5B). Together, our results show that DR52b-restricted ESO₁₁₉₋₁₄₃-specific CD4⁺ T cell responses are immunodominant in DR52b-expressing patients vaccinated with the rESO.

Conserved TCR Usage of ESO₁₁₉₋₁₄₃-Specific DR52b-Restricted CD4⁺ T Cell Clones.

T cell clones recognizing defined MHC/peptide complexes can display conserved structural features. To assess TCR usage of clones recognizing peptide ESO₁₁₉₋₁₄₃ in the context of DR52b, we derived a panel of ESO₁₁₉₋₁₄₃-specific clones from vaccinated patients expressing DR52b. We obtained 62 clones from 4 different patients (50 clones from patient C2, 8 from patients N13, 3 from C5 and 1 from patient N10). Of those, 33 (53%) were DR52b-restricted, as determined by using molecularly typed APC (data not shown). Because the CD4⁺ T cell clones initially obtained from patient C2 used BV2, we assessed BV2 expression by all other clones using specific mAb. This analysis revealed that over 70% of the DR52b-restricted clones (including clones from 3 patients) expressed BV2. To further assess the structural features of DR52b-restricted TCR, we sequenced the TCR β chains of the BV2-expressing clones. We identified 13 distinct clonotypes (Table 4), 5 of which used the same BJ (2.1) whereas the remaining 8 used 6 other BJ. In addition, the 13 distinct CDR3 regions were variable both in terms of length (10-13 amino acids) and amino acid composition. Some conservation was nevertheless noticeable, such as the presence of A at position 1 of the CDR3 of 11 of the 13 clonotypes and R at position 2 for 7 of them.

TABLE 4
Analysis of CDR3 β sequence and length of BV2+ ESO₁₁₉₋₁₄₃-specific
DR52b-restricted CD4+ T cell clones.

| | Number | | | | |
|---|---|---|---|---|---|
| | of | | | | SEQ ID |
| Patient | clones | BV* | CDR3β | BJ | NO: |
| C2 | 7 | BV2 | ICS A N N R A R G S Y N E Q FFG | 2.1 | 61 |
| | 3 | BV2 | ICS A F R R T D G D T Q YFG | 2.3 | 62 |
| | 3 | BV2 | ICS A R D M G T A E V Y G Y TFG | 1.2 | 63 |
| | 2 | BV2 | ICS V A S R R E G E E Q YFG | 2.7 | 64 |
| | 1 | BV2 | ICS A R D E R G G R Y N E Q FFG | 2.1 | 65 |
| | 1 | BV2 | ICS A Y P G V T N E K L FFG | 1.4 | 66 |
| | 1 | BV2 | ICS A S S P G T S G R A G E L FFG | 2.2 | 67 |
| | 1 | BV2 | ICS A R G G L P S S Y N E Q FFG | 2.1 | 68 |
| | 1 | BV2 | ICS A R D P S K S S Y N E Q FFG | 2.1 | 69 |
| N10 | 1 | BV2 | ICS A R G P G Q G I G D T Q YFG | 2.3 | 70 |
| N13 | 1 | BV2 | ICS A R G A G N T G E L FFG | 2.2 | 71 |
| | 1 | BV2 | ICS L I R A D T N T E A FFG | 1.1 | 72 |
| | 1 | BV2 | ICS A R G A S G A N Y N E Q FFG | 2.1 | 73 |

*Nomenclature used is according to Arden B. et al. (12).

### Production of HLA-DR52b

DNA Constructs for DR52b Beta Chain and DR Alpha Chain.

All constructs were prepared on the basis of the pMT\BiP\V5-His A vector (Invitrogen). The extracellular parts of MHC class II chains were cloned between the BglII and EcoRI sites, joined by the acidic/basic leucine zipper between EcoRI and EcoRV sites. The stop codon preceded the EcoRV site. The DR alpha chain carries the acidic zipper and the beta chain the basic, followed by an Avi-Tag (also called BSP sequence) (Avidity). A flexible glycine-serine linkers (e.g. G-[SG]₂) were used to connect the MHC class II extracellular part to the leucine zippers and the basic leucine zipper to the Avi-Tag.

The extracellular part of DR52b beta was amplified from cDNA prepared from DR52b⁺ EBVB cells using the following primers: DR4beta forward BglII: CTTTAGATCTCGACCACGTTTCTTGGAGC (SEQ ID NO: 74); DR4beta reverse EcoRI: CTTTGAATTCCTTGCTCTGTGCAGATTCAG (SEQ ID NO: 75).

DR alpha was amplified from a plasmid containing the cloned full length chain in pCR3 plasmid (Roetzschke/Falk lab) using following primers: DR alpha forward BglII: CTTTAGATCTatcaaagaagaacatgtgATC (SEQ ID NO: 76); DR alpha reverse EcoRI: CTTCGAATTCGTTCTCTGTAGTCTCTGGG (SEQ ID NO: 77).

The DNA and protein sequences of the complete constructs are as follows:

DR alpha ALZ
(SEQ ID NO: 78)
AGATCTATCAAAGAAGAACATGTGATCATCCAGGCCGAGTTCTATCTGAATCCTGACCAATCAGGCGAGTTTAT
GTTTGACTTTGATGGTGATGAGATTTTCCATGTGGATATGGCAAAGAAGGAGACGGTCTGGCGGCTTGAAGAAT
TTGGACGATTTGCCAGCTTTGAGGCTCAAGGTGCATTGGCCAACATAGCTGTGGACAAAGCCAACCTGGAAATC
ATGACAAAGCGCTCCAACTATACTCCGATCACCAATGTACCTCCAGAGGTAACTGTGCTCACGAACAGCCCTGT
GGAACTGAGAGAGCCCAACGTCCTCATCTGTTTCATCGACAAGTTCACCCCACCAGTGGTCAATGTCACGTGGC
TTCGAAATGGAAAACCTGTCACCACAGGAGTGTCAGAGACAGTCTTCCTGCCCAGGGAAGACCACCTTTTCCGC
AAGTTCCACTATCTCCCCTTCCTGCCCTCAACTGAGGACGTTTACGACTGCAGGGTGGAGCACTGGGGCTTGGA
TGAGCCTCTTCTCAAGCACTGGGAGTTTGATGCTCCAAGCCCTCTCCCAGAGACTACAGAGAACGAATTCGGTG
GTGGATCAGGAGGTTCAACTACAGCTCCATCAGCTCAGCTCGAAAAAGAGCTCCAGGCCCTGGAGAAGGAAAAT
GCACAGCTGGAATGGGAGTTGCAAGCACTGGAAAAGGAACTGGCTCAGTAA
DR alpha ALZ
(SEQ ID NO: 79)
RSIKEEHVIIQAEFYLNPDQSGEFMFDFDGDEIFHVDMAKKETVWRLEEFGRFASFEAQGALANIAVDKANLEI
MTKRSNYTPITNVPPEVTVLTNSPVELREPNVLICFIDKFTPPVVNVTWLRNGKPVTTGVSETVFLPREDHLFR
KFHYLPFLPSTEDVYDCRVEHWGLDEPLLKHWEFDAPSPLPETTENEFGGGSGGSTTAPSAQLEKELQALEKEN
AQLEWELQALEKELAQ
DR52b beta BLZ-BSP
(SEQ ID NO: 80)
AGATCTCGACCACGTTTCTTGGAGCTGCTTAAGTCTGAGTGTCATTTCTTCAATGGGACGGAGCGGGTGCGGTT
CCTGGAGAGACACTTCCATAACCAGGAGGAGTACGCGCGCTTCGACAGCGACGTGGGGGAGTACCGGGCGGTGA
GGGAGCTGGGGCGGCCTGATGCCGAGTACTGGAACAGCCAGAAGGACCTCCTGGAGCAGAAGCGGGGCCAGGTG
GACAATTACTGCAGACACAACTACGGGGTTGGTGAGAGCTTCACAGTGCAGCGGCGAGTCCATCCTCAGGTGAC
TGTGTATCCTGCAAAGACCCAGCCCCTGCAGCACCACAACCTCCTGGTCTGCTCTGTGAGTGGTTTCTATCCAG
GCAGCATTGAAGTCAGGTGGTTCCGGAACGGCCAGGAAGAGAAGGCTGGGGTGGTGTCCACGGGCCTGATCCAG
AATGGAGACTGGACCTTCCAGACCCTGGTGATGCTAGAAACAGTTCCTCGGAGTGGAGAGGTTTACACCTGCCA
AGTGGAGCACCCAAGCGTAACGAGCCCTCTCACAGTGGAATGGAGTGCACGGTCTGAATCTGCACAGAGCAAGG
AATTCGGTGGTGGATCAGGAGGTTCAACTACAGCTCCATCAGCTCAGTTGAAAAAGAAATTGCAAGCACTGAAG
AAAAAGAACGCTCAGCTGAAGTGGAAACTTCAAGCCCTCAAGAAGAAACTCGCCCAGGGAGGCAGTGGTGGCGG
TCTGAACGACATCTTCGAGGCTCAGAAAATCGAATGGCACGAATGA
DR52b beta BLZ-BSP
(SEQ ID NO: 81)
RSRPRFLELLKSECHFFNGTERVRFLERHFHNQEEYARFDSDVGEYRAVRELGRPDAEYWNSQKDLLEQKRGQV
DNYCRHNYGVGESFTVQRRVHPQVTVYPAKTQPLQHHNLLVCSVSGFYPGSIEVRWFRNGQEEKAGVVSTGLIQ
NGDWTFQTLVMLETVPRSGEVYTCQVEHPSVTSPLTVEWSARSESAQSKEFGGGSGGSTTAPSAQLKKKLQALK
KKNAQLKWKLQALKKKLAQGGSGGGLNDIFEAQKIEWHE

Expression of Soluble Recombinant DR52b Monomers.

A million D.Mel-2 cells in 500 μl of Sf900 II SFM medium in 24-well plates were transfected with 2 μg of pMT chain DNA (1 μg each chain) and 0.2 μg pBS-PURO (Karjalainen lab) mixed with 20 μl of Cellfectin (Invitrogen) in 200 μl of Sf900 II SFM (Invitrogen). After 48 hours, puromycin was added (10 μg/ml) and cultured for 2-3 weeks to produce a stably transfected cell line.

Cells were expanded in 850 cm² (Falcon) roller bottles at ambient temperature using Sf900 II SFM medium up 5-10×10⁶ cells/ml. Protein secretion was induced with 1 mM CuSO4 (Sigma) for 5 days. The supernatants were harvested, filtered through 0.22 micron filters, supplemented with 0.05% sodium azide and 0.1% iodoacetamide.

Affinity Purification.

Empty DR52b molecules were purified by affinity chromatography on a L243 Sepharose column (L243 mouse anti-pan HLA-DR IgG2a monoclonal antibody) using for elution 50 mM glycine pH 11.5 and immediate neutralization with 2 M Tris-HCl pH 6.9. After exchange in PBS (pH 7.4), isolated DR52b was concentrated to 1-2 mg/ml.

Peptide Loading.

Peptide loading was performed in PBS, pH 7.4 or 50 mM sodium citrate, 100 mM sodium chloride, pH 5.2, depending on the peptide's solubility and features. Empty DR52b (0.1 mg/me and a peptide of interest were incubated with N-octyl-glucoside (0.2% final), EDTA (5 mM) and complete protease inhibitor tablets (Roche) according to manufacturer (1 tablet to 10.5 ml). Peptide loading was performed at 0.37° C. for at least 24 hours.

After loading, the solution containing the complexes was concentrated to 6-7 ml and buffer exchanged in BirA buffer (50 mM bicine pH 8.3, 10 mM MgOAc) and biotinylated with the BirA enzyme (Avidity) overnight at room temperature with agitation according to manufacturers' instructions.

The biotinylated complexes were exchanged into PBS (pH 7.4), concentrated to about 0.5 ml and subjected to gel filtration chromatography using a Superdex 5200 column. Fractions containing the monomer were pooled, concentrated to 1-2 mg/ml and flash frozen in liquid nitrogen (aliquots of 25-50 μg Aliquots are stored at −80° C. until use.

Biotinylation was measured by densitometry of SDS-PAGE-based avidin shift assay. Tetramer were prepared by calculating the amount of streptavidin-phycoerythrin (SA-PE) (Caltag) that has to be added to biotinylated monomers in molar ratio 1:4. The volume of SA-PE was divided in 10 aliquots and added in 5 min intervals on ice accompanied under vigorous mixing. The final concentration of the reagents was calculated as follows: (mass of biotinylated monomers+mass of SA-PE)/sum of monomer and SA-PE volumes.

### Discussion

Here, we have reported the identification of an ESO-derived DR52b-restricted epitope recognized by CD4⁺ T cells induced by vaccination with a rESO vaccine administered with the immunological adjuvants Montanide ISA-51 and CpG ODN 7909, a formulation that predominantly elicits T_{H}1 responses. Previous studies from us and others have identified ESO₁₁₉₋₁₄₃ as an immunodominant region, recognized by CD4⁺ T cells from virtually all patients with spontaneous or vaccine-induced immunity to ESO (3, 5-7, 9, 15). Several overlapping epitopes contained within the ESO₁₁₉₋₁₄₃ region and restricted by multiple HLA-DR alleles have been identified (3, 4) (summarized in Cancer Immunity Peptide Database: www.cancerimmunity.org). Surprisingly, the DR52b-restricted epitope identified in this study has not been reported thus far. Our group, however, has previously reported the identification of two other DR52b-restricted epitopes from the tumor antigens SSX-4 and Melan-A (16, 17).

At variance with the β chain of the mouse I-E molecule (homolog to human HLA-DR), encoded by a single gene, the β chain of human HLA-DR is encoded by multiple genes. In addition to the DRB1 gene, encoding the prevalent chain of the DR isotype, additional genes code for other β chains, namely DRB3 (DR52), DRB4 (DR53) and DRB5 (DR51). They are less polymorphic than DRB1 and generally expressed at lower levels, but code for DR molecules that are fully functional with respect to antigen presentation (18, 19). These genes have strong linkage disequilibrium with defined DRB1 alleles. In particular, DR52 is very frequently expressed in the population, as DRB3 alleles are associated through linkage disequilibrium to some of the most common DRB1 alleles (20). Lower expression and linkage disequilibrium with DRB1 alleles may account for the fact that T cell epitopes restricted by these alternate DR molecules have been described less frequently than those restricted by DRB1 encoded molecules, or have been reported as restricted by the associated DRB1 allele.

In general, alternate DR molecules have been less well investigated as compared to those encoded by DRB1. However, expression of several DRB3-encoded molecules has been recently reported to associate with different autoimmune diseases, which has resulted in an increased interest in investigating their structure and peptide binding specificity. Expression of DRB3*0202 (DR52b), one of the main DRB3 alleles, has been associated with Grave's disease, multiple sclerosis and essential hypertension caused by infection with Chlamydia pneumoniae (21-23).

Using truncated overlapping peptides, we defined the minimal optimal sequence recognized by ESO DR52b-restricted CD4⁺ T cells as the 15-mer 123-137. Within this sequence, a screening of the entire ESO sequence, using the MHC class II peptide binding prediction algorithm RankPep (imed.med.ucm.es/Tools/rankpep.html), identified a sequence with high predicted binding capacity to DR52b, corresponding to peptide 127-135 (TVSGNILTI), SEQ ID NO: 59. Although the crystal structure of DR52b has not been yet resolved, some structural consideration on the potential contribution of single amino acids in the identified peptide to binding can be drawn based on previous analyses of natural peptides isolated from DR52 molecules and on the recently reported structure of the highly homologous DR52c molecule bound to a self-peptide derived from the Tu elongation factor (24, 25). The most salient feature of the identified peptide is the amino acid N located in the central part of the sequence. Together with DR52c, and at variance with most other DR molecules, DR52b has a Q at position P74, that together with other residues in the P4 pocket, limit the amino acids binding at this position to N or D, whereas the P1 and P6 pockets are expected to be rather permissive and can accommodate many different residues.

DR52b is expressed by about half of Caucasians, which is similar to the frequency of expression of the most investigated human MHC class I molecule: HLA-A*0201. The frequent expression of HLA-A*0201 has allowed extensive analysis, including assessment of ex-vivo frequency and phenotype of tumor antigen-specific HLA-A*0201-restricted CTL (including Melan-A and ESO-specific) using HLA-A*0201/peptide fluorescent tetramers (2, 26, 27). The identification of the ESO DR52b-restricted epitope may allow the development of a similar approach using MHC class II/peptide tetramers to assess CD4⁺ T cell responses to ESO. This is particularly relevant taking into account the immunodominant character of ESO-specific DR52b-restricted CD4⁺ T cell responses, following vaccination. Indeed, by assessing the quantitative contribution of DR52b-restricted responses to the overall response to the immunodominant 119-143 region, following vaccination, we demonstrate that, although variable among different individuals, these represented in average about 50% of total specific CD4⁺ T cells. The prevalence of DR52b-restricted CD4⁺ T cells in patients with spontaneous responses to ESO, however, might be different and remains to be determined.

ESO-specific DR52b-restricted CD4⁺ T cell clones isolated in this study efficiently recognized the natural exogenous ESO antigen after processing and presentation by APC but failed to recognize endogenously expressed ESO. We have previously obtained similar results with CD4⁺ T cell clones specific for another CTA, SSX-4 (16) whereas we have observed recognition of both exogenous and endogenously expressed antigen using Melan-A specific CD4⁺ T cells (17). The ability of CD4⁺ T cells to recognize endogenously expressed tumor antigens may be epitope dependent (28, 29) and can significantly vary for different tumor antigens, depending on their intracellular localization. At variance with CTAs, melanocyte differentiation antigens such as Melan-A have a natural access to the endogenous MHC class II processing and presentation pathway, as they are localized in melanosomes, or, in their absence, in lysosomes (30). Generation of ESO-specific CD4⁺ T cells prevalently recognizing exogenously processed antigen is expected following vaccination with recombinant ESO protein. However, as direct recognition of tumor cells is most likely not the dominant mechanism through which tumor antigen-specific CD4⁺ T cells contribute to tumor rejection (31-33), lack of recognition of endogenous ESO antigen by CD4⁺ T cells does not necessarily imply a decreased importance of this epitope in the effector phase of anti-tumor immune response to ESO expressing tumors. To assess this point, it would be of interest to assess the presence of specific DR52b-restricted CD4⁺ T cells among tumor infiltrating lymphocytes from patients with spontaneous immunity to ESO.

By assessing the TCR of ESO₁₁₉₋₁₄₃-specific DR52b-restricted CD4⁺ T clones from different individuals, we could demonstrate conserved TCR usage, with frequent usage of BV2 often in association with BJ 2.1. The CDR3 region of the different clonotypes identified, however, was variable, both in terms of length and amino acid composition, which could indicate a certain degree of heterogeneity in the fine specificity and/or avidity of antigen recognition among different clones. We have previously reported conserved but distinct TCR usage for ESO-specific HLA-A*0201-restricted CTL that occur naturally or are induced through peptide vaccination (34), which was associated with their ability to recognize or not the naturally processed antigen. This is, however, in our knowledge, the first study assessing TCR usage by ESO-specific CD4⁺ T cells. It will therefore be of interest to compare the TCR usage of ESO₁₁₉₋₁₄₃-specific DR52b-restricted CD4⁺ T cells elicited by vaccination with that of CD4⁺ T cells naturally occurring in patients with spontaneous immunity to ESO. Interestingly, Kudela P. et al. have recently reported the existence of promiscuous clonal CD4⁺ T cells able to recognize peptide ESO₁₁₉₋₁₄₃ in the context of several distinct MHC class II molecules (35). Although the CD4⁺ T cell clones isolated in the present study did not display promiscuous antigen recognition (data not shown), it will be clearly of great interest, in future studies, to compare their TCR usage to those of monogamous or promiscuous CD4⁺ T clones recognizing other epitopes in the 119-143 region.

### REFERENCES

1. Scanlan M J, Gure A O, Jungbluth A A, Old L J, and Chen Y T. Cancer/testis antigens: an expanding family of targets for cancer immunotherapy. Immunol Rev 2002; 188:22-32.
2. Valmori D, Dutoit V, Lienard D, et al. Naturally occurring HLA-A2 restricted CD8+ T cell response to the cancer testis antigen NY-ESO-1 in melanoma patients. Cancer Res 2000; 60:4499-506.
3. Jager E, Jager D, Karbach J, et al. Identification of NY-ESO-1 epitopes presented by human histocompatibility antigen (HLA)-DRB4*0101-0103 and recognized by CD4(+) T lymphocytes of patients with NY-ESO-1-expressing melanoma. J Exp Med 2000; 191:625-30.
4. Zarour H M, Storkus W J, Brusic V, Williams E, and Kirkwood J M. NY-ESO-1 encodes DRB1*0401-restricted epitopes recognized by melanoma-reactive CD4+ T cells. Cancer Res 2000; 60:4946-52.
5. Davis I D, Chen W, Jackson H, et al. Recombinant NY-ESO-1 protein with ISCOMATRIX adjuvant induces broad integrated antibody and CD4(+) and CD8(+) T cell responses in humans. Proc Natl Acad Sci USA 2004; 101:10697-702.
6. Mandic M, Castelli F, Janjic B, et al. One NY-ESO-1-derived epitope that promiscuously binds to multiple HLA-DR and HLA-DP4 molecules and stimulates autologous CD4+ T cells from patients with NY-ESO-1-expressing melanoma. J Immunol 2005; 174:1751-9.
7. Ayyoub M, Souleimanian N E, Godefroy E, et al. A phenotype based approach for the immune monitoring of NY-ESO-1 specific CD4+ T cell responses in cancer patients. Clin Immunol 2006; 118:188-94.
8. Zeng G, Wang X, Robbins P F, Rosenberg S A, and Wang R F. CD4(+) T cell recognition of MHC class II-restricted epitopes from NY-ESO-1 presented by a prevalent HLA DP4 allele: association with NY-ESO-1 antibody production. Proc Natl Acad Sci USA 2001; 98:3964-9.
9. Valmori D, Souleimanian N E, Tosello V, et al. Vaccination with NY-ESO-1 protein and CpG in Montanide induces integrated antibody/Th1 responses and CD8 T cells through cross-priming. Proc Natl Acad Sci USA 2007; 104:8947-52.
10. Valmori D. Dutoit V, Lienard D, et al. Tetramer-guided analysis of TCR (3-chain usage reveals a large repertoire of Melan-A-specific CD8+ T cells in melanoma patients. J. Immunol. 2000; 165:533-8.
11. Genevee C, Diu A, Nierat J, et al. An experimentally validated panel of subfamily-specific oligonucleotide primers (V alpha 1-w29N beta 1-w24) for the study of human T cell receptor variable V gene segment usage by polymerase chain reaction. Eur J Immunol 1992; 22:1261-9.
12. Arden B, Clark S P, Kabelitz D, and Mak T W. Human T-cell receptor variable gene segment families. Immunogenetics 1995; 42:455-500.
13. Ayyoub M, Hesdorffer C S, Montes M, et al. An immunodominant SSX-2-derived epitope recognized by CD4+ T cells in association with HLA-DR. J Clin Invest 2004; 113:1225-33.
14. Rimoldi D, Rubio-Godoy V, Dutoit V, et al. Efficient simultaneous presentation of NY-ESO-1/LAGE-1 primary and nonprimary open reading frame-derived CTL epitopes in melanoma. J Immunol 2000; 165:7253-61.
15. Zarour H M, Maillere B, Brusic V, et al. NY-ESO-1 119-143 is a promiscuous major histocompatibility complex class II T-helper epitope recognized by Th1- and Th2-type tumor-reactive CD4+ T cells. Cancer Res 2002; 62:213-8.
16. Valmori D, Qian F, Ayyoub M, et al. Expression of synovial sarcoma X (SSX) antigens in epithelial ovarian cancer and identification of SSX-4 epitopes recognized by CD4+ T cells. Clin Cancer Res 2006; 12:398-404.
17. Godefroy E, Scotto L, Souleimanian N E, et al. Identification of two Melan-A CD4+ T cell epitopes presented by frequently expressed MHC class II alleles. Clin Immunol 2006; 121:54-62.
18. Texier C, Pouvelle-Moratille S, Busson M, Charron D, Menez A, and Maillere B. Complementarity and redundancy of the binding specificity of HLA-DRB1, -DRB3, -DRB4 and -DRB5 molecules. Eur J Immunol 2001; 31:1837-46.
19. Cotner T, Charbonneau H, Mellins E, and Pious D. mRNA abundance, rather than differences in subunit assembly, determine differential expression of HLA-DR beta 1 and -DR beta 3 molecules. J Biol Chem 1989; 264:11107-11.
20. Sengar D P, Goldstein R, Toye B, and Hampton N. Comprehensive typing of DR52 (DRB3)-associated DRB1 and DRB3 alleles by PCR-RFLP. Tissue Antigens 1994; 43:286-94.
21. Chen Q Y, Huang W, She J X, Baxter F, Volpe R, and Maclaren N K. HLA-DRB1*08, DRB1*03/DRB3*0101, and DRB3*0202 are susceptibility genes for Graves' disease in North American Caucasians, whereas DRB1*07 is protective. J Clin Endocrinol Metab 1999; 84:3182-6.
22. Oh H H, Kwon S H, Kim C W, et al. Molecular analysis of HLA class II-associated susceptibility to neuroinflammatory diseases in Korean children. J Korean Med Sci 2004; 19:426-30.
23. Zabay J M, Marco J, Soler J, et al. Association of HLA-DRB3*0202 and serum IgG antibodies to Chlamydia pneumoniae with essential hypertension in a highly homogeneous population from Majorca (Balearic Islands, Spain). J Hum Hypertens 2005; 19:615-22.
24. Verreck F A, van de Poel A, Drijfhout J W, Amons R, Coligan J E, and Konig F. Natural peptides isolated from Gly86/Val86-containing variants of HLA-DR1, -DR11, -DR13, and -DR52. Immunogenetics 1996; 43:392-7.
25. Dai S, Crawford F, Marrack P, and Kappler J W. The structure of HLA-DR52c: comparison to other HLA-DRB3 alleles. Proc Natl Acad Sci USA 2008; 105:11893-7.
26. Dutoit V, Taub R N, Papadopoulos K P, et al. Multiepitope CD8(+) T cell response to a NY-ESO-1 peptide vaccine results in imprecise tumor targeting. J Clin Invest 2002; 110:1813-22.
27. Valmori D, Dutoit V, Schnuriger V, et al. Vaccination with a Melan-A peptide selects an oligoclonal T cell population with increased functional avidity and tumor reactivity. J Immunol 2002; 168:4231-40.
28. Chaux P, Vantomme V, Stroobant V, et al. Identification of MAGE-3 epitopes presented by HLA-DR molecules to CD4(+) T lymphocytes. J Exp Med 1999; 189:767-78.
29. Manici S, Sturniolo T, Imro M A, et al. Melanoma cells present a MAGE-3 epitope to CD4(+) cytotoxic T cells in association with histocompatibility leukocyte antigen DR11. J Exp Med 1999; 189:871-6.
30. Levy F, Muehlethaler K, Salvi S, et al. Ubiquitylation of a melanosomal protein by HECT-E3 ligases serves as sorting signal for lysosomal degradation. Mol Biol Cell 2005; 16:1777-87.
31. Hung K, Hayashi R, Lafond-Walker A, Lowenstein C, Pardoll D, and Levitsky H. The central role of CD4(+) T cells in the antitumor immune response. J Exp Med 1998; 188:2357-68.
32. Qin Z and Blankenstein T. CD4+ T cell—mediated tumor rejection involves inhibition of angiogenesis that is dependent on IFN gamma receptor expression by nonhematopoietic cells. Immunity 2000; 12:677-86.
33. Mumberg D, Monach P A, Wanderling S, et al. CD4(+) T cells eliminate MHC class II-negative cancer cells in vivo by indirect effects of IFN-γ. Proc Natl Acad Sci USA 1999; 96:8633-8.
34. Le Gal F A, Ayyoub M, Dutoit V, et al. Distinct structural TCR repertoires in naturally occurring versus vaccine-induced CD8+ T-cell responses to the tumor-specific antigen NY-ESO-1. J Immunother 2005; 28:252-7.
35. Kudela P, Janjic B, Fourcade J, et al. Cross-reactive CD4+ T cells against one immunodominant tumor-derived epitope in melanoma patients. J Immunol 2007; 179:7932-40.

The entire contents of all of the references (including literature references, issued patents, published patent applications, and co-pending patent applications) cited throughout this application are hereby expressly incorporated by reference for the purposes or subject matter referenced herein.

### Example 2

Generation of HLA-DR52b/ESO Peptide Tetramers.

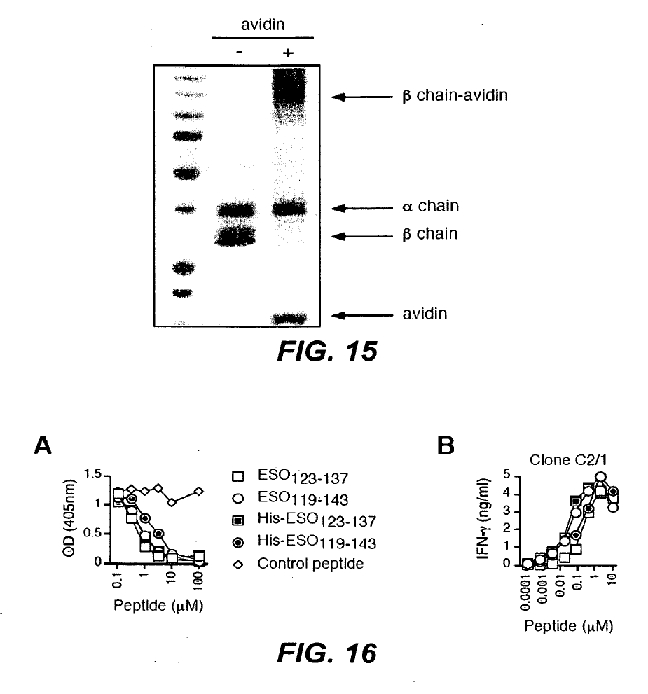

The extracellular domain of the DR52b beta chain was amplified from total cDNA (Qiagen AG) obtained from the DR52b⁺ EBV-immortalized B cell line EBV-B14 using ctttagatctcgaccacgtttcttggagc (SEQ ID NO: 83) as the 5′ primer and ctttgaattccttgctctgtgeagattcag (SEQ ID NO: 84) as the 3′ primer and cloned in the pMT A vector (Invitrogen AG)-derived cassette containing sequences coding for a C-terminal basic leucine zipper followed by an AviTag (Avidity) as previously described (32). D. mel-2 cells (Invitrogen) were transfected with constructs encoding the DR alpha and DR52b beta chains together with the pBS-PURO (gift from Dr. K. Karjalainen, Nanyang Technological University, School of Biological Sciences, Singapore) in a 10:1 ratio with Cellfectin (Invitrogen). After selection in Sf900 II SFM medium (Invitrogen) containing 10 μg/ml puromycin (Sigma Aldrich), cells were cloned by limiting dilution and clones with the highest expression of soluble DR52b protein were used for large scale production in roller bottles. Protein expression was induced by addition of 1 mM CuSO4 for 3 to 5 days and soluble DR52b was purified from supernatants with anti-DR (clone L243) affinity chromatography. Yields of >2 mg/L were routinely obtained. The DR52b eluate was immediately brought to optimal peptide loading pH of 6.0 with 100 mM citric acid, loaded at a peptide to protein molar ratio of 50:1, at 28° C. for 24 hrs in the presence of protease inhibitor cocktail (Roche) and 0.2% octyl 13-D-glucopyranoside (Sigma) and then biotinylated using the BirA enzyme (Avidity). When DR52b molecules were loaded with untagged ESO peptides, pMHC complexes were directly purified by gel filtration in PBS pH 7.4, 100 mM NaCl on a Superdex S200 column (GE Healthcare Life Sciences) and the fractions corresponding to the monomeric pMHC complexes were pooled and concentrated. Alternatively, ESO peptides were extended at the N-terminus by a sequence containing 6 His residues and a linker (Ser-Gly-Ser-Gly). DR52b/His-tag-ESO peptide complexes were purified using the H isTrap HP 1 ml column (GE Healthcare Life Sciences) prior to purification by gel filtration (FIG. 14). Briefly, the sample was applied in PBS pH 7.4, 100 mM NaCl and 10 mM imidazole and after washing eluted with 200 mM imidazole in the same buffer. Finally, biotinylation and purity, as assessed by SDS-PAGE in an avidin shift assay, were both >90% (not shown and FIG. 15). Biotinyated DR52b/peptide complexes were multimerized by mixing with small aliquots of streptavidin-PE (Invitrogen) up to the calculated 4:1 stoichiometrical amount.

Patients Samples, Cells, Tissue Culture, Tetramer Staining and Flow Cytometry Analysis and Sorting.

Peripheral blood samples were collected from cancer patients enrolled in a clinical trial of vaccination with recombinant ESO protein, Montanide ISA 51 and CpG 7909 (8) upon informed consent and approval by the Institutional Review Boards. Peripheral blood samples from healthy donors were obtained from the Etablissement Francais du Sang Pays de la Loire (Nantes, France). MHC class II alleles were determined by high resolution molecular typing (9). ESO₁₁₉₋₁₄₃-specific DR52b-restricted CD4⁺ T cell clonal populations were obtained from post-vaccine samples as previously described (9). For assessment of specific CD4⁺ T cell responses following in vitro stimulation, CD4⁺ cells were enriched from PBMC by magnetic cell sorting (Miltenyi Biotec Inc.), stimulated with irradiated autologous APC in the presence of a pool of overlapping long peptides spanning the ESO sequence, rhIL-2 and rhIL-7 as previously described (9) and maintained in culture during 10-15 days prior to tetramer staining. Peptide stimulated cultures and clonal populations were incubated with tetramers at a final concentration of 3 μg/ml for 1 hr at 37° C., unless otherwise indicated, in complete IMDM medium, washed and then stained with CD4 (BD Biosciences) or TCR Vβ (Beckman Coulter) specific mAb in PBS, 5% FCS for 15 minutes at 4° C. and analyzed by flow cytometry (FACSAria, BD Biosciences). In order to generate specific polyclonal T cell populations, tetramer⁺ cells within peptide-stimulated cultures were sorted by flow cytometry (FACSAria, BD Biosciences) and expanded by stimulation with PHA and irradiated allogeneic PBMC in the presence of rhIL-2 (33). For ex vivo enumeration and phenotyping of specific cells, CD4⁺ cells enriched from PBMC were rested overnight, incubated with tetramers (3 μg/ml) for 2 hours at 37° C. and then stained with CD45RA, CCR7, CD25, CD27, CD28 and CD127 specific mAb, as indicated, and analyzed by flow cytometry.

Antigen Recognition Assays.

DR52b⁺ ESO-specific CD4⁺ T cell clones or polyclonal cultures were stimulated in the absence or presence of ESO peptides (2 μM) or PMA (100 ng/ml) and ionomycin (1 μg/ml), as indicated, and cytokine production was assessed in a standard 4 hrs intracellular cytokine staining assay using mAb specific for IFN-γ, TNF-α, IL-2, IL-4, IL-10 (BD Biosciences) and IL-17 (eBiosciences), as previously described (9, 34). In other experiments, specific polyclonal cultures were incubated for 24 hrs with either DR52b⁺ EBV-B cells and serial dilutions of ESO peptides or monocyte derived dendritic cells pre-incubated overnight with serial dilutions of rESO, and IFN-γ was measured by ELISA (Invitrogen) in 24 hrs culture supernatants, as previously described (8, 9).

Generation of Molecularly Defined MHC Class II Tetramers Using His-tag-Peptides and their Validation.

We initially attempted to generate DR52b tetramers incorporating peptide ESO₁₂₃₋₁₃₇ using a strategy previously described by Kwok et al. (10). The tetramers synthesized according to this procedure, however, failed to significantly stain ESO-specific DR52b-restricted CD4⁺ T cell clones (FIG. 13, A and B). We reasoned that this failure might be due to a suboptimal formation of DR52b/ESO complexes, resulting in the presence of low proportions of folded monomers in the tetramer preparation. To overcome this limitation, we synthesized an ESO peptide containing an N-terminal His-tag added via a short linker. After loading DR52b molecules with the His-tag peptide, monomers were purified by affinity chromatography on Ni²⁺-NTA columns, followed by gel filtration chromatography (FIG. 14). The purity of the isolated biotinylated monomers was assessed in a shift assay with avidin (FIG. 15). Tetramerization was carried on using phycoerythrin (PE)-labeled streptavidin. The tetramers prepared using this novel procedure efficiently stained ESO-specific DR52b-restricted CD4⁺ clonal T cells, at low concentrations, similar to those generally used for MHC class. I/peptide tetramers (11, 12), with low background on control populations (FIG. 6, A and B). To further assess the staining obtained with molecularly defined tetramers, we tested the influence of temperature and incubation time. No significant staining was detectable upon incubation at 4° C., even after prolonged incubation (FIG. 6C). We observed low but significant staining at 23° C., particularly after long incubation. Staining at 37° C., however, was much more efficient and displayed a more rapid kinetic. To assess the persistence of tetramer staining, we incubated specific clones with tetramers during 1 hr at 37° C., removed the excess tetramers by washing and incubated the cells at various temperatures for different times. No decrease in the staining intensity was detected upon incubation at 4° C. or 23° C., up to 24 hrs (FIG. 6D). Even at 37° C., the staining was maintained up to 4 hrs and gradually decreased afterwards, remaining detectable at 24 hrs. Together, these results show that molecularly defined DR52b/ESO tetramers avidly and stably bind specific CD4⁺ T cells with negligible background staining on non-specific CD4⁺ T cells.

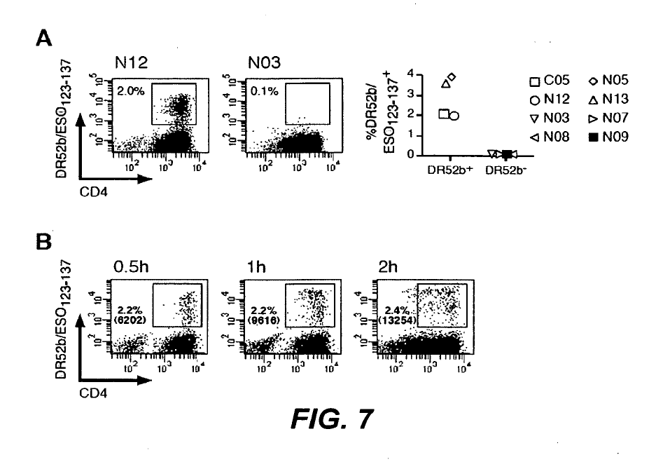

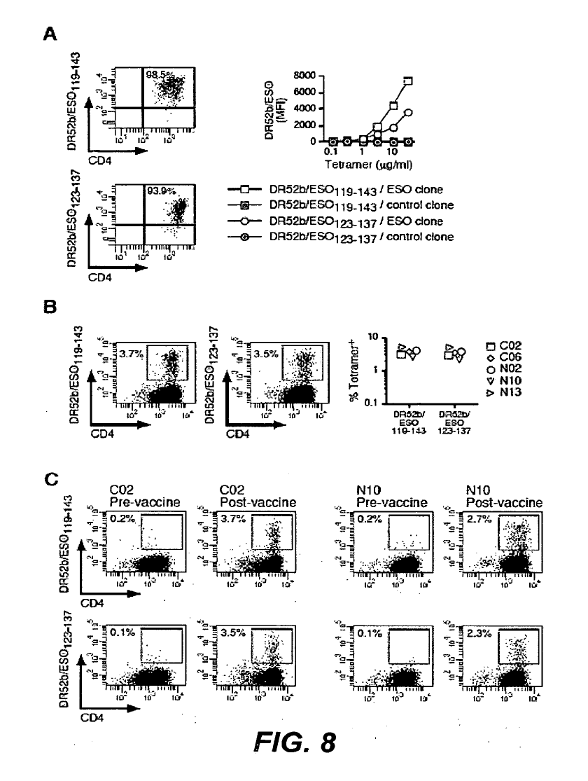

To assess the capacity of the tetramers to identify specific CD4⁺ T cells within polyclonal populations, we stained peptide-stimulated cultures from DR52b⁺ and DR52b cancer patients immunized with the rESO vaccine (8). After 1 hr incubation at 37° C., DR52b/ESO tetramers stained a significant proportion of CD4⁺ T cells in the cultures from DR52b⁺ but not from DR52b⁻ patients (FIG. 7A). On selected cultures, we compared the staining obtained after incubation for different time periods. Similar proportions of tetramer⁺ cells were detected after incubation for different times, with the mean fluorescence intensity of the tetramer⁺ populations being higher after longer incubation periods (FIG. 7B).

Whereas peptide ESO₁₂₃₋₁₃₇ was the minimal peptide optimally recognized by clonal CD4⁺ T cells, the ESO peptide originally used to identify the DR52b-restricted epitope was significantly longer, corresponding to the 25mer ESO₁₁₉₋₁₄₃ (9). We therefore synthesized DR52b/ESO₁₁₉₋₁₄₃ tetramers and assessed them on specific and control clonal populations. Similar to ESO₁₂₃₋₁₃₇ tetramers, tetramers prepared with untagged ESCO₁₁₉₋₁₄₃ failed to stain ESO-specific clones (not shown). In contrast, DR52b/ESO₁₁₉₋₁₄₃ tetramers prepared using a His-tag ESO₁₁₉₋₁₄₃ peptide and purified monomeric complexes stained specific clonal populations with a slightly increased efficiency as compared to DR52b/ESO₁₂₃₋₁₃₇ tetramers and displayed similar low background on control clones (FIG. 8A). Both DR52b/ESO₁₂₃₋₁₃₇ and DR52b/ESO₁₁₉₋₁₄₃ tetramers identified similar proportions of specific CD4⁺ T cells in peptide-stimulated cultures from post-vaccination samples and failed to detect specific cells in peptide-stimulated cultures from samples obtained prior to vaccination (FIG. 8, B and C).

Because our new approach involves the addition of a His-tag to the ESO peptides, it was important to address the effect of this modification on peptide binding to class II molecules and recognition by specific CD4⁺ T cells. With this aim we assessed the relative efficiency of untagged and His-tagged ESO peptides to bind to DR52b using a previously described competition assay (13). As shown in FIG. 16A, addition of the His-tag, surprisingly, did not significantly modify peptide binding to DR52b for both ESO₁₂₃₋₁₃₇ and ESO₁₁₉₋₁₄₃. In addition, whereas the His-tagged ESO₁₂₃₋₁₃₇ was recognized by specific CD4⁺ T cells with moderately improved efficiency, untagged and His-tagged ESO₁₁₉₋₁₄₃ peptides were recognized with similar efficiency (FIG. 16B). Together these data demonstrate that the success of the new approach in generating efficient tetramers is not due to the effect of the His-tag itself on peptide binding or T cell recognition, but to its use to purify folded DR52b/ESO peptide complexes.

Molecularly Defined DR52b/ESO Tetramers Allow Direct Ex Vivo Enumeration and Phenotyping of CD4⁺ T Cells Induced by the rESO Vaccine.

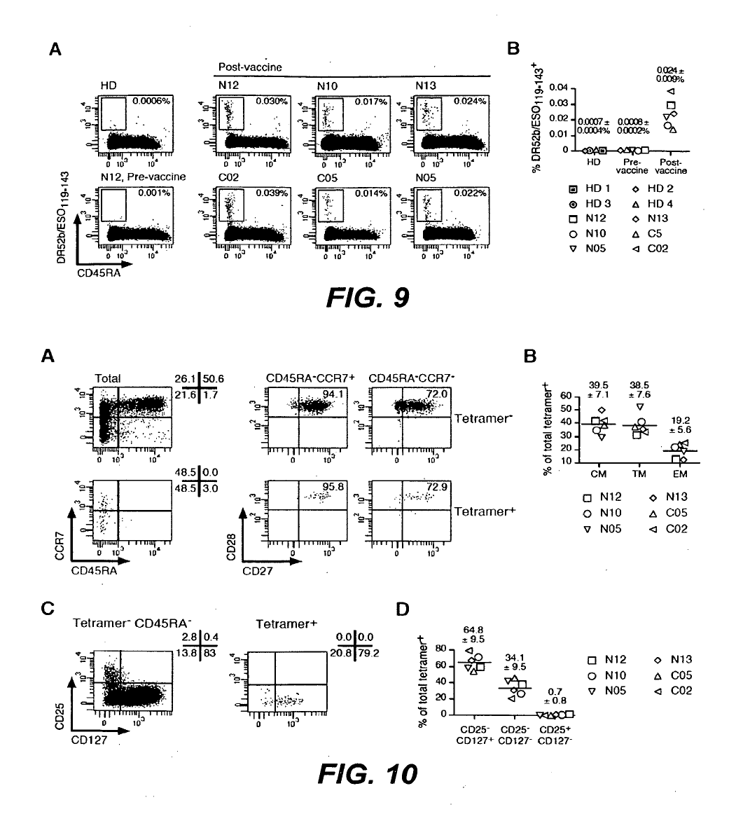

The high efficiency and specificity of staining obtained with the molecularly defined DR52b/ESO tetramers prompted us to assess their capacity to detect vaccine-induced CD4⁺ T cells ex vivo. With this aim, we isolated CD4⁺ T cells from PBMC of DR52b⁺ healthy donors and patients using magnetic cell sorting; and stained them with the tetramers during 2 hrs at 37° C. in combination with CD45RA-specific antibodies. As shown in FIG. 9, the frequency of DR52b/ESO tetramer⁺ cells among CD4⁺CD45RA⁻ T cells from healthy donors was below 1:100000. We obtained similar results when assessing CD4⁺ T cells from patients prior to vaccination. In contrast, in post-vaccine samples, DR52b/ESO tetramer⁺ cells were clearly detectable among CD4⁺CD45RA⁻ T cells at a frequency ranging between 1:2500 and 1:7000 (average 1:5000). The quality of memory CD4⁺ T cell responses elicited by pathogens or vaccines has been correlated with their phenotype. Specifically, it has been inferred that a protective memory response should include not only effector cells (CCR7⁺) but also significant proportions of “reservoir” memory cells, including central memory (CCR7⁺) and transitional memory (CCR7⁻CD27⁺) populations (14, 15). To more extensively characterize vaccine-induced CD4⁺ T cells, we co-stained them with tetramers and antibodies directed against markers that distinguish distinct differentiation stages of memory cells. This analysis revealed that vaccine-induced DR52b/ESO tetramer⁺ populations included significant proportions of CCR7⁺ central memory cells (FIG. 10, A and B). In addition, among tetramer⁺ CCR7⁻ cells, the majority were CD27⁺ transitional memory T cells. Another important criterion to select candidate anti-cancer vaccines is their ability to elicit helper CD4⁺ T cell responses, but not suppressive CD25⁺CD127⁻ Treg (16, 17). To address this point, we co-stained post-vaccine CD4⁺ T cells with DR52b/ESO tetramers in combination to antibodies to CD45RA, CD25 and CD127. As shown in (FIG. 10, C and D), whereas CD25⁺CD127⁻ Treg populations were clearly detected among CD4⁺ T cells of vaccinated patients, the large majority of vaccine-induced tetramer⁺ cells were CD25⁻CD127⁺. These results clearly demonstrate that the rESO/Montanide/CpG vaccine mainly induces central and transitional memory CD4⁺ T cells and does not induce ESO-specific Treg.

MHC Class II Tetramer-Guided Isolation and Functional Characterization of ESO-Specific CD4⁺ T Cells.

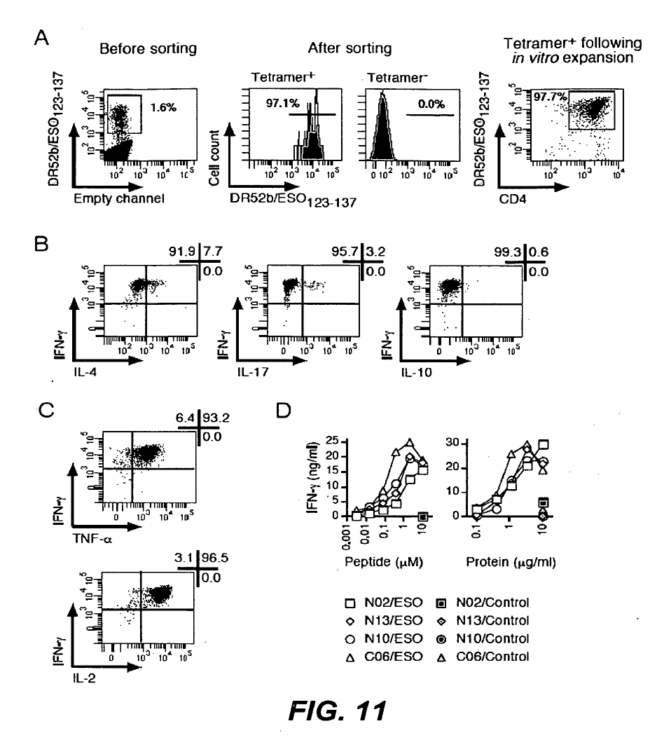

To assess vaccine-induced CD4⁺ T cells functionally, we isolated them by tetramer-guided flow cytometry cell sorting and expanded them in vitro (FIG. 11A). The resulting populations contained >90% tetramer⁺ cells. To address the type of CD4⁺ T cell response induced by the vaccine, we used antibodies directed against different cytokines that characterize different T_{H} subsets. Vaccine-induced tetramer⁺ cells displayed a clear T_{H}1 profile, as they mainly produced IFN-γ and contained only minor proportions of IL-4 and IL-17 secreting cells and no detectable IL-10 secreting cells (FIG. 11B). CD4⁺ T cell populations able to produce TNF-α and IL-2 in addition to IFN-γ (called polyfunctional) are associated with enhanced cellular-mediated protection (18). As illustrated in FIG. 11C, the large majority of DR52b/ESO tetramer⁺ cells were polyfunctional as they co-secreted IFN-γ, TNF-α and IL-2. To get insight into the functional avidity of antigen recognition of the tetramer⁺ cell populations we assessed them using DR52b⁺ EBV-B as APC incubated with serial dilutions of ESO peptide. All populations specifically recognized the peptide, displaying 50% maximal recognition at a concentration comprised between 0.1 and 1 μM (FIG. 11D). Tetramer⁺ cell populations were also able to recognize rESO processed and presented by DR52b⁺ monocyte-derived dendritic cells, with 50% maximal recognition of the protein in the same range of concentrations as that of the peptide.

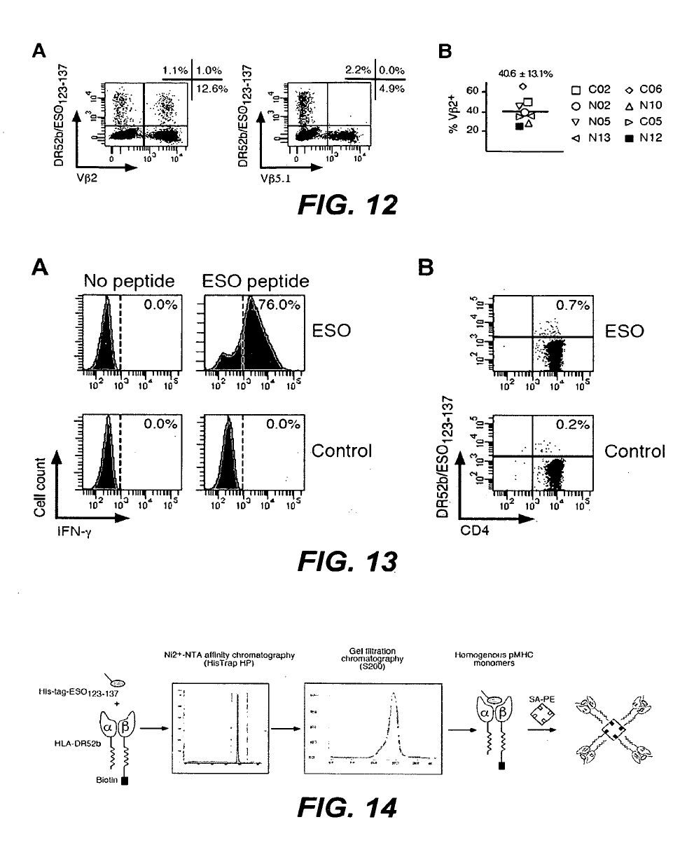

We have previously reported that more than 50% of ESO-specific DR52b-restricted CD4⁺ T cell clones isolated from vaccinated patients use Vβ2, suggesting a highly restricted TCR repertoire for T cells recognizing this epitope (9). To directly assess Vβ2 usage by tetramer⁺ cells, we co-stained the cultures with tetramers and anti-Vβ2 specific antibodies. To minimize inhibition of tetramer binding by anti-Vβ antibodies, we first incubated the cultures with tetramers during 1 hr at 37° C., washed them, and then incubated them with anti-Vβ2 antibodies. Under these conditions, we detected a proportion of tetramer⁺ Vβ2⁺ T cells in the cultures comprised between 25-65% (FIG. 12). To address if other Vβ frequently used by tetramer⁺ CD4⁺ T cells could be identified by this approach, we co-stained some of the cultures with the tetramers and a panel of anti-Vβ antibodies covering together about 50% of the TCR repertoire. This approach, however, failed to identify other relevant Vβ (not shown).

### Discussion

Because of the popularity of MHC class I/peptide tetramers, originally described in 1996 and used since in thousands of studies, attempts to generate efficient MHC class II/peptide tetramers have been pursued during the last decade, yet have met only modest success. Fundamental structural differences between MHC class I and class II molecules have required significantly different approaches for their design. For class I molecules, refolding of the single heavy chain in the presence of peptides and β₂-microglobulin yields folded stable monomeric complexes (19). Class II molecules, however, are noncovalent dimers of cc and p chains that display variable stability in solution (20). To reliably generate stable class II molecules in soluble form, the group of Kwok has constructed class II molecules incorporating leucine zipper motifs that replace the transmembrane and cytoplasmic portions of the molecules (10, 21). The advantage of this approach, with respect to others involving the synthesis of covalent single chain class II/peptide molecules (22), is that empty class II molecules can be loaded with any selected peptide, increasing tremendously the number of epitopes that can be studied. The disadvantage, however, is that, as the α and β chains complex is formed irrespective of the antigenic peptide, the proportion of folded class II/peptide complexes in the preparation can be highly variable for different peptides. Thus, whereas this approach has been successfully used in some cases, it has not been generally applicable for the study of a large variety of antigenic peptides, particularly those derived from tumor and self-antigens, that often bind class II molecules with lower affinity than those derived from pathogens (2-5).

Another problem in generating class II tetramers for generic use is the extensive polymorphism in humans, particularly in the case of the DRB1 gene encoding the prevalent β chain of the DR isotype. At variance with the β chain of the mouse I-E molecule (homolog to HLA-DR) that is encoded by a single gene, in humans several additional genes encode other β chains, namely DRB3 (DR52), DRB4 (DR53) and DRB5 (DR51). Whereas DRB1 is present in all individuals, DRB3, DRB4 and DRB5 are only present in part of them, and are in strong linkage disequilibrium with defined DRB1 alleles. These alternate DR molecules are generally expressed at lower levels as compared to those encoded by DRB1, but are fully functional with respect to antigen presentation (13, 23, 24). They are less polymorphic than DRB1-encoded molecules and represent therefore ideal candidates for the generation of generic class II tetramers (25). In particular DR52b, encoded by one of the main DRB3 alleles, is expressed by half of Caucasians. In the last years, an increasing number of studies have concentrated on alternate DR molecules, describing their structure and binding characteristics and peptide binding motifs have been defined for several of them (13, 26).

Because of the failure of our initial attempts to generate efficient DR52b/ESO tetramers by peptide loading of DR52b molecules incorporating leucine zipper motifs as previously described by Kwok et al. (10, 21), we designed a new strategy that uses His-tagged peptides, enabling the isolation of folded class II/peptide monomers by affinity purification prior to tetramer formation. Together, the data reported in this study clearly show that molecularly defined DR52b/ESO tetramers are reliable reagents for the detection, characterization and isolation of ESO-specific CD4⁺ T cells. We obtained efficient staining of clonal and polyclonal ESO-specific DR52b-restricted CD4⁺ T cell populations using concentrations of molecularly defined class II tetramers similar to those generally used for class I/peptide tetramers (1-10 μg/ml) (11, 12). Consistent with other reports (27, 28), and at variance with most class I/peptide tetramers that efficiently stain specific CD8⁺ T cells at 4° C. or at 23° C., efficient staining with class II tetramers was optimally achieved upon incubation at 37° C. The molecular basis for this difference, that might be in relation with a lower functional avidity of CD4⁺ T cells and/or with a higher need for TCR clustering, remains to be fully elucidated. Importantly, we obtained efficient staining not only using tetramers incorporating the previously defined minimal peptide required for optimal T cell recognition (15 amino acids long, ESO₁₂₃₋₁₃₇), but also using a His-tagged 25 amino acid long peptide, ESO₁₁₉₋₁₄₃, extended at both the N- and C-terminal ends of the core region. This indicates that there are no major limitations in the length of peptides that can be incorporated into DR52b molecules and implies that the use of molecularly defined DR52b tetramers incorporating long peptides from defined protein regions, possibly pre-selected on the basis of the presence of binding motifs or through functional binding assays, may be an efficient strategy allowing the rapid identification of immunodominant DR52b epitopes from a large number of antigens.

For example, in some embodiments, a set of overlapping peptides covering the entire sequence, or a specific fragment, of a protein antigen of interest are generated. In some embodiments, the peptides are tagged, for example, with a His-tag. In some embodiments, the peptides each comprise a sequence of about 20 to about 40 contiguous amino acids of the antigen. In some embodiments, the peptides each comprise a sequence of about 25 to about 30 contiguous amino acids. In some embodiments, MHC class II molecules, for example, DR52b molecules, are loaded with such peptides, for example, by contacting a MHC class II molecule with a specific tagged peptide for peptide loading in order to avoid competition for binding between the different peptides. In some embodiments, peptide-loaded MHC class II monomers are generated in this manner. In some embodiments, peptide-loaded MHC class II monomers are further assembled into peptide-loaded MHC class II multimers, for example, tetramers. Accordingly, in some embodiments, a set of peptide-loaded MHC class II monomers and/or multimers, for example, tetramers, is generated for a protein antigen of interest comprising monomers or multimers loaded with overlapping peptides covering the whole sequence, or a fragment thereof, of a protein antigen of interest. In some embodiments, different peptide-loaded MHC class II molecules or multimers from a set of such molecules covering the whole sequence, or a fragment thereof, of a protein antigen are combined. In some embodiments, such mixtures of sets of MHC class II monomers or multimers, for example, tetramers, are used to screen CD4+ T cells. In some embodiments, if reactivity of CD4+ T cells to a mixture of peptide-loaded MHC class II monomers or multimers is detected, MHC class II molecules loaded with individual peptides are tested separately to identify an epitope of the protein antigen and/or to isolate CD4+ T cells specifically binding an epitope. Such epitope mapping methods based on peptide-loaded MHC class II molecules, as provided by some aspects of this invention, depend on the expression of the antigen-specific T cell receptor and are, in contrast to conventional mapping and isolation methods, not dependent on the function of the targeted T cells (e.g., on cytokine secretion, upregulation of activation markers, etc.), which can be highly variable for different T cells.

These findings are also compatible with the fact that, in contrast to the strict length requirement of class I bound peptides (8-10 mers) that need to perfectly fit a groove that is closed at both ends, often by adopting a kinked conformation, the class II binding groove, open at both ends, can easily accommodate long peptides (15-25 mers) that bind in an extended form (29, 30). Because of the high quality of the molecularly defined tetramers, we could identify, enumerate and phenotype ex vivo ESO-specific CD4⁺ T cells induced by immunization of cancer patients with a rESO vaccine that is presently under trial for cancer immunotherapy. This allowed us to address some important issues regarding the nature of CD4⁺ T cell responses elicited by the vaccine. We could unambiguously show that, whereas DR52b/ESO tetramer⁺ T cells were below detection limits in healthy donors as well as in patients prior to vaccination, they were clearly induced in remarkably similar proportions among different individuals following vaccination. Combination of staining with tetramers and antibodies directed against activation/differentiation markers allowed us to demonstrate that vaccine-induced CD4⁺ T cells were mostly composed of central and transitional memory cells, a phenotype that has been associated with protective memory responses to viruses (14, 15). In addition and importantly, we could demonstrate that most vaccine-induced CD4⁺ T cells were T helper polyfunctional cells of type I (18) and not suppressive Treg. Together, these results illustrate of the usefulness of molecularly defined class II/peptide tetramers to monitor tumor antigen-specific CD4 T cells, a crucial aspect in the development of anti-cancer vaccines. It is noteworthy that whereas DR52b/ESO tetramers allowed us to monitor vaccine-induced CD4⁺ T cells in 50% of vaccinated patients, the remaining 50% express another alternate DR molecule, DRB4*0101-0103, that has been also reported to present ESO-derived peptides (31), suggesting that the use of only two tetramers might be sufficient to monitor ESO-specific CD4⁺ T cells in the large majority of individuals.

In sum, the combination of a technical advance in the synthesis of class II/peptide tetramers (use of his-tagged peptides and affinity purification of peptide-loaded monomers prior to tetramerization) together with the use of frequently expressed alternate DR molecules, has the potential to significantly accelerate the development of reliable MHC class II/peptide tetramers, allowing the monitoring of CD4⁺ T cells specific for many other antigens in a variety of pathological conditions as well as in the course of immune interventions.

### REFERENCES

1. Altman, J. D., Moss, P. A. H., Goulder, P. J. R., Barouch, D. H., McHeyzer-Williams, M. G., Bell, J. I., McMichael, A. J., and Davis, M. M. 1996. Phenotypic analysis of antigen-specific T lymphocytes. Science. 274:94-96.
2. Guillaume, P., Dojcinovic, D., and Luescher, I. F. 2009. Soluble MHC-peptide complexes: tools for the monitoring of T cell responses in clinical trials and basic research. Cancer Immun 9:7.
3. Nepom, G. T., Buckner, J. H., Novak, E. J., Reichstetter, S., Reijonen, H., Gebe, J., Wang, R., Swanson, E., and Kwok, W. W. 2002. HLA class II tetramers: tools for direct analysis of antigen-specific CD4+ T cells. Arthritis Rheum 46:5-12.
4. Vollers, S. S., and Stern, L. J. 2008. Class II major histocompatibility complex tetramer staining: progress, problems, and prospects. Immunology 123:305-313.
5. Cecconi, V., Moro, M., Del Mare, S., Dellabona, P., and Casorati, G. 2008. Use of MHC class II tetramers to investigate CD4+ T cell responses: problems and solutions. Cytometry A 73:1010-1018.
6. Gnjatic, S., Nishikawa, H., Jungbluth, A. A., Gure, A. O., Ritter, G., Jager, E., Knuth, A., Chen, Y. T., and Old, L. J. 2006. NY-ESO-1: review of an immunogenic tumor antigen. Adv Cancer Res 95:1-30.
7. Cheever, M. A., Allison, J. P., Ferris, A. S., Finn, O. J., Hastings, B. M., Hecht, T. T., Mellman, I., Prindiville, S. A., Viner, J. L., Weiner, L. M., et al. 2009. The prioritization of cancer antigens: a national cancer institute pilot project for the acceleration of translational research. Clin Cancer Res 15:5323-5337.
8. Valmori, D., Souleimanian, N. E., Tosello, V., Bhardwaj, N., Adams, S., O'Neill, D., Pavlick, A., Escalon, J. B., Cruz, C. M., Angiulli, A., et al. 2007. Vaccination with NY-ESO-1 protein and CpG in Montanide induces integrated antibody/Th1 responses and CD8 T cells through cross-priming. Proc Natl Acad Sci USA 104:8947-8952.
9. Bioley, G., Dousset, C., Yeh, A., Dupont, B., Bhardwaj, N., Mears, G., Old, L. J., Ayyoub, M., and Valmori, D. 2009. Vaccination with recombinant NY-ESO-1 protein elicits immunodominant HLA-DR52b-restricted CD4+ T cell responses with a conserved T cell receptor repertoire. Clin Cancer Res 15:4467-4474.
10. Novak, E. J., Liu, A. W., Nepom, G. T., and Kwok, W. W. 1999. MHC class II tetramers identify peptide-specific human CD4(+) T cells proliferating in response to influenza A antigen. J Clin Invest 104:R63-67.
11. Dutoit, V., Rubio-Godoy, V., Doucey, M. A., Batard, P., Lienard, D., Rimoldi, D., Speiser, D., Guillaume, P., Cerottini, J. C., Romero, P., et al. 2002. Functional avidity of tumor antigen-specific CTL recognition directly correlates with the stability of MHC/peptide multimer binding to TCR. J Immunol 168:1167-1171.
12. Dutoit, V., Guillaume, P., Cerottini, J. C., Romero, P., and Valmori, D. 2002. Dissecting TCR-MHC/peptide complex interactions with A2/peptide multimers incorporating tumor antigen peptide variants: crucial role of interaction kinetics on functional outcomes. Eur J Immunol 32:3285-3293.
13. Texier, C., Pouvelle-Moratille, S., Busson, M., Charron, D., Menez, A., and Maillere, B. 2001. Complementarity and redundancy of the binding specificity of HLA-DRB1, -DRB3, -DRB4 and -DRB5 molecules. Eur J Immunol 31:1837-1846.
14. Okoye, A., Meier-Schellersheim, M., Brenchley, J. M., Hagen, S. I., Walker, J. M., Rohankhedkar, M., Lum, R., Edgar, J. B., Planer, S. L., Legasse, A., et al. 2007. Progressive CD4+ central memory T cell decline results in CD4+ effector memory insufficiency and overt disease in chronic SIV infection. J Exp Med 204:2171-2185.
15. Riou, C., Yassine-Diab, B., Van grevenynghe, J., Somogyi, R., Greller, L. D., Gagnon, D., Gimmig, S., Wilkinson, P., Shi, Y., Cameron, M. J., et al. 2007. Convergence of TCR and cytokine signaling leads to FOXO3a phosphorylation and drives the survival of CD4+ central memory T cells. J Exp Med 204:79-91.
16. Liu, W., Putnam, A. L., Xu-Yu, Z., Szot, G. L., Lee, M. R., Zhu, S., Gottlieb, P. A., Kapranov, P., Gingeras, T. R., Fazekas de St Groth, B., et al. 2006. CD127 expression inversely correlates with FoxP3 and suppressive function of human CD4+ T reg cells. J Exp Med 203:1701-1711.
17. Seddiki, N., Santner-Nanan, B., Martinson, J., Zaunders, J., Sasson, S., Landay, A., Solomon, M., Selby, W., Alexander, S. I., Nanan, R., et al. 2006. Expression of interleukin (IL)-2 and IL-7 receptors discriminates between human regulatory and activated T cells. J Exp Med 203:1693-1700.
18. Seder, R. A., Darrah, P. A., and Roederer, M. 2008. T-cell quality in memory and protection: implications for vaccine design. Nat Rev Immunol 8:247-258.
19. Garboczi, D. N., Hung, D. T., and Wiley, D. C. 1992. HLA-A2-peptide complexes: refolding and crystallization of molecules expressed in Escherichia coli and complexed with single antigenic peptides. Proc Natl Acad Sci USA 89:3429-3433.
20. Scott, C. A., Garcia, K. C., Carbone, F. R., Wilson, I. A., and Teyton, L. 1996. Role of chain pairing for the production of functional soluble IA major histocompatibility complex class II molecules. J Exp Med 183:2087-2095.
21. Kwok, W. W., Liu, A. W., Novak, E. J., Gebe, J. A., Ettinger, R. A., Nepom, G. T., Reymond, S. N., and Koelle, D. M. 2000. HLA-DQ tetramers identify epitope-specific T cells in peripheral blood of herpes simplex virus type 2-infected individuals: direct detection of immunodominant antigen-responsive cells. J Immunol 164:4244-4249.
22. Kozono, H., White, J., Clements, J., Marrack, P., and Kappler, J. 1994. Production of soluble MHC class II proteins with covalently bound single peptides. Nature 369:151-154.
23. Cotner, T., Charbonneau, H., Mellins, E., and Pious, D. 1989. mRNA abundance, rather than differences in subunit assembly, determine differential expression of HLA-DR beta 1 and -DR beta 3 molecules. J Biol Chem 264:11107-11111.
24. Sengar, D. P., Goldstein, R., Toye, B., and Hampton, N. 1994. Comprehensive typing of DR52 (DRB3)-associated DRB1 and DRB3 alleles by PCR-RFLP. Tissue Antigens 43:286-294.
25. Robinson, J., Waller, M. J., Parham, P., Bodmer, J. G., and Marsh, S. G. 2001. IMGT/HLA Database—a sequence database for the human major histocompatibility complex. Nucleic Acids Res 29:210-213.
26. Faner, R., James, E., Huston, L., Pujol-Borrel, R., Kwok, W. W., and Juan, M. 2010. Reassessing the role of HLA-DRB3 T-cell responses: evidence for significant expression and complementary antigen presentation. Eur J Immunol 40:91-102.
27. Cameron, T. O., Cochran, J. R., Yassine-Diab, B., Sekaly, R. P., and Stern, L. J. 2001. Cutting edge: detection of antigen-specific CD4+ T cells by HLA-DR1 oligomers is dependent on the T cell activation state. J Immunol 166:741-745.
28. Scriba, T. J., Purbhoo, M., Day, C. L., Robinson, N., Fidler, S., Fox, J., Weber, J. N., Klenerman, P., Sewell, A. K., and Phillips, R. E. 2005. Ultrasensitive detection and phenotyping of CD4+ T cells with optimized HLA class II tetramer staining. J Immunol 175:6334-6343.
29. Brown, J. H., Jardetzky, T. S., Gorga, J. C., Stern, L. J., Urban, R. G., Strominger, J. L., and Wiley, D. C. 1993. Three-dimensional structure of the human class II histocompatibility antigen HLA-DR1. Nature 364:33-39.
30. Dai, S., Crawford, F., Marrack, P., and Kappler, J. W. 2008. The structure of HLA-DR52c: comparison to other HLA-DRB3 alleles. Proc Natl Acad Sci USA 105:11893-11897.
31. Jager, E., Jager, D., Karbach, J., Chen, Y. T., Ritter, G., Nagata, Y., Gnjatic, S., Stockert, E., Arand, M., Old, L. J., et al. 2000. Identification of NY-ESO-1 epitopes presented by human histocompatibility antigen (HLA)-DRB4*0101-0103 and recognized by CD4(+) T lymphocytes of patients with NY-ESO-1-expressing melanoma. J Exp Med 191:625-630.
32. Fourneau, J. M., Cohen, H., and van Endert, P. M. 2004. A chaperone-assisted high yield system for the production of HLA-DR4 tetramers in insect cells. J Immunol Methods 285:253-264.
33. Ayyoub, M., Rimoldi, D., Guillaume, P., Romero, P., Cerottini, C., Valmori, D., and Speiser, D. 2003. Tumor-reactive SSX-2-specific CD8+ T cells are selectively expanded during immune responses to antigen expressing tumors in melanoma patients. Cancer Res 63:5601-5606.
34. Ayyoub, M., Deknuydt, F., Raimbaud, I., Dousset, C., Leveque, L., Bioley, G., and Valmori, D. 2009. Human memory FOXP3+ Tregs secrete IL-17 ex vivo and constitutively express the T(H)17 lineage-specific transcription factor RORgamma t. Proc Natl Acad Sci USA 106:8635-8640.

The entire contents of all of the references (including literature references, issued patents, published patent applications, and co-pending patent applications) cited throughout this application are hereby expressly incorporated by reference for the purposes or subject matter referenced herein.

### Example 3

Active elicitation of immune responses to tumor-specific antigens through vaccination is currently explored as a strategy that could complement standard cancer therapy to stabilize disease and prevent recurrence (1-3). One promising approach is to use molecularly defined synthetic vaccines incorporating well-characterized recombinant tumor antigens administered with strong adjuvants (4, 5). These vaccines can elicit integrated antibody and cellular immune responses, but their ability to eradicate cancer cells, particularly in the case of intracellular tumor antigens, relies on the elicitation of antigen specific T cells. Although cytotoxic CD8 T cells (CTL) are considered the main anti-tumor effector cells, CD4 T cell responses are key to the development of efficient anti-tumor immunity, both by providing help for the development of CTL and by directly exerting different effector functions (6-10). A rapid and hopefully successful development of anti-cancer vaccines is therefore dependent on the availability of methods that allow the efficient and reliable monitoring of vaccine induced tumor antigen-specific T cells. In this context, the development of soluble fluorescent MHC-peptide oligomers (commonly referred to as tetramers), allowing the direct visualization, enumeration and characterization of antigen specific T cells, has represented a major advance (11, 12). Hundreds of tetramers corresponding to different MHC class I alleles incorporating peptides from pathogen and self-antigens, including tumor antigens, have been generated and widely used in recent years (12-14). The development of MHC class II-peptide tetramers, instead, has been much more limited, and has been successful only in a minority of cases (12, 15-17).

NY-ESO-1 (ESO), a tumor-specific antigen of the cancer/testis group frequently expressed in tumors of different histological types but not in normal somatic tissues is an important candidate for the development of generic anti-cancer vaccines (18, 19). Several candidate anti-cancer vaccines using ESO-based immunogens are currently under trial (cancer Vaccine Collaborative: www.cancerresearch.org) (4, 20). Following vaccination with a recombinant ESO protein (rESO) administered with Montanide ISA-51 and CpG ODN 7909, we have obtained induction of CD4 T cell responses in 17/18 vaccinated patients (4). The majority of vaccine-induced CD4 T cells were directed against two immunodominant regions of ESO, corresponding to peptides 81-100 and 119-143. ESO₁₁₉₋₁₄₃ has been previously reported to bind to multiple MHC class II molecules (21, 22) and epitopes located in the ESO₁₁₉₋₁₄₃ region and recognized by specific CD4 T cells in the context of several HLA-DR molecules have been identified (21, 23, 24). We have generated tetramers of ESO₁₁₉₋₁₄₃ presented in the context of DR52b (DRB3*0202), an alternate DR molecule frequently expressed by Caucasians (25). Whereas the use of DR52b chains containing leucine zipper motifs was not sufficient, alone, for the successful generation of DR52b/ESO tetramers, we have shown that the use of ESO peptides bearing an amino-terminal His-tag, that allows the isolation of folded monomers by affinity purification, allows the generation of efficient tetramers (25).

In this study, we generated tetramers of DRB1*0101 (DR1) incorporating peptide ESO₁₁₉₋₁₄₃, using this strategy. We initially validated the DR1/ESO₁₁₉₋₁₄₃ tetramers on specific and control clones. We then assessed peptide-stimulated cultures from vaccinated patients expressing DR1, isolated DR1/ESO₁₁₉₋₁₄₃ tetramer+ cells by cell sorting and further characterized them functionally. Finally, we used the DR1/ESO₁₁₉₋₁₄₃ tetramers to assess vaccine-induced CD4 T cells ex vivo and characterize them phenotypically.

### Material and Methods

Generation of Fluorescent HLA-DR1/ESO Peptide Tetramers.

Soluble DR1 molecules were produced in D. mel-2 cells and purified by anti-HLA-DR (clone L243) immuno-affinity chromatography as previously described (25). The DR1 eluate was brought to the optimal peptide loading pH of 6.0 with 100 mM citric acid, loaded at a peptide to protein molar ratio of 50:1, at 28° C. for 24 hrs in the presence of a protease inhibitor cocktail (Roche) and 0.2% octyl β-D-glucopyranoside (Sigma) and then biotinylated using the BirA enzyme (Avidity). When DR1 molecules were loaded with untagged ESO peptides, complexes were directly purified by gel filtration in PBS pH 7.4, 100 mM NaCl on a Superdex S200 column (GE Healthcare Life Sciences) and the fractions corresponding to the monomeric pMHC complexes were pooled and concentrated. Alternatively, ESO peptides were extended at the N-terminus by a sequence containing 6 His residues and a linker (Ser-Gly-Ser-Gly, SEQ ID NO: 119). DR1/His tag-ESO peptide complexes were purified using a H isTrap HP 1 ml column (GE Healthcare Life Sciences) prior to purification by gel filtration. Finally, biotinylation and purity, as assessed by SDS-PAGE in an avidin shift assay, were >90%. Biotinylated DR1/peptide monomers were multimerized by mixing with small aliquots of streptavidin-PE (Invitrogen) up to the calculated 4:1 stoichiometry.

Patients Samples, Cells and Tissue Culture.

Peripheral blood samples were collected from cancer patients enrolled in a clinical trial of vaccination with rESO, Montanide ISA-51 and CpG 7909 (4) upon informed consent and approval by the Institutional Review Boards. MHC class II alleles were determined by high resolution molecular typing (24). L.DR1, DR1-transfected mouse cells kindly provided by Dr. Hassane M. Zarour (Department of Medicine and Melanoma Center, University of Pittsburgh Cancer Institute, Pittsburgh, Pa., USA), were maintained in complete RPMI medium containing gentamicin (Invitrogen) and periodically typed for HLA-DR1 expression. ESO₁₁₉₋₁₄₃-specific DR1-restricted CD4 T cell clones were obtained from post-vaccine samples from DR1+ patients as previously described (24). Clones were expanded by periodic (every 3-4 wk) stimulation with phytohemagglutinin (PHA, OXOID) and allogeneic irradiated PBMC and cultured in complete IMDM medium in the presence of rhIL-2 (100 IU/mL).

Assessment of ESO-Specific CD4 T Cells, Tetramer Staining and Flow Cytometric Analysis and Sorting.

For assessment of specific CD4 T cell responses following in vitro stimulation, CD4+ cells were enriched from PBMC by magnetic cell sorting (Miltenyi Biotec Inc.), stimulated with irradiated autologous APC in the presence of ESO peptides, as indicated, rhIL-2 and rhIL-7 as previously described (24) and maintained in culture during 10-15 days prior to tetramer staining. Peptide stimulated cultures and specific monoclonal and polyclonal populations were incubated with tetramers at a final concentration of 3 μg/ml for 1 hr at 37° C., unless otherwise indicated, in complete IMDM medium, washed and then stained with CD4 (BD Biosciences) or TCR V13 (Beckman Coulter) specific mAb in PBS, 5% FCS for 15 minutes at 4° C. and analyzed by flow cytometry (FACSAria, BD Biosciences). In order to generate specific polyclonal T cell populations, tetramer+ cells within peptide-stimulated cultures were sorted by flow cytometry (FACSAria, BD Biosciences) and expanded by stimulation with PHA and irradiated allogeneic PBMC in the presence of rhIL-2 (26). For ex vivo enumeration and phenotyping of specific cells, CD4+ cells enriched from PBMC were rested overnight, incubated with tetramers (3 μg/ml) for 2 hrs at 37° C. and then stained with CD4−, CD45RA− (BD Biosciences) and CCR7− (Miltenyi Biotec Inc.) specific mAb and analyzed by flow cytometry.

Antigen Recognition Assays.

DR1+ ESO-specific monoclonal or polyclonal CD4 T cell populations were stimulated in the absence or presence of ESO peptides (2 μM) or PMA (100 ng/ml) and ionomycin (1 μg/ml), as indicated, and cytokine production was assessed in a standard 4 hr intracellular cytokine staining assay using mAb specific for IFN-γ, TNF- [symbol] , IL-2, IL-4, IL-10 (BD Biosciences) and IL-17 (eBiosciences) and flow cytometric analysis, as previously described (24, 27). In order to assess DR1 restriction of monoclonal and polyclonal ESO-specific populations, they were incubated for 4 hrs with L.DR1 cells or with untransfected mouse fibroblasts that have been pulsed with peptide ESO₁₁₉₋₁₄₃ for 1 hr at 37° C. and washed 3 times, and IFN-γ production was assessed by intracellular staining and flow cytometric analysis. In other experiments, specific polyclonal cultures were incubated for 24 hrs with either L.DR1 cells and serial dilutions of ESO peptides or monocyte derived dendritic cells pre-incubated overnight with serial dilutions of rESO, and IFN-γ was measured by ELISA in 24 hr culture supernatants, as previously described (4, 24).

### Results and Discussion

Generation and Validation of DRB1*0101/ESO₁₁₉₋₁₄₃ Tetramers.

Direct assessment with fluorescent MHC class II tetramers incorporating immunodominant peptides from frequently expressed tumor antigens is an attractive approach for the monitoring of anti-tumor CD4 T cells. At variance with MHC class I/peptide tetramers, originally developed in 1996 (11) that have since been generated for a large number of alleles incorporating a variety of peptides, including ones from tumor antigens, the development of MHC class II/peptide tetramers has proven significantly more difficult (12, 15-17). Among limiting factors are the high polymorphism of MHC class II molecules, the often low binding affinity of peptides from tumor/self antigens, and the structural characteristics of MHC class II molecules. Namely, because MHC class II αβ chain monomers are unstable in solution, one strategy to improve tetramer generation has consisted in adding leucine zippers to facilitate α,β pairing (28). MHC class II αβ chains incorporating leucine zippers, however, can form stable complexes also in the absence of bound peptides, which can lead to the generation of tetramers formed by “empty” MHC class II molecules. While attempting to generate tetramers of the alternate DR molecule DR52b incorporating peptide ESO₁₁₁₉₋₁₄₃, we found that the use of leucine zipper containing DR52b molecules alone was insufficient for the generation of tetramers able to significantly stain specific CD4 T cells. We therefore implemented the approach by using His-tagged peptides, allowing the isolation of folded MHC class II/peptide monomers by affinity purification, which resulted in the generation of efficient DR52b/ESO tetramers (25). In this study, we used the same strategy to generate tetramers of DRB1*0101 (DR1) incorporating ESO₁₁₉₋₁₄₃. To validate the DR1/ESO₁₁₉₋₁₄₃ tetramers, we initially assessed them on a specific clone (FIG. 17A) obtained from a DR1+ patient who had been immunized with the rESO vaccine (4). As shown in FIG. 17B, the tetramers efficiently stained the specific clone but not an irrelevant clone used as control. To optimize the tetramer staining conditions, we assessed the effect of tetramer concentration, incubation time and temperature on specific and control clones. We obtained significant staining of specific clones with relatively low doses of tetramer (1 μg/ml). The staining intensity increased with the dose of tetramer, up to 30 μg/ml, without reaching a plateau (FIG. 17B). Staining of specific clones was more efficient at high temperature (37° C.) and after prolonged incubation times (FIG. 17C). Thus, the use of leucine zipper-containing DR1 molecules and His-tagged ESO peptides resulted in the generation of efficient tetramers. Because the loading efficiency of MHC class II/peptide complexes, and therefore the need for using His-tagged peptides, could significantly vary for different MHC class II molecules and peptides, we also prepared DR1/ESO tetramers using untagged peptides. As shown in FIG. 17D, DR1/ESO tetramers generated with the untagged peptide ESO₁₁₉₋₁₄₃ also stained ESO specific clones, although with slightly lower efficiency as compared to DR1/ESO tetramers prepared using His-tagged peptides. Thus, in contrast to DR52b/ESO tetramers, the use of His-tagged peptides was helpful but not indispensable for the generation of DR1/ESO tetramers.

Assessment of Peptide-Stimulated Cultures from Vaccinated Patients Using DRB1*0101/ESO₁₁₉₋₁₄₃ Tetramers.

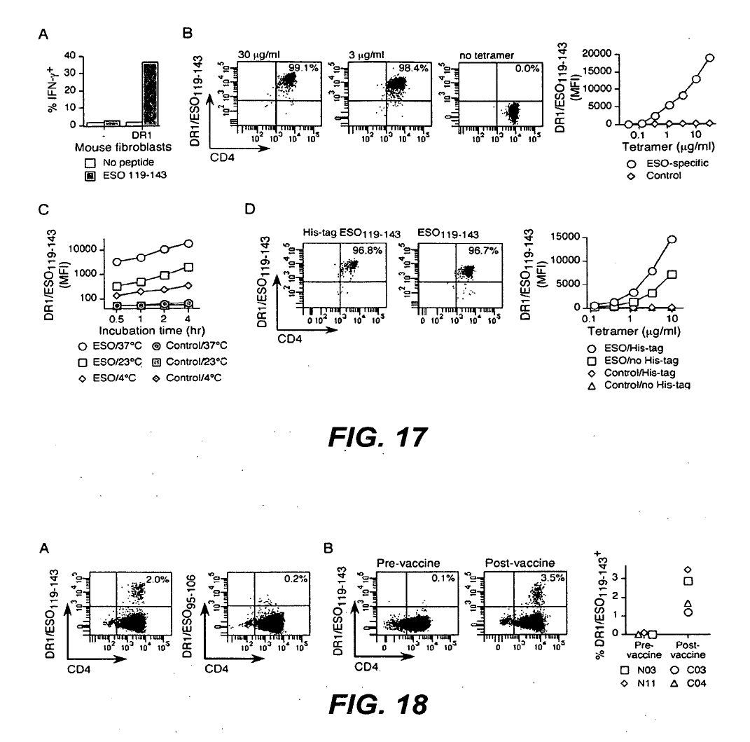

To evaluate vaccine-induced CD4 T cells in DR1+ immunized patients, we initially stained post-vaccine CD4 T cells from patient N03 (a high responder to the vaccine, expressing DRB1*0101) previously stimulated in vitro for 12 days with a pool of long overlapping peptides spanning the entire ESO sequence (4), with DR1/ESO₁₁₉₋₁₄₃ tetramers during 1 hr at 37° C. As illustrated in FIG. 18A, this analysis identified a significant proportion of DR1/ESO₁₁₉₋₁₄₃ tetramers+ CD4 T cells in the culture. DR1 tetramers incorporating peptide ESO₉₅₋₁₀₆, used as an internal control, failed to identify significant proportions of tetramer+ cells. In a separate experiment, we stimulated post vaccine samples from patient N03 and from 3 additional vaccinated patients expressing DR1 alleles (N11 and C04 also expressing DRB1*0101 and C03 expressing DRB1*0103) with peptide ESO₁₁₉₋₁₄₃ alone and assessed them 12 days later with the DR1/ESO₁₁₉₋₁₄₃ tetramers. As illustrated in FIG. 18B, we detected significant proportions of tetramer+ cells in cultures from all patients. DR1/ESO₁₁₉₋₁₄₃ tetramer+ cells had clearly been induced by vaccination, as they were not detectable at significant levels in pre-vaccine samples stimulated in the same conditions.

Isolation and Characterization of Vaccine-Induced DR1/ESO₁₁₉₋₁₄₃ Tetramer+ Cells.

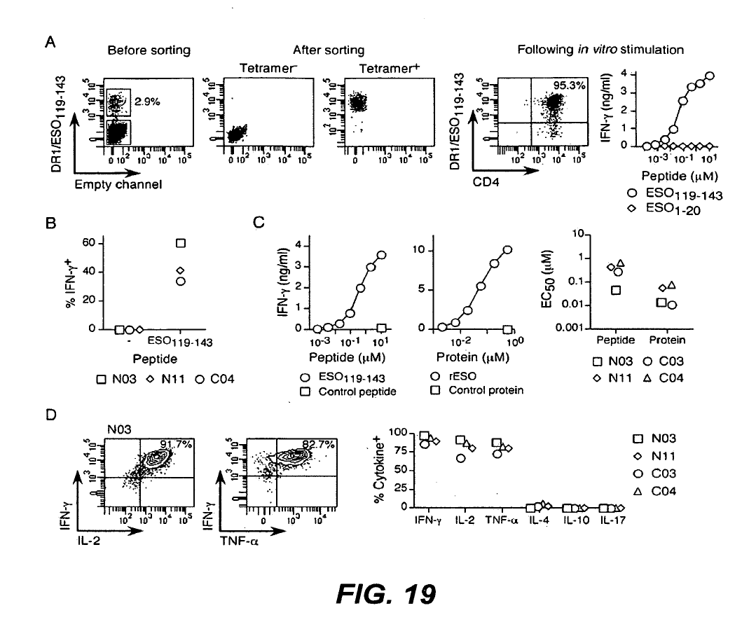

To assess vaccine-induced DR1/ESO₁₁₉₋₁₄₃ tetramer+ cells, we isolated them by flow cytometry cell sorting and expanded them in vitro, as polyclonal monospecific cultures (FIG. 19A). Isolated tetramer+ cells specifically recognized peptide ESO₁₁₉₋₁₄₃ but not a control ESO peptide (FIG. 19A). Antigen recognition by polyclonal monospecific tetramer+ cells was restricted by DR1, as efficient antigen presentation was obtained using DR1-tranfected mouse cells preincubated with peptide ESO₁₁₉₋₁₄₃ (FIG. 19B). To further characterize vaccine-induced DR1/ESO₁₁₉₋₁₄₃ tetramer+ cells, we assessed their capacity to efficiently recognize the full length recombinant ESO protein (rESO) processed and presented by autologous APC. To this purpose we generated monocyte-derived dendritic cells (moDC) by culturing autologous CD14+ cells with GM-CSF and IL-4 as described (4), incubated them with serial dilutions of rESO and tetramer+ cells and assessed IFN-γ secretion in the culture supernatant. As shown in FIG. 19C, tetramer+ cells recognized rESO processed and presented by autologous moDC with high efficiency as half-maximal recognition was obtained at a concentration of rESO similar to that of ESO₁₁₉₋₁₄₃ peptide presented by DR1-expressing APC. To assess the type of vaccine-induced DR1/ESO₁₁₉₋₁₄₃ tetramer+ cells with respect to cytokine secretion, we stimulated them with PMA and ionomycin, permeabilized and stained them with mAb specific for signature cytokines produced by different TH cell subsets. As illustrated in FIG. 19D, tetramer+ cells displayed a typical TH1 profile as they secreted IFN-γ, IL-2 and TNF-α, but not IL-4, IL-10 or IL-17.

DR1/ESO₁₁₉₋₁₄₃ Tetramer+ Cells Use a Conserved TCR Repertoire.

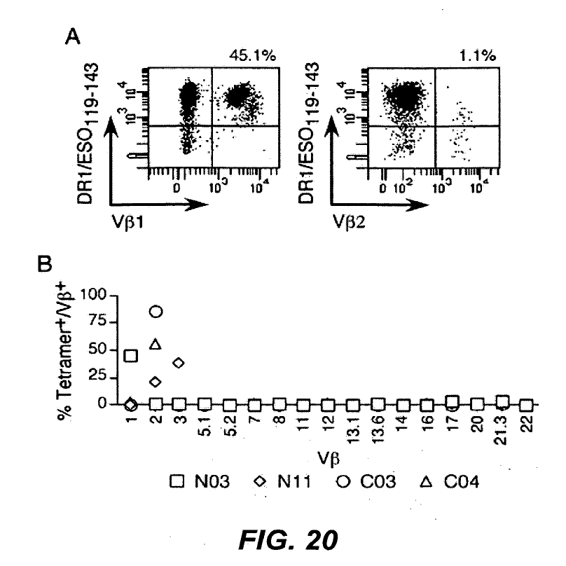

T cells recognizing defined MHC/peptide complexes often exhibit conserved features including the use of defined variable regions of the TCR α and β chains (Vα and Vβ). To address if DR1/ESO₁₁₉₋₁₄₃ tetramer+ cells exhibited such conserved features, we assessed the polyclonal monospecific tetramer+ populations from vaccinated patients with a panel of anti-V13 mAb covering about 50% of the human TCR repertoire. Examples of co-staining with anti-Vβ mAb and tetramers are shown in FIG. 20A and a summary of the data obtained is reported in FIG. 20B. We found a frequent usage of several Vβ segments, including Vβ1, Vβ2 and Vβ3. Vβ1 tetramer+ cells were prevalent in the culture of patient N03, representing half of the total population. The large majority of tetramer+ cells in the culture of patient C03 and a significant proportion of tetramer+ cells in the cultures of two other patients, N11 and C04, used Vβ2. Finally, about half of tetramer+ cells in the culture of patient N11 used Vβ3. Thus, DR1/ESO₁₁₉₋₁₄₃ tetramer+ cells frequently used few selected Vβ regions, indicating the presence of a conserved TCR repertoire.

Assessment of the Minimal ESO Peptide Optimally Recognized by DR1/ESO₁₁₉₋₁₄₃ Tetramer+ Cells.

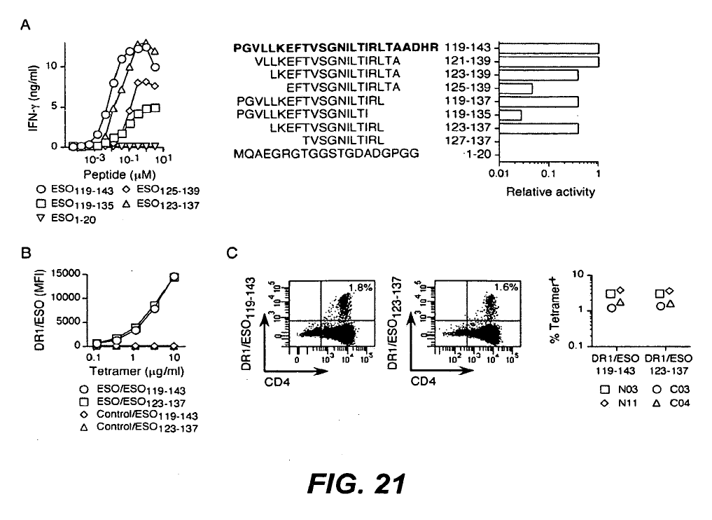

In a previous study assessing ESO₁₁₉₋₁₄₃ binding to several MHC class II alleles, including DR1, the 15-mer ESO₁₂₃₋₁₃₇ showed a binding affinity for DR1 similar to that of ESO₁₁₉₋₁₄₃ (21). To better define the DR1 epitope with respect to recognition by specific T cells, we assessed the recognition of truncated peptides within the ESO₁₁₉₋₁₄₃ region by tetramer+ T cells. NH2-terminal truncations up to amino acid 123 did not significantly affect recognition by tetramer+ T cells (FIG. 21A). Further truncation, however, significantly reduced recognition. Similarly, COOH-terminal truncations up to amino acid 137 did not significantly affect recognition, whereas further truncation reduced it. This analysis identified ESO₁₂₃₋₁₃₇ as the minimal peptide optimally recognized by DR1/ESO₁₁₉₋₁₄₃ tetramer+ CD4 T cells. In line with these results, DR1 tetramers incorporating peptide ESO₁₂₃₋₁₃₇ stained specific clones with the same efficiency as compared to DR1/ESO₁₁₉₋₁₄₃ tetramers (FIG. 21B) and identified similar proportions of CD4 tetramer+ cells in peptide-stimulated cultures from post-vaccine samples (FIG. 21C).

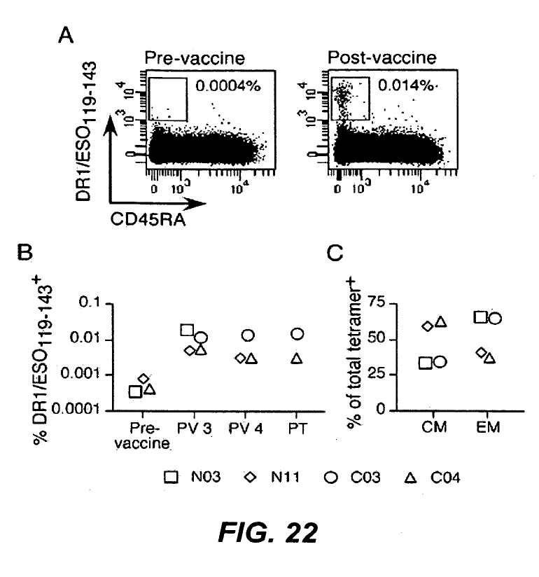

Ex vivo assessment of the frequency and phenotype of vaccine-induced ESO-specific CD4 T cell responses with DR1/ESO₁₁₉₋₁₄₃ tetramers. The relatively high frequency of DR1/ESO₁₁₉₋₁₄₃ tetramer+ CD4 T cells detected in peptide-stimulated cultures from vaccinated patients encouraged us to attempt assessing the frequency and phenotype of vaccine-induced CD4 T cells in DR1 expressing patients ex vivo. To this end, for each patient, we isolated CD4 T cells by magnetic cell sorting from samples taken prior to and at different time points after vaccination, when available, and stained them with DR1/ESO tetramers together with antibodies directed against markers that distinguish CD4 T cells according to their differentiation stage (29). For 3 of the 4 patients, samples taken prior to vaccination were available. The frequency of DR1/ESO tetramer+ cells among memory (CD45RA−) CD4 T cells in pre-vaccine samples was below detection limits (<1:100 000) (FIGS. 22A and 22B). In contrast, in post-vaccine samples from all patients taken after 3 vaccine injections (PV 3) DR1/ESO tetramer+ cells were detectable at a frequency that was variable among different patients and was in average of about 1:10 000 memory CD4 T cells. For 3 patients for whom additional samples taken after 4 vaccine injections (PV 4) were available, DR1/ESO tetramer+ cells were detectable at a frequency that was, for each patient, comparable to that detected after 3 injections. For 2 patients, C03 and C04, additional samples taken 4 and 5 months respectively after the 4th and last injection (post-treatment, PT) were also available. In these samples, DR1/ESO tetramer+ cells were still detectable at a frequency similar, for each patient, to that detected one week after the last injection (PV 4). Vaccine-induced DR1/ESO tetramer+ cells included both central memory (CCR7+) representing “reservoir” memory populations (30, 31) and effector memory populations (CCR7−) (FIG. 22C).

In conclusion, assessment of vaccine-induced CD4 T cells using DR1/ESO tetramers confirmed the ability of the ESO vaccine to induce strong and long lasting CD4 T cell memory responses of TH 1 type, that are generally associated with efficient anti-tumor responses. The high efficiency and specificity of the staining obtained with the DR1/ESO tetramers allowed the direct ex vivo detection of specific cells among total CD4 T cells, without the need for enrichment steps used in previous studies (28, 32). It is noteworthy that the frequency of vaccine-induced ESO-specific CD4 T cells detected ex vivo (in average 1:10 000 memory cells) is in the same range of ex vivo frequencies of previously reported DR1-restricted CD4 T cells specific for viral epitopes (28, 32). The generation and validation of DR1/ESO tetramers reported in this study encourage their further use for the evaluation of CD4 T cells specific for this important tumor antigen in the context of spontaneous or vaccine-induced immune responses in DR1 expressing patients.

### REFERENCES

1. Simpson A J, Caballero O L, Jungbluth A, Chen Y T, and Old L J. Cancer/testis antigens, gametogenesis and cancer. Nat Rev Cancer 2005; 5:615-25.
2. Finn O J. Cancer immunology. N Engl J Med 2008; 358:2704-15.
3. Caballero O L and Chen Y T. Cancer/testis (CT) antigens: potential targets for immunotherapy. Cancer Sci 2009; 100:2014-21.
4. Valmori D, Souleimanian N E, Tosello V, et al. Vaccination with NY-ESO-1 protein and CpG in Montanide induces integrated antibody/Th1 responses and CD8 T cells through cross-priming. Proc Natl Acad Sci USA 2007; 104:8947-52.
5. Vollmer J and Krieg A M. Immunotherapeutic applications of CpG oligodeoxynucleotide TLR9 agonists. Adv Drug Deliv Rev 2009; 61:195-204.
6. Pardoll D M and Topalian S L. The role of CD4+ T cell responses in antitumor immunity. Curr Opin Immunol 1998; 10:588-94.
7. Mumberg D, Monach P A, Wanderling S, et al. CD4(+) T cells eliminate MHC class II-negative cancer cells in vivo by indirect effects of IFN-gamma. Proc Natl Acad Sci USA 1999; 96:8633-8.
8. Toes R E, Ossendorp F, Offring a R, and Melief C J. CD4 T cells and their role in antitumor immune responses. J Exp Med 1999; 189:753-6.
9. Hung K, Hayashi R, Lafond-Walker A, Lowenstein C, Pardoll D, and Levitsky H. The central role of CD4(+) T cells in the antitumor immune response. J Exp Med 1998; 188:2357-68.
10. Hunder N N, Wallen H, Cao J, et al. Treatment of metastatic melanoma with autologous CD4+ T cells against NY-ESO-1. N Engl J Med 2008; 358:2698-703.
11. Altman J D, Moss P A H, Goulder P J R, et al. Phenotypic analysis of antigen-specific T lymphocytes. Science. 1996; 274:94-6.
12. Guillaume P, Dojcinovic D, and Luescher I F. Soluble MHC-peptide complexes: tools for the monitoring of T cell responses in clinical trials and basic research. Cancer Immun 2009; 9:7.
13. Bioley G, Guillaume P, Luescher I, et al. HLA class I-associated immunodominance affects CTL responsiveness to an ESO recombinant protein tumor antigen vaccine. Clin Cancer Res 2009; 15:299-306.
14. Bioley G, Guillaume P, Luescher I, et al. Vaccination with a recombinant protein encoding the tumor-specific antigen NY-ESO-1 elicits an A2/157-165-specific CTL repertoire structurally distinct and of reduced tumor reactivity than that elicited by spontaneous immune responses to NY-ESO-1-expressing Tumors. J Immunother 2009; 32:161-8.
15. Nepom G T, Buckner J H, Novak E J, et al. HLA class II tetramers: tools for direct analysis of antigen-specific CD4+ T cells. Arthritis Rheum 2002; 46:5-12.
16. Vollers S S and Stern L J. Class II major histocompatibility complex tetramer staining: progress, problems, and prospects. Immunology 2008; 123:305-13.
17. Cecconi V, Moro M, Del Mare S, Dellabona P, and Casorati G. Use of MHC class II tetramers to investigate CD4+ T cell responses: problems and solutions. Cytometry A 2008; 73:1010-8.
18. Chen Y T, Scanlan M J, Sahin U, et al. A testicular antigen aberrantly expressed in human cancers detected by autologous antibody screening. Proc Natl Acad Sci USA 1997; 94:1914-8.
19. Cheever M A, Allison J P, Ferris A S, et al. The prioritization of cancer antigens: a national cancer institute pilot project for the acceleration of translational research. Clin Cancer Res 2009; 15:5323-37.
20. Jager E, Karbach J, Gnjatic S, et al. Recombinant vaccinia/fowlpox NY-ESO-1 vaccines induce both humoral and cellular NY-ESO-1-specific immune responses in cancer patients. Proc Natl Acad Sci USA 2006; 103:14453-8.
21. Zarour H M, Maillere B, Brusic V, et al. NY-ESO-1 119-143 is a promiscuous major histocompatibility complex class II T-helper epitope recognized by Th1- and Th2-type tumor-reactive CD4+ T cells. Cancer Res 2002; 62:213-8.
22. Valmori D, Souleimanian N E, Hesdorffer C S, Old L J, and Ayyoub M. Quantitative and qualitative assessment of circulating NY-ESO-1 specific CD4+ T cells in cancer free individuals. Clin Immunol 2005; 117:161-7.
23. Ayyoub M, Souleimanian N E, Godefroy E, et al. A phenotype based approach for the immune monitoring of NY-ESO-1 specific CD4+ T cell responses in cancer patients. Clin Immunol 2006; 118:188-94.
24. Bioley G, Dousset C, Yeh A, et al. Vaccination with recombinant NY-ESO-1 protein elicits immunodominant HLA-DR52b-restricted CD4+ T cell responses with a conserved T cell receptor repertoire. Clin Cancer Res 2009; 15:4467-74.
25. Ayyoub M, Dojcinovic D, Pignon P, et al. Monitoring of NY-ESO-1 specific CD4+ T cells using molecularly defined MHC class II/His-tag-peptide tetramers. Proc Natl Acad Sci USA 2010; 107:7437-42.
26. Ayyoub M, Rimoldi D, Guillaume P, et al. Tumor-reactive SSX-2-specific CD8+ T cells are selectively expanded during immune responses to antigen expressing tumors in melanoma patients. Cancer Res 2003; 63:5601-6.
27. Ayyoub M, Deknuydt F, Raimbaud I, et al. Human memory FOXP3+ Tregs secrete IL-17 ex vivo and constitutively express the T(H)17 lineage-specific transcription factor RORgamma t. Proc Natl Acad Sci USA 2009; 106:8635-40.
28. Novak E J, Liu A W, Nepom G T, and Kwok W W. MHC class II tetramers identify peptide-specific human CD4(+) T cells proliferating in response to influenza A antigen. J Clin Invest 1999; 104:R63-7.
29. Sallusto F, Lenig D, Forster R, Lipp M, and Lanzavecchia A. Two subsets of memory T lymphocytes with distinct homing potentials and effector functions. Nature 1999; 401:708-12.
30. Riou C, Yassine-Diab B, Van grevenynghe J, et al. Convergence of TCR and cytokine signaling leads to FOXO3a phosphorylation and drives the survival of CD4+ central memory T cells. J Exp Med 2007; 204:79-91.
31. Okoye A, Meier-Schellersheim M, Brenchley J M, et al. Progressive CD4+ central memory T cell decline results in CD4+ effector memory insufficiency and overt disease in chronic SIV infection. J Exp Med 2007; 204:2171-85.
32. Scriba T J, Purbhoo M, Day C L, et al. Ultrasensitive detection and phenotyping of CD4+ T cells with optimized HLA class II tetramer staining. J Immunol 2005; 175:6334-43.

The entire contents of all of the references (including literature references, issued patents, published patent applications, and co-pending patent applications) cited throughout this application are hereby expressly incorporated by reference for the purposes or subject matter referenced herein.

We claim:

1. An isolated immunostimulatory NY-ESO-1 peptide that can specifically bind an MHC class II molecule, and that, when bound to a MHC class II molecule, can specifically bind to a DRB3*0202 (DR52b) or DRB1*0101 (DR1) restricted CD4⁺ T cell, the NY-ESO-1 peptide comprising at least 9 contiguous amino acids of NY-ESO-1 (SEQ ID NO:1).

2. The isolated immunostimulatory NY-ESO-1 peptide of claim 1, the peptide comprising an amino acid sequence selected from the group consisting of peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1.

3.-6. (canceled)

7. An isolated peptide polytope, comprising
the NY-ESO-1 peptide of claim 1, and
at least one additional DR52b or DR1 restricted tumor antigen epitope.

8.-30. (canceled)

31. An isolated peptide-loaded MHC class II molecule, comprising
an MHC class II alpha chain,
an MHC class II beta chain, and
a tagged MHC-class II binding peptide.

32. (canceled)

33. The isolated peptide-loaded MHC class II molecule of claim 31, wherein the MHC class II protein comprises a DR52b or DR1 beta chain and/or is encoded by a DRB3*0202 or DRB1*0101 allele.

34. The isolated peptide-loaded MHC class II molecule of claim 31, wherein the tagged peptide
is a tagged NY-ESO-1 peptide comprising an amino acid sequence chosen from the sequences provided in SEQ ID NOs 2-60, and/or
comprising 9-25 contiguous amino acids of NY-ESO-1 (SEQ ID NO: 1), and/or
an amino acid sequence selected from the group consisting of peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1.

35. (canceled)

36. The isolated peptide-loaded MHC class II molecule of claim 31, wherein the MHC class II molecule is linked to a ligand of a multivalent binding molecule.

37. (canceled)

38. (canceled)

39. The isolated peptide-loaded MHC class II molecule of claim 36, wherein the ligand binds to a multivalent binding molecule, and, optionally, wherein the multivalent binding molecule binds at least one additional MHC class II molecule, wherein each additional MHC class II molecule is optionally peptide-loaded.

40.-48. (canceled)

49. An isolated MHC class II multimer, comprising
a multivalent binding molecule,
an isolated NY-ESO-1 peptide-loaded MHC class II molecule, comprising
an MHC class II molecule, comprising a beta chain encoded by a DRB3 or a DRB1 allele, and bound to a NY-ESO-1 peptide, the NY-ESO-1 peptide comprising
9-25 contiguous amino acids of NY-ESO-1 (SEQ ID NO: 1), and/or
an amino acid sequence selected from the group consisting of peptides of at least 9 amino acid residues starting at residue 119, 120, 121, 122, 123, 124, 125, 126, or 127 and/or ending at residue 134, 135, 136, 137, 138, 139, 140, 141, 142, or 143 of SEQ ID NO: 1,
wherein the MHC class II molecule is linked to a ligand of the multivalent binding molecule, and
at least one additional MHC class II molecule linked to a ligand of the multivalent binding molecule, wherein each of the at least one additional MHC class II molecule is optionally peptide-loaded, and
wherein the ligands bind to the multivalent binding molecule.

50. The isolated MHC class II multimer of claim 49,
wherein the multimer is a tetramer, comprising three additional MHC class II molecules linked to a ligand of the multivalent binding molecule.

51. The isolated MHC class II multimer of claim 49, wherein the ligand is biotin and the multivalent binding molecule is streptavidin or avidin.

52. The isolated MHC class II multimer of claim 49, wherein a MHC class II molecule of the multimer or the tetramer is loaded with a NY-ESO-1 peptide comprising an amino acid sequence starting at residue 123 and ending at residue 137 of SEQ ID NO: 1.

53. (canceled)

54. (canceled)

55. The isolated MHC class II multimer of claim 49, wherein the multimer or tetramer is labeled with a detectable label.

56.-62. (canceled)

63. An isolated MHC class II multimer, comprising
a multivalent binding molecule,
an isolated peptide-loaded MHC class II molecule, comprising
an MHC class II alpha chain,
an MHC class II beta chain, and
a tagged MHC-class II binding peptide,
wherein the MHC class II molecule is linked to a ligand of the multivalent binding molecule, and
at least one additional MHC class II molecule linked to a ligand of the multivalent binding molecule, wherein each of the at least one additional MHC class II molecule is optionally peptide-loaded, and
wherein the ligands of the isolated peptide-loaded MHC class II molecule and of the at least one additional MHC class II molecule bind to the multivalent binding molecule.

64. The isolated MHC class II multimer of claim 63, wherein the MHC class II multimer is a MHC class II tetramer, comprising
three additional MHC class II molecule linked to a ligand of the multivalent binding molecule.

65. The isolated MHC class II multimer or tetramer of claim 63 of claim 63, wherein the ligand is biotin and the multivalent binding molecule is streptavidin or avidin.

66. (canceled)

67. The isolated MHC class II multimer or tetramer of claim 63, wherein the multimer or tetramer is labeled with a detectable label.

68.-132. (canceled)

133. A method of measuring an immune response to a vaccination, comprising
obtaining a biological sample comprising a T-cell from a subject, wherein the subject has been administered a composition comprising an epitope of an antigen,
contacting the T-cell with an MHC class II multimer, the MHC class II multimer comprising
a plurality of MHC class II molecules, wherein at least one of the plurality of MHC class II molecules is loaded with a tagged peptide comprising an epitope of the antigen, and
detecting binding of the MHC class II multimer to the T-cell.

134.-205. (canceled)

206. A kit, comprising
an isolated MHC class II molecule, multimer, or tetramer, and/or
the isolated immunostimulatory NY-ESO-1 peptide of claim 1.

207.-210. (canceled)

211. An isolated DR MHC class II molecule, comprising
a DR beta chain, wherein the beta chain comprises an extracellular part of a DR52b protein fused to a leucine zipper sequence, and
a DR alpha chain, wherein the alpha chain comprises an extracellular part of a DR MHC class II alpha chain fused to a leucine zipper sequence that binds to the leucine zipper sequence fused to the beta chain.

212.-219. (canceled)


SEQUENCE LISTING (145 sequences)

SEQ ID NO: 1 - protein, 180 aa, Artificial Sequence
  synthetic peptide
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 2 - protein, 25 aa, Artificial Sequence
  synthetic peptide
PGVLLKEFTVSGNILTIRLTAADHR

SEQ ID NO: 3 - protein, 24 aa, Artificial Sequence
  synthetic peptide
PGVLLKEFTVSGNILTIRLTAADH

SEQ ID NO: 4 - protein, 23 aa, Artificial Sequence
  synthetic peptide
PGVLLKEFTVSGNILTIRLTAAD

SEQ ID NO: 5 - protein, 22 aa, Artificial Sequence
  synthetic peptide
PGVLLKEFTVSGNILTIRLTAA

SEQ ID NO: 6 - protein, 21 aa, Artificial Sequence
  synthetic peptide
PGVLLKEFTVSGNILTIRLTA

SEQ ID NO: 7 - protein, 20 aa, Artificial Sequence
  synthetic peptide
PGVLLKEFTVSGNILTIRLT

SEQ ID NO: 8 - protein, 19 aa, Artificial Sequence
  synthetic peptide
PGVLLKEFTVSGNILTIRL

SEQ ID NO: 9 - protein, 18 aa, Artificial Sequence
  synthetic peptide
PGVLLKEFTVSGNILTIR

SEQ ID NO: 10 - protein, 24 aa, Artificial Sequence
  synthetic peptide
GVLLKEFTVSGNILTIRLTAADHR

SEQ ID NO: 11 - protein, 23 aa, Artificial Sequence
  synthetic peptide
GVLLKEFTVSGNILTIRLTAADH

SEQ ID NO: 12 - protein, 22 aa, Artificial Sequence
  synthetic peptide
GVLLKEFTVSGNILTIRLTAAD

SEQ ID NO: 13 - protein, 21 aa, Artificial Sequence
  synthetic peptide
GVLLKEFTVSGNILTIRLTAA

SEQ ID NO: 14 - protein, 20 aa, Artificial Sequence
  synthetic peptide
GVLLKEFTVSGNILTIRLTA

SEQ ID NO: 15 - protein, 19 aa, Artificial Sequence
  synthetic peptide
GVLLKEFTVSGNILTIRLT

SEQ ID NO: 16 - protein, 18 aa, Artificial Sequence
  synthetic peptide
GVLLKEFTVSGNILTIRL

SEQ ID NO: 17 - protein, 17 aa, Artificial Sequence
  synthetic peptide
GVLLKEFTVSGNILTIR

SEQ ID NO: 18 - protein, 23 aa, Artificial Sequence
  synthetic peptide
VLLKEFTVSGNILTIRLTAADHR

SEQ ID NO: 19 - protein, 22 aa, Artificial Sequence
  synthetic peptide
VLLKEFTVSGNILTIRLTAADH

SEQ ID NO: 20 - protein, 21 aa, Artificial Sequence
  synthetic peptide
VLLKEFTVSGNILTIRLTAAD

SEQ ID NO: 21 - protein, 20 aa, Artificial Sequence
  synthetic peptide
VLLKEFTVSGNILTIRLTAA

SEQ ID NO: 22 - protein, 19 aa, Artificial Sequence
  synthetic peptide
VLLKEFTVSGNILTIRLTA

SEQ ID NO: 23 - protein, 18 aa, Artificial Sequence
  synthetic peptide
VLLKEFTVSGNILTIRLT

SEQ ID NO: 24 - protein, 17 aa, Artificial Sequence
  synthetic peptide
VLLKEFTVSGNILTIRL

SEQ ID NO: 25 - protein, 16 aa, Artificial Sequence
  synthetic peptide
VLLKEFTVSGNILTIR

SEQ ID NO: 26 - protein, 22 aa, Artificial Sequence
  synthetic peptide
LLKEFTVSGNILTIRLTAADHR

SEQ ID NO: 27 - protein, 21 aa, Artificial Sequence
  synthetic peptide
LLKEFTVSGNILTIRLTAADH

SEQ ID NO: 28 - protein, 20 aa, Artificial Sequence
  synthetic peptide
LLKEFTVSGNILTIRLTAAD

SEQ ID NO: 29 - protein, 19 aa, Artificial Sequence
  synthetic peptide
LLKEFTVSGNILTIRLTAA

SEQ ID NO: 30 - protein, 18 aa, Artificial Sequence
  synthetic peptide
LLKEFTVSGNILTIRLTA

SEQ ID NO: 31 - protein, 17 aa, Artificial Sequence
  synthetic peptide
LLKEFTVSGNILTIRLT

SEQ ID NO: 32 - protein, 16 aa, Artificial Sequence
  synthetic peptide
LLKEFTVSGNILTIRL

SEQ ID NO: 33 - protein, 15 aa, Artificial Sequence
  synthetic peptide
LLKEFTVSGNILTIR

SEQ ID NO: 34 - protein, 21 aa, Artificial Sequence
  synthetic peptide
LKEFTVSGNILTIRLTAADHR

SEQ ID NO: 35 - protein, 20 aa, Artificial Sequence
  synthetic peptide
LKEFTVSGNILTIRLTAADH

SEQ ID NO: 36 - protein, 19 aa, Artificial Sequence
  synthetic peptide
LKEFTVSGNILTIRLTAAD

SEQ ID NO: 37 - protein, 18 aa, Artificial Sequence
  synthetic peptide
LKEFTVSGNILTIRLTAA

SEQ ID NO: 38 - protein, 17 aa, Artificial Sequence
  synthetic peptide
LKEFTVSGNILTIRLTA

SEQ ID NO: 39 - protein, 16 aa, Artificial Sequence
  synthetic peptide
LKEFTVSGNILTIRLT

SEQ ID NO: 40 - protein, 15 aa, Artificial Sequence
  synthetic peptide
LKEFTVSGNILTIRL

SEQ ID NO: 41 - protein, 14 aa, Artificial Sequence
  synthetic peptide
LKEFTVSGNILTIR

SEQ ID NO: 42 - protein, 20 aa, Artificial Sequence
  synthetic peptide
KEFTVSGNILTIRLTAADHR

SEQ ID NO: 43 - protein, 19 aa, Artificial Sequence
  synthetic peptide
KEFTVSGNILTIRLTAADH

SEQ ID NO: 44 - protein, 18 aa, Artificial Sequence
  synthetic peptide
KEFTVSGNILTIRLTAAD

SEQ ID NO: 45 - protein, 17 aa, Artificial Sequence
  synthetic peptide
KEFTVSGNILTIRLTAA

SEQ ID NO: 46 - protein, 16 aa, Artificial Sequence
  synthetic peptide
KEFTVSGNILTIRLTA

SEQ ID NO: 47 - protein, 15 aa, Artificial Sequence
  synthetic peptide
KEFTVSGNILTIRLT

SEQ ID NO: 48 - protein, 14 aa, Artificial Sequence
  synthetic peptide
KEFTVSGNILTIRL

SEQ ID NO: 49 - protein, 13 aa, Artificial Sequence
  synthetic peptide
KEFTVSGNILTIR

SEQ ID NO: 50 - protein, 19 aa, Artificial Sequence
  synthetic peptide
EFTVSGNILTIRLTAADHR

SEQ ID NO: 51 - protein, 18 aa, Artificial Sequence
  synthetic peptide
EFTVSGNILTIRLTAADH

SEQ ID NO: 52 - protein, 17 aa, Artificial Sequence
  synthetic peptide
EFTVSGNILTIRLTAAD

SEQ ID NO: 53 - protein, 16 aa, Artificial Sequence
  synthetic peptide
EFTVSGNILTIRLTAA

SEQ ID NO: 54 - protein, 15 aa, Artificial Sequence
  synthetic peptide
EFTVSGNILTIRLTA

SEQ ID NO: 55 - protein, 14 aa, Artificial Sequence
  synthetic peptide
EFTVSGNILTIRLT

SEQ ID NO: 56 - protein, 13 aa, Artificial Sequence
  synthetic peptide
EFTVSGNILTIRL

SEQ ID NO: 57 - protein, 12 aa, Artificial Sequence
  synthetic peptide
EFTVSGNILTIR

SEQ ID NO: 58 - protein, 11 aa, Artificial Sequence
  synthetic peptide
TVSGNILTIRL

SEQ ID NO: 59 - protein, 9 aa, Artificial Sequence
  synthetic peptide
TVSGNILTI

SEQ ID NO: 60 - protein, 11 aa, Artificial Sequence
  synthetic peptide
EFTVSGNILTI

SEQ ID NO: 61 - protein, 18 aa, Artificial Sequence
  synthetic peptide
ICSANNRARGSYNEQFFG

SEQ ID NO: 62 - protein, 16 aa, Artificial Sequence
  synthetic peptide
ICSAFRRTDGDTQYFG

SEQ ID NO: 63 - protein, 18 aa, Artificial Sequence
  synthetic peptide
ICSARDMGTAEVYGYTFG

SEQ ID NO: 64 - protein, 16 aa, Artificial Sequence
  synthetic peptide
ICSVASRREGEEQYFG

SEQ ID NO: 65 - protein, 18 aa, Artificial Sequence
  synthetic peptide
ICSARDERGGRYNEQFFG

SEQ ID NO: 66 - protein, 16 aa, Artificial Sequence
  synthetic peptide
ICSAYPGVTNEKLFFG

SEQ ID NO: 67 - protein, 19 aa, Artificial Sequence
  synthetic peptide
ICSASSPGTSGRAGELFFG

SEQ ID NO: 68 - protein, 18 aa, Artificial Sequence
  synthetic peptide
ICSARGGLPSSYNEQFFG

SEQ ID NO: 69 - protein, 18 aa, Artificial Sequence
  synthetic peptide
ICSARDPSKSSYNEQFFG

SEQ ID NO: 70 - protein, 18 aa, Artificial Sequence
  synthetic peptide
ICSARGPGQGIGDTQYFG

SEQ ID NO: 71 - protein, 16 aa, Artificial Sequence
  synthetic peptide
ICSARGAGNTGELFFG

SEQ ID NO: 72 - protein, 16 aa, Artificial Sequence
  synthetic peptide
ICSLIRADTNTEAFFG

SEQ ID NO: 73 - protein, 18 aa, Artificial Sequence
  synthetic peptide
ICSARGASGANYNEQFFG

SEQ ID NO: 74 - DNA, 29 bp, Artificial Sequence
  synthetic polynucleotide
ctttagatctcgaccacgtttcttggagc

SEQ ID NO: 75 - DNA, 30 bp, Artificial Sequence
  synthetic polynucleotide
ctttgaattccttgctctgtgcagattcag

SEQ ID NO: 76 - DNA, 31 bp, Artificial Sequence
  synthetic polynucleotide
ctttagatctatcaaagaagaacatgtgatc

SEQ ID NO: 77 - DNA, 29 bp, Artificial Sequence
  synthetic polynucleotide
cttcgaattcgttctctgtagtctctggg

SEQ ID NO: 78 - DNA, 717 bp, Artificial Sequence
  synthetic polynucleotide
agatctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacgaacagccctgtggaactgagagagcccaacgtcctcatctgtttcatcgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgaattcggtggtggatcaggaggttcaactacagctccatcagctcagctcgaaaaagagctccaggccctggagaaggaaaatgcacagctggaatgggagttgcaagcactggaaaaggaactggctcagtaa

SEQ ID NO: 79 - protein, 238 aa, Artificial Sequence
  synthetic peptide
RSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENEFGGGSGGSTTAPSAQLEKELQALEKENAQLEWELQALEKELAQ

SEQ ID NO: 80 - DNA, 786 bp, Artificial Sequence
  synthetic polynucleotide
agatctcgaccacgtttcttggagctgcttaagtctgagtgtcatttcttcaatgggacggagcgggtgcggttcctggagagacacttccataaccaggaggagtacgcgcgcttcgacagcgacgtgggggagtaccgggcggtgagggagctggggcggcctgatgccgagtactggaacagccagaaggacctcctggagcagaagcggggccaggtggacaattactgcagacacaactacggggttggtgagagcttcacagtgcagcggcgagtccatcctcaggtgactgtgtatcctgcaaagacccagcccctgcagcaccacaacctcctggtctgctctgtgagtggtttctatccaggcagcattgaagtcaggtggttccggaacggccaggaagagaaggctggggtggtgtccacgggcctgatccagaatggagactggaccttccagaccctggtgatgctagaaacagttcctcggagtggagaggtttacacctgccaagtggagcacccaagcgtaacgagccctctcacagtggaatggagtgcacggtctgaatctgcacagagcaaggaattcggtggtggatcaggaggttcaactacagctccatcagctcagttgaaaaagaaattgcaagcactgaagaaaaagaacgctcagctgaagtggaaacttcaagccctcaagaagaaactcgcccagggaggcagtggtggcggtctgaacgacatcttcgaggctcagaaaatcgaatggcacgaatga

SEQ ID NO: 81 - protein, 261 aa, Artificial Sequence
  synthetic peptide
RSRPRFLELLKSECHFFNGTERVRFLERHFHNQEEYARFDSDVGEYRAVRELGRPDAEYWNSQKDLLEQKRGQVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSPLTVEWSARSESAQSKEFGGGSGGSTTAPSAQLKKKLQALKKKNAQLKWKLQALKKKLAQGGSGGGLNDIFEAQKIEWHE

SEQ ID NO: 82 - protein, 9 aa, Artificial Sequence
  synthetic peptide
TAADHRQLQ

SEQ ID NO: 83 - DNA, 29 bp, Artificial Sequence
  synthetic polynucleotide
ctttagatctcgaccacgtttcttggagc

SEQ ID NO: 84 - DNA, 30 bp, Artificial Sequence; identical to SEQ ID NO: 75
  synthetic polynucleotide

SEQ ID NO: 85 - protein, 6 aa, Artificial Sequence
  synthetic peptide
DTYRYI

SEQ ID NO: 86 - protein, 6 aa, Artificial Sequence
  synthetic peptide
TDFYLK

SEQ ID NO: 87 - protein, 11 aa, Artificial Sequence
  synthetic peptide
MASMTGGQQMG

SEQ ID NO: 88 - protein, 14 aa, Artificial Sequence
  synthetic peptide
GKPIPNPLLGLDST

SEQ ID NO: 89 - protein, 6 aa, Artificial Sequence
  synthetic peptide
QYPALT

SEQ ID NO: 90 - protein, 10 aa, Artificial Sequence
  synthetic peptide
SSTSSDFRDR

SEQ ID NO: 91 - protein, 7 aa, Artificial Sequence
  synthetic peptide
DYKDDDK

SEQ ID NO: 92 - protein, 17 aa, Artificial Sequence
  synthetic peptide
PGVLLKEFTVSGNILTI

SEQ ID NO: 93 - protein, 17 aa, Artificial Sequence
  synthetic peptide
PGVLLKEFTVSGNILTI

SEQ ID NO: 94 - protein, 20 aa, Artificial Sequence
  synthetic peptide
MQAEGRGTGGSTGDADGPGG

SEQ ID NO: 95 - protein, 19 aa, Artificial Sequence
  synthetic peptide
KDHLIHNVHKEFHAHAHNK

SEQ ID NO: 96 - protein, 11 aa, Artificial Sequence
  synthetic peptide
KPPTPPPEPET

SEQ ID NO: 97 - protein, 11 aa, Artificial Sequence
  synthetic peptide
CEQKLISEEDL

SEQ ID NO: 98 - protein, 5 aa, Artificial Sequence
  synthetic peptide
TEFCA

SEQ ID NO: 99 - protein, 5 aa, Artificial Sequence
  synthetic peptide
RRRRR

SEQ ID NO: 100 - skipped

SEQ ID NO: 101 - protein, 4 aa, Artificial Sequence
  synthetic peptide
CCCC

SEQ ID NO: 102 - protein, 3 aa, Artificial Sequence
  synthetic peptide
HHH

SEQ ID NO: 103 - protein, 4 aa, Artificial Sequence
  synthetic peptide
HHHH

SEQ ID NO: 104 - protein, 5 aa, Artificial Sequence
  synthetic peptide
HHHHH

SEQ ID NO: 105 - protein, 6 aa, Artificial Sequence
  synthetic peptide
HHHHHH

SEQ ID NO: 106 - protein, 7 aa, Artificial Sequence
  synthetic peptide
HHHHHHH

SEQ ID NO: 107 - protein, 8 aa, Artificial Sequence
  synthetic peptide
HHHHHHHH

SEQ ID NO: 108 - protein, 9 aa, Artificial Sequence
  synthetic peptide
HHHHHHHHH

SEQ ID NO: 109 - protein, 10 aa, Artificial Sequence
  synthetic peptide
HHHHHHHHHH

SEQ ID NO: 110 - protein, 11 aa, Artificial Sequence
  synthetic peptide
HHHHHHHHHHH

SEQ ID NO: 111 - protein, 12 aa, Artificial Sequence
  synthetic peptide
HHHHHHHHHHHH

SEQ ID NO: 112 - protein, 11 aa, Artificial Sequence
  synthetic peptide
FFFFFFFFFFF

SEQ ID NO: 113 - protein, 9 aa, Artificial Sequence
  synthetic peptide
NANNPDWDF

SEQ ID NO: 114 - protein, 15 aa, Artificial Sequence
  synthetic peptide
KETAAAKFERQHMDS

SEQ ID NO: 115 - protein, 8 aa, Artificial Sequence
  synthetic peptide
WSHPQFEK

SEQ ID NO: 116 - protein, 9 aa, Artificial Sequence
  synthetic peptide
AWAHPQPGG

SEQ ID NO: 117 - protein, 6 aa, Artificial Sequence
  synthetic peptide
HTTPHH

SEQ ID NO: 118 - protein, 11 aa, Artificial Sequence
  synthetic peptide
YTDIEMNRLGK

SEQ ID NO: 119 - protein, 4 aa, Artificial Sequence
  synthetic peptide
SGSG

SEQ ID NO: 120 - protein, 9 aa, Artificial Sequence
  synthetic peptide
YIKGNRKPI

SEQ ID NO: 121 - protein, 15 aa, Artificial Sequence
  synthetic peptide
DEPTLLYVLFEVFDV

SEQ ID NO: 122 - protein, 15 aa, Artificial Sequence
  synthetic peptide
PVSKMRMATPLLMQA

SEQ ID NO: 123 - protein, 15 aa, Artificial Sequence
  synthetic peptide
RKVAELVHFLLLKYR

SEQ ID NO: 124 - protein, 16 aa, Artificial Sequence
  synthetic peptide
KKLLTQHFVQENYLEY

SEQ ID NO: 125 - protein, 14 aa, Artificial Sequence
  synthetic peptide
SLLMWITQCFLPVF

SEQ ID NO: 126 - protein, 24 aa, Artificial Sequence
  synthetic peptide
SLLMWITQCFLPVFLAQPPSGQRR

SEQ ID NO: 127 - protein, 14 aa, Artificial Sequence
  synthetic peptide
FNNFTVSFWLRVPK

SEQ ID NO: 128 - protein, 20 aa, Artificial Sequence
  synthetic peptide
QILDGENCTLIDALLGDPQD

SEQ ID NO: 129 - protein, 15 aa, Artificial Sequence
  synthetic peptide
GRAMLGTHTMEVTVY

SEQ ID NO: 130 - protein, 12 aa, Artificial Sequence
  synthetic peptide
EEAAGIGILTVI

SEQ ID NO: 131 - protein, 10 aa, Artificial Sequence
  synthetic peptide
EAAGIGILTV

SEQ ID NO: 132 - protein, 15 aa, Artificial Sequence
  synthetic peptide
PVSKMRMATPLLMQA

SEQ ID NO: 133 - protein, 13 aa, Artificial Sequence
  synthetic peptide
PKYVKQNTLKLAT

SEQ ID NO: 134 - protein, 16 aa, Artificial Sequence
  synthetic peptide
ACYEFLWGPRALVETS

SEQ ID NO: 135 - protein, 12 aa, Artificial Sequence
  synthetic peptide
LLEFYLAMPFAT

SEQ ID NO: 136 - protein, 15 aa, Artificial Sequence
  synthetic peptide
LKEFTVSGNILTIRL

SEQ ID NO: 137 - protein, 15 aa, Artificial Sequence
  synthetic peptide
PVSKMRMATPLLMQA

SEQ ID NO: 138 - protein, 16 aa, Artificial Sequence
  synthetic peptide
WNRQLYPEWTEAQRLD

SEQ ID NO: 139 - protein, 12 aa, Artificial Sequence
  synthetic peptide
GFVFTLTVPSER

SEQ ID NO: 140 - protein, 24 aa, Artificial Sequence
  synthetic peptide
FWRGENGRKTRIAYERMCNILKGK

SEQ ID NO: 141 - protein, 25 aa, Artificial Sequence
  synthetic peptide
PGVLLKEFTVSGNILTIRLTAADHR

SEQ ID NO: 142 - protein, 17 aa, Artificial Sequence
  synthetic peptide
ISQAVHAAHAEINEAGR

SEQ ID NO: 143 - protein, 15 aa, Artificial Sequence
  synthetic peptide
KFGWSGPDCNRKKPA

SEQ ID NO: 144 - protein, 20 aa, Artificial Sequence
  synthetic peptide
GLNGPDIYKGVYQFKSVEFD

SEQ ID NO: 145 - protein, 15 aa, Artificial Sequence
  synthetic peptide
ADIYTFPLENAPIGH